SEQUENCE LISTING (33 sequences)

SEQ ID NO: 1 - protein, 972 aa, synthetic construct
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 2 - DNA, 3978 bp, synthetic construct
  source 1..3978: mol_type = other DNA
agacagagtgtccaaaagcgtgagagcacgaagtgaggagaaggtggagaagagagaagaggaagaggaagaggaagagaggaagcggagggaactgcggccaggctaaaaggggaagaagaggatcagcccaaggaggaggaagaggaaaacaagacaaacagccagtgcagaggagaggaacgtgtgtccagtgtcccgatccctgcggagctagtagctgagagctctgtgccctgggcaccttgcagccctgcacctgcctgccacttccccaccgaggccatgggcccaggagttctgctgctcctgctggtggccacagcttggcatggtcagggaatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctgaggagttgacgacagggagtaccactctcccctcccacaaacttcaactcctccatggatggggcgacacggggagaacatacaaactctgccttcggtcatttcactcaacagctcggcccagctctgaaacttgggaaggtgagggattcaggggaggtcagaggatcccacttcctgagcatgggccatcactgccagtcaggggctgggggctgagccctcacccccccctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctcacaggacctcttagtctctgccctatacgccgccttcactccacagcctcacccctcccacccccatactggtactgctgtaatgagccaagtggcagctaaaagttgggggtgttctgcccagtcccgtcattctgggctagaaggcaggggaccttggcatgtggctggccacaccaagcaggaagcacaaactcccccaagctgactcatcctaactaacagtcacgccgtgggatgtctctgtccacattaaactaacagcattaatgca

SEQ ID NO: 3 - protein, 446 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 4 - protein, 220 aa, synthetic construct
DIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, synthetic construct
SYGIS

SEQ ID NO: 6 - protein, 8 aa, synthetic construct
GYTFTSYG

SEQ ID NO: 7 - protein, 7 aa, synthetic construct
GYTFTSY

SEQ ID NO: 8 - protein, 17 aa, synthetic construct
WISAYNGNTNYAQKLQG

SEQ ID NO: 9 - protein, 8 aa, synthetic construct
ISAYNGNT

SEQ ID NO: 10 - protein, 6 aa, synthetic construct
SAYNGN

SEQ ID NO: 11 - protein, 11 aa, synthetic construct
ESWFGEVFFDY

SEQ ID NO: 12 - protein, 13 aa, synthetic construct
ARESWFGEVFFDY

SEQ ID NO: 13 - protein, 17 aa, synthetic construct
KSSQSVLDSSDNKNYLA

SEQ ID NO: 14 - protein, 12 aa, synthetic construct
QSVLDSSDNKNY

SEQ ID NO: 15 - protein, 7 aa, synthetic construct
WASNRES

SEQ ID NO: 16 - skipped

SEQ ID NO: 17 - protein, 9 aa, synthetic construct
QQYYSDPFT

SEQ ID NO: 18 - protein, 554 aa, synthetic construct
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 19 - DNA, 3994 bp, synthetic construct
  source 1..3994: mol_type = other DNA
gaggctcggcccggggaaagtgaaagtttgcctgggtcctctcggcgccagagccgctctccgcatcccaggacagcggtgcggccctcggccggggcgcccactccgcagcagccagcgagcgagcgagcgagcgagggcggccgacgcgcccggccgggacccagctgcccgtatgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactggtaccgccttgcccagggtgggccccgtgaggcccactggccaggactggaatcacaccccccagaagacagaccatccatctgccctgctcagagaccccccggagccaggctctcccaggatctcatcactgcgcccccagggcctcagcaacccctccaccctctctgctcagccacagctttccagaagccactcctcgggcagcgtgctgccccttggggagctggagggcaggaggagcaccagggatcggaggagccccgcagagccagaaggaggaccagcaagtgaaggggcagccaggcccctgccccgttttaactccgttcctttgactgacacaggccatgagaggcagtccgagggatccttcagcccgcagctccaggagtctgtcttccacctgctggtgcccagtgtcatcctggtcttgctggccgtcggaggcctcttgttctacaggtggaggcggcggagccatcaagagcctcagagagcggattctcccttggagcaaccagagggcagccccctgactcaggatgacagacaggtggaactgccagtgtagagggaattctaagctggacgcacagaacagtctctccgtgggaggagacattatggggcgtccaccaccacccctccctggccatcctcctggaatgtggtctgccctccaccagagctcctgcctgccaggactggaccagagcagccaggctggggcccctctgtctcaacccgcagacccttgactgaatgagagaggccagaggatgctccccatgctgccactatttattgtgagccctggaggctcccatgtgcttgaggaaggctggtgagcccggctcaggaccctcttccctcaggggctgcaccctcctctcactcccttccatgccggaacccaggccagggacccaccggcctgtggtttgtgggaaagcagggtggacgctgaggagtgaaagaaccctgcacccagagggcctgcctggtgccaaggtatcccagcctggacaggcatggacctgtctccagagagaggagcctgaagttcgtggggcgggacagcgtcggcctgatttcccgtaaaggtgtgcagcctgagagacgggaagaggaggcctctggacctgctggtctgcactgacagcctgaagggtctacaccctcggctcacctaagtgccctgtgctggttgccaggcgcagaggggaggccagccctgccctcaggacctgcctgacctgccagtgatgccaagagggggatcaagcactggcctctgcccctcctccttccagcacctgccagagcttctccaggaggccaagcagaggctcccctcatgaaggaagccattgcactgtgaacactgtacctgcctgctgaacagcctgcccccgtccatccatgagccagcatccgtccgtcctccactctccagcctctccccagcctcctgcactgagctggcctcaccagtcgactgagggagcccctcagccctgaccttctcctgacctggcctttgactccccggagtggagtggggtgggagaacctcctgggccgccagccagagccggtctttaggctgtgttgttcgcccaggtttctgcatcttgcactttgacattcccaagagggaagggactagtgggagagagcaagggaggggagggcacagacagagaggctacagggcgagctctgactgaagatgggcctttgaaatataggtatgcacctgaggttgggggagggtctgcactcccaaaccccagcgcagtgtcctttccctgctgccgacaggaacctggggctgaacaggttatccctgtcaggagccctggactgggctgcatctcagccccacctgcatggtatccagctcccatccacttctcacccttctttcctcctgaccttggtcagcagtgatgacctccaactctcacccaccccctctaccatcacctctaaccaggcaagccagggtgggagagcaatcaggagagccaggcctcagcttccaatgcctggagggcctccactttgtggccagcctgtggtggtggctctgaggcctaggcaacgagcgacagggctgccagttgcccctgggttcctttgtgctgctgtgtgcctcctctcctgccgccctttgtcctccgctaagagaccctgccctacctggccgctgggccccgtgactttcccttcctgcccaggaaagtgagggtcggctggccccaccttccctgtcctgatgccgacagcttagggaagggcagtgaacttgcatatggggcttagccttctagtcacagcctctatatttgatgctagaaaacacatatttttaaatggaagaaaaataaaaaggcattcccccttcatccccctaccttaaacatataatattttaaaggtcaaaaaagcaatccaacccactgcagaagctctttttgagcacttggtggcatcagagcaggaggagccccagagccacctctggtgtccccccaggctacctgctcaggaaccccttctgttctctgagaagtcaagagaggacattggctcacgcactgtgagattttgtttttatacttggaagtggtgaattattttatataaagtcatttaaatatctatttaaaagataggaagctgcttatatatttaataataaaagaagtgcacaagctgc

SEQ ID NO: 20 - protein, 446 aa, synthetic construct
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 213 aa, synthetic construct
DIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 449 aa, synthetic construct
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 218 aa, synthetic construct
EIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 450 aa, synthetic construct
QDQLVESGGGVVQPGRSLRLSCAASGFTFSSYGMHWVRQAPGEGLEWVAVIWYDGSNKYYADSVKGRFTISRDNSKNTLYLQMNSLRAEDTAVYYCARGDYEVDYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 214 aa, synthetic construct
AIQLTQSPSSLSASVGDRVTITCRASQGISNALAWYQQKPGKAPKLLIYDASSLESGVPSRFSGSGSGTDFTLTISSLQPEDFATYYCQQFNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 453 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 214 aa, synthetic construct
DIQMTQSPSSLSASVGDRVTITCLASEDIYDNLAWYQQKPGKAPKLLIYYASSLQDGVPSRFSGSGSGTDYTLTISSLQPEDFATYYCLQDSEYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 448 aa, synthetic construct
QVQLQESGPGLVKPSQTLSLTCTVSDYSITSDYAWNWIRQFPGKGLEWMGYISYSGSTSYNPSLKSRITISRDTSKNQFSLQLNSVTAADTAVYYCASFDYAHAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 214 aa, synthetic construct
DIVLTQSPAFLSVTPGEKVTFTCQASQSIGTSIHWYQQKTDQAPKLLIKYASESISGIPSRFSGSGSGTDFTLTISSVEAEDAADYYCQQINSWPTTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 447 aa, synthetic construct
DVQLQESGPGLVKPSQSLSLTCTVTDYSITSDYAWNWIRQFPGNKLEWMGYISYSGSTSYNPSLKSRISITRDTSKNQFFLQLNSVTTEDTATYYCASFDYAHAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 31 - protein, 214 aa, synthetic construct
DILLTQSPAILSVSPGERVSFSCRASQSIGTSIHWYQQRTNGSPRLLIKYASESISGIPSRFSGSGSGTDFTLSINSVESEDIADYYCQQINSWPTTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 447 aa, synthetic construct
EVQLVESGGGLVQPGGSLRLSCAASGFTFSSFSMTWVRQAPGKGLEWVSYISSRSSTISYADSVKGRFTISRDNAKNSLYLQMNSLRDEDTAVYYCARDPLLAGATFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 215 aa, synthetic construct
EFVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0313640 A1
(43) Pub. Date: Oct. 9, 2025

(54) INHIBITION OF CSF-1 or CSF-1R FOR THE TREATMENT OF MINIMAL RESIDUAL DISEASE

(71) Applicant: BOARD OF REGENTS, THE UNIVERSITY OF TEXAS SYSTEM, Austin, TX (US)

(72) Inventors: David G. MENTER, Houston, TX (US); Edmund KOPETZ, Houston, TX (US); Natalie Wall FOWLKES, Houston, TX (US); Dipen Maheshbhai MARU, Houston, TX (US); Alaa Mohamed Tamin MOHAMED, Houston, TX (US); Linghua WANG, Houston, TX (US); Van Karlyle MORRIS, Houston, TX (US)

(73) Assignee: BOARD OF REGENTS, THE UNIVERSITY OF TEXAS SYSTEM, Austin, TX (US)

(21) Appl. No.: 19/171,053

(22) Filed: Apr. 4, 2025

Related U.S. Application Data
(60) Provisional application No. 63/575,136, filed on Apr. 5, 2024.

(51) Int. Cl. C07K 16/28; A61K 39/00; A61P 35/04; G01N 33/53

(52) U.S. Cl. CPC C07K 16/2866; A61P 35/04; G01N 33/5308; A61K 2039/505; C07K 2317/24; C07K 2317/76; G01N 2800/52; G01N 2800/54

(57) ABSTRACT

Embodiments of the disclosure include methods and compositions for treating minimal residual disease in an individual. In specific embodiments, the disclosure concerns methods of treating an individual with minimal residual disease with one or more inhibitors of CSF-1R and/or one or more inhibitors of CSF-1. In specific embodiments, the individual is positive for the presence of mutated circulating tumor DNA (ctDNA), and/or the individual has colorectal cancer.

### BACKGROUND

This application claims priority to U.S. Provisional Patent Application Ser. No. 63/575, 136, filed Apr. 5, 2024, which is incorporated by reference herein in its entirety.

The application contains a Sequence Listing in compliance with ST. 26 format and is hereby incorporated by reference in its entirety. Said Sequence Listing, created on Mar. 12, 2025 is named MDACP1412US_Sl.xml and is 43,455 bytes in size.

### TECHNICAL FIELD

This disclosure relates at least to the fields of immunology, cell biology, molecular biology, and therapeutics.

### BACKGROUND

The high mortality rate of cancer is primarily due to metastatic disease. The most common initial site of distant metastasis is the liver. The presence of microscopic minimal residual disease (MRD) detected in patients by circulating tumor DNA (ctDNA) may remain radiographically undetectable and persist following therapeutic intervention, eventually resulting in recurrence. In patients who have already completed adjuvant therapy, there is currently no efficacious treatment regimen that targets and eliminates MRD before progression to radiologically evident disease. Preclinical models are urgently needed to elucidate the mechanisms whereby MRD escapes anti-tumor immune surveillance. The present disclosure involves development and characterization of a preclinical model that accurately recapitulates immune response to MRD in the liver. Uncovering the immunobiology of MRD accelerates the discovery of novel, druggable targets and changes clinical management of ctDNA positive patients to improved outcomes.

### SUMMARY

Embodiments of the disclosure include methods and compositions for treating cancer, including cancer that is present as minimal residual disease (MRD), in at least some cases. An individual identified as having MRD may be subject to methods of the disclosure, although in some cases an individual unknown to have MRD, including suspected of having MRD, may be subject to methods of the disclosure. The individual may not be known to have MRD because the individual has not been tested or because the individual does not have detectable MRD. In some embodiments, the individual is subject to methods of the disclosure as a part of routine medical care. In some embodiments, the individual is subject to methods of the disclosure as a part of routine medical care following one or more cancer therapies. In some embodiments, the individual is subject to methods of the disclosure that are not as a part of routine medical care. The individual may be positive for one or more markers of disease, including MRD, such as mutated circulating tumor DNA (ctDNA).

In some embodiments, there is microscopic MRD that may or may not be detected in individuals having ctDNA. In some embodiments, an individual has MRD that is not detectable by routine assays in the art, including at least radiographically undetectable. In some embodiments, an individual has MRD that persists following therapeutic intervention and may result in recurrence. In some embodiments, the individual has completed or is receiving adjuvant therapy of any kind. In some embodiments, the MRD has not progressed to radiologically-evident (or evident by one or more assays) disease. In some embodiments, MRD in the individual has escaped anti-tumor immune surveillance.

Embodiments of the disclosure provide methods for improving, such as lengthening and/or hastening, a window of treatment for an individual in need thereof. The individual may have MRD or may be suspected of having MRD and be in need of further treatment as a result, and in some embodiments the individual is provided a therapeutically effective amount of one or more inhibitors of colony stimulating factor 1 receptor (CSF1 or CSF-1) and/or one or more inhibitors of colony stimulating factor 1 receptor (CSF1R or CSF-1R). An individual may be provided a therapeutically effective amount of one or more inhibitors of CSF-1 and/or one or more inhibitors of CSF-1R as part of routine care, such as routine care following one or more cancer therapies. In such cases, the individual may have any type of cancer, whereas in other cases the individual may have colorectal cancer and/or liver cancer. In certain aspects, the methods of disclosure allow for an individual to be treated for MRD earlier than if the individual had not been determined to have MRD, and in specific cases only, the individual has colorectal cancer and/or liver cancer, has circulating tumor DNA (ctDNA), has mutated ctDNA, or a combination thereof.

In some embodiments, an individual with cancer is treated with curative intent and, following the curative intent, the individual is administered a therapeutically effective amount of one or more inhibitors of CSF-1 and/or one or more inhibitors of CSF-1R. The individual may or may not be known to have MRD. The individual may or may not be known to be positive for ctDNA. The individual may have metastatic cancer, may be at risk for metastatic cancer, or may be suspected of having metastatic cancer. In some cases the individual has any cancer, whereas in other cases the individual has colorectal cancer and/or liver. In particular embodiments, the individual is provided a therapeutically effective amount of one or more inhibitors of CSF-1R whether or not the individual is known to be positive for MRD, positive for ctDNA, or both. The individual may be subject to surveillance of MRD, the presence of ctDNA, or both. The individual may be subject to any method of the disclosure as part of maintenance therapy. Cancer in the individual may be radiographically undetectable and/or undetectable by any other assay. In some embodiments, an individual subject to a method of the disclosure does not have detectable cancer tissue, including following therapy for the cancer. The cancer of the individual may be high microsatellite instability (MSI-H), low microsatellite instability (MSI-L) or microsatellite stability (MSS).

In embodiments wherein the CSF-1 inhibitor and/or CSF-1R inhibitor is an antibody, the antibody may be further defined as human, chimeric, humanized, or murine, as example. The antibody or antigen binding fragment may be further defined as antagonistic. The antibody may be one that includes or excludes a human antibody, humanized antibody, recombinant antibody, chimeric antibody, an antibody derivative, a veneered antibody, a diabody, a monoclonal antibody, a single domain antibody, or a single chain antibody. The antigen binding fragment may include or exclude a single chain variable fragment (scFv), F(ab′)2, Fab′, Fab, Fv, or rIgG. The antigen binding fragment may comprise a scFv. The VH may be amino proximal to the VL. The VL may be amino proximal to the VH. A first region is carboxy-proximal to a second region when the first region is attached to the carboxy terminus of the second region. There may be further intervening amino acid residues between the first and second regions. Thus, the regions need not be immediately adjacent, unless specifically specified as not having intervening amino acid residues. The term “amino-proximal” is similarly defined in that a first region is amino-proximal to a second region when the first region is attached to the amino terminus of the second region. Similarly, there may be further intervening amino acid residues between the first and second regions unless stated otherwise. The antigen binding fragment may comprise a VH and a VL and a linker between the VH and VL. The linker may comprise or exclude a glycine serine linker.

The methods may further comprise administering an additional therapy to the individual, including other than the CSF-1 inhibitor therapy and/or the CSF-1R inhibitor therapy. The additional therapy may comprise or exclude an immunotherapy, anti-angiogenic therapy, chemotherapy, surgery, radiotherapy, neoantigen therapy, or vaccination. The additional therapy may comprise or exclude an immunotherapy and such as wherein the immunotherapy comprises immune checkpoint inhibitor therapy, adoptive cell therapy, or bispecific T cell engagers, as examples. The cells may be autologous or non-autologous. The subject or patient may be a human subject. The subject or patient may be further defined as a mammal, a non-human primate, a pig, horse, mouse, rat, rabbit, or dog.

The therapeutic agents of the disclosure may be used for in vivo, in vitro, or ex vivo administration. The route of administration of the therapies may be, for example, intracutaneous, subcutaneous, intravenous, local, topical, and intraperitoneal administrations.

In some embodiments, the disclosed methods are directed to methods for treating cancer. The cancer may be a hematological cancer, solid tumor, metastatic cancer, or non-metastatic cancer. In certain embodiments, the cancer may be recurrent, metastatic, relapsed, or of a Stage I, II, III, or IV.

Aspects of the disclosure include methods of treating cancer in an individual, comprising administering to the individual a therapeutically effective amount of one or more inhibitors of CSF-1 and/or one or more inhibitors of CSF-1R. In some embodiments, one or more inhibitors of CSF-1 are utilized for therapy prior to using one or more inhibitors of CSF-1R for therapy, and vice versa. The individual may have MRD and/or circulating tumor DNA (ctDNA). The individual may have colorectal, skin, breast, lung, liver, stomach, brain, thyroid, blood, gall bladder, ovarian, cervical, testicular, prostate, kidney, pancreatic, small bowel, gastric, mesenteric, GI lymph node, colon, or bladder cancer, or any cancer that engages the portal vein and/or drains into the liver. In specific aspects, the individual has colorectal cancer and/or liver cancer. The individual may have both colorectal cancer and ctDNA, although in some cases wherein the individual has ctDNA, it may not be detectable.

In specific aspects, any one or more of the inhibitors is a protein, nucleic acid, small molecule, combination thereof or mixture thereof. The one or more inhibitors may be a kinase inhibitor. The inhibitor may be a protein that is an antibody, a ligand of CSF-1R, or a combination thereof. The antibody may be AMB001. The antibody may have a sequence of any one or more of SEQ ID NO:5-17 and/or SEQ ID NO:20-33. In certain aspects, the one or more inhibitors is a small molecule, including one or more of the small molecules in FIG. 1. In some embodiments, the one or more inhibitors is a ligand of CSF-1R, such as CSF-1, IL-34, or a mixture thereof.

Any method encompassed herein may further comprise the step of identifying that the individual has MRD. In specific embodiments, a sample from an individual for testing for

MRD comprises blood, bone marrow aspirate, or both. The method may further comprise identifying that the individual has ctDNA. In some embodiments, in addition to assaying whether the individual has ctDNA, or alternatively, one may assay for identification of MRD by assaying for the following: methylated DNA, circulating RNA, protein(s), small molecule(s), and/or metabolic marker(s), and one or more of these may or may not be from cancer cells.

Aspects of the disclosure include methods of determining a risk for recurrent cancer in an individual, comprising providing an effective amount of one or more inhibitors of CSF-1 or one or more inhibitors of CSF-1R to an individual that has ctDNA or that has undetectable ctDNA.

Aspects of the disclosure include methods of determining a risk for recurrent cancer in an individual, comprising assaying for whether or not an individual has ctDNA; and administering an effective amount of one or more inhibitors of CSF-1 or one or more inhibitors of CSF-1R to an individual that has ctDNA, although in some embodiments the individual may be determined to have a risk for recurrent cancer by another one or more means and is administered one or more inhibitors of CSF-1 or one or more inhibitors of CSF-1R. In specific embodiments, prior to determining whether the individual has ctDNA, the method further comprises administering to the individual one or more cancer therapies.

Certain embodiments of the present disclosure are characterized through the following enumerated aspects.

Aspect 1. A method of treating minimal residual disease in an individual, comprising administering to the individual a therapeutically effective amount of one or more inhibitors of colony stimulating factor 1 (CSF-1) and/or one or more inhibitors of colony stimulating factor 1 receptor (CSF-1R).

Aspect 2. The method of aspect 1, wherein the individual has circulating tumor DNA (ctDNA).

Aspect 3. The method of aspect 1 or 2, wherein the individual has undetected or undetectable cancer cells.

Aspect 4. The method of any one of aspects 1-3, wherein the individual has radiographically undetectable cancer cells.

Aspect 5. The method of any one of aspects 1-4, wherein the individual has no detected ctDNA but is at risk of developing MRD or is suspected of having MRD.

Aspect 6. The method of any one of aspects 1-5, wherein the cancer has high microsatellite instability (MSI-H), low microsatellite instability (MSI-L) or microsatellite stability (MSS).

Aspect 7. The method of any one of aspects 1-6, wherein the individual does not have detectable cancer tissue.

Aspect 8. The method of any one of aspects 1-7, wherein the individual has received therapy for the cancer.

Aspect 9. The method of any one of aspects 1-8, wherein the administering is for maintenance therapy or is part of routine care.

Aspect 10. The method of any one of aspects 1-9, wherein one or more tumors in the individual is <0.5 mm.

Aspect 11. The method of any one of aspects 1-10, wherein the individual has colorectal, skin, breast, lung, liver, stomach, brain, thyroid, blood, gall bladder, ovarian, cervical, testicular, prostate, kidney, pancreatic, small bowel, gastric, mesenteric, GI lymph node, colon, or bladder cancer, or any cancer that engages the portal vein and/or drains into the liver.

Aspect 12. The method of any one of aspects 1-11, wherein the individual has colorectal cancer that has metastasized to the liver.

Aspect 13. The method of any one of aspects 1-12, wherein the individual has colorectal cancer and has detectable ctDNA.

Aspect 14. The method of any one of aspects 1-13, wherein the one or more inhibitors is a protein, nucleic acid, small molecule, mixture thereof, or combination thereof.

Aspect 15. The method of any one of aspects 1-14, wherein the one or more inhibitors is a kinase inhibitor.

Aspect 16. The method of any one of aspects 1-15, wherein the inhibitor is a ligand of CSF-1R.

Aspect 17. The method of aspect 16, wherein the ligand of CSF-1R is CSF-1, IL-34, or a mixture thereof.

Aspect 18. The method of any one of aspects 14-16, wherein the protein is an antibody.

Aspect 19. The method of aspect 18, wherein the antibody is AMB001.

Aspect 20. The method of aspect 18 or 19, wherein the antibody has a sequence of any one or more of SEQ ID NO:5-17 or SEQ ID NO:20-33.

Aspect 21. The method of aspect 14, wherein the one or more inhibitors is a small molecule.

Aspect 22. The method of aspect 21, wherein the one or more inhibitors is one or more of the small molecules in FIG. 1.

Aspect 23. The method of any one of aspects 1-22, wherein the method further comprises the step of identifying that the individual has MRD.

Aspect 24. The method of aspect 23, wherein a sample from an individual for testing for MRD comprises blood, bone marrow aspirate, solid tumor tissue, or a combination thereof.

Aspect 25. The method of any one of aspects 1-24, wherein the method further comprises assaying whether the individual has ctDNA.

Aspect 26. The method of any one of aspects 1-25, wherein the inhibitor is an inhibitor of CSF-1.

Aspect 27. The method of any one of aspects 1-25, wherein the inhibitor is an inhibitor of CSF-R1.

Aspect 28. A method of determining a risk for recurrent cancer in an individual, comprising providing an effective amount of an inhibitor of CSF-1R to an individual that has ctDNA.

Aspect 29. A method of determining a risk for recurrent cancer in an individual, comprising:

determining whether an individual has ctDNA; and

administering an effective amount of an inhibitor of CSF-1R or CSF-1 to an individual that has ctDNA.

Aspect 30. The method of aspect 28, wherein prior to determining whether the individual has ctDNA, the method further comprises administering to the individual one or more cancer therapies.

Aspect 31. A method, comprising administering a therapeutically effective amount of an inhibitor of CSF-1R to an individual that has ctDNA.

Aspect 32. A method, comprising:

determining whether an individual has ctDNA; and

administering an effective amount of one or more inhibitors of CSF-1R and/or one or more inhibitors of CSF-1 to an individual that has ctDNA.

Aspect 33. A method of treating an individual after one or more cancer therapies, comprising administering after the cancer therapy to the individual a therapeutically effective amount of one or more inhibitors of CSF-1 and/or one or more inhibitors of CSF-1R if the individual has ctDNA.

Aspect 34. The method of aspect 33, further comprising assaying for ctDNA in a sample from the individual.

Aspect 35. The method of aspect 34, wherein no ctDNA is detected in the individual.

Aspect 36. The method of aspect 34, wherein ctDNA is detectable in the individual.

Aspect 37. The method of any one of aspects 33-36, wherein the individual has colorectal cancer.

Aspect 38. The method of any one of aspects 33-36, wherein the individual has metastatic colorectal cancer.

Aspect 39. The method of aspect 38 wherein the metastatic colorectal cancer has metastasized to the liver.

Aspect 40. A method of preventing or delaying the onset of cancer recurrence in an individual, comprising administering to the individual after one or more cancer therapies a therapeutically effective amount of one or more inhibitors of CSF-1 and/or one or more inhibitors of CSF-1R if the individual has ctDNA.

Aspect 41. The method of aspect 40, further comprising assaying for ctDNA in a sample from the individual.

Aspect 42. The method of aspect 41, wherein no ctDNA is detected in the individual.

Aspect 43. The method of aspect 41, wherein ctDNA is detectable in the individual.

Aspect 44. The method of any one of aspects 40-43, wherein the individual has colorectal cancer.

Aspect 45. The method of any one of aspects 40-44, wherein the individual has metastatic colorectal cancer.

Aspect 46. The method of aspect 45, wherein the metastatic colorectal cancer has metastasized to the liver.

It is specifically contemplated that any limitation discussed with respect to one embodiment or aspect of the disclosure may apply to any other embodiment or aspect of the disclosure. Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features, and advantages of the present disclosure will become apparent from the following detailed description. It should be understood, however, that the detailed description and the specific examples, while indicating specific embodiments or aspects of the disclosure, are given by way of illustration only, since various changes and modifications within the spirit and scope of the subject matter of the disclosure will become apparent to those skilled in the art from this detailed description.

### BRIEF DESCRIPTION OF THE DRAWINGS

The following drawings form part of the present specification and are included to further demonstrate certain aspects of the present disclosure. The invention may be better understood by reference to one or more of these drawings in combination with the detailed description of specific embodiments presented herein.

FIG. 1A and 1B show examples of inhibitors of CSF-1R.

FIG. 2. Schematic diagram that illustrates the microscopic disease left behind therapy; minimal residual disease (MRD).

FIGS. 3A-3M. Immune landscape of early liver metastases in an immunocompetent colorectal cancer model. (3A) Schematic of experimental work flow showing isolation of mouse colon, generation of colonic organoids that were genetically engineered using adeno-associated virus (AAV-9) infection, and injection of tumor organoid cell line collected from subcutaneous tumors into the spleens of C57BL/6J mice (see also FIG. 7). (3B) (upper left) H&E stain shows liver focal lesion from intrasplenic injections; (lower left) immunohistochemistry (IHC) stain shows N-cadherin (mesenchymal marker); (upper and lower middle& right) IHC staining shows CDX2, e-cadherin, GFP, and B-catenin. Scale bar, 200 μm. (3C) Structural multiplex immunofluorescence (IF) panel showing the composition and proliferation of murine micro-and macro-metastases to identify CD31, ARGI, CD45, vimentin, KI-67, and PANCK. Scale bar, 200 μm (see also FIG. 7). (3D-3E) Quantification analysis showing (3D) total CD45+ per mm2, and (3E) double positive markers KI67+PANCK+ per mm2 in micro-metastases (micro, ≤0.5 mm in diameter) versus macro-metastases (macro, >0.5 mm in diameter). (3F) (left) UMAP plot of immune cell types in micro-and macro-metastases; (right) Immune cell type proportions in micro-and macro-metastases. (3G) Differential expression of genes in micro-vs macro-metastases. scRNA seq data were analyzed from 12 mice. Cell type key reflects order from top to bottom of cell type bars. (3H) Macrophage multiplex IF 6-plex panel of murine (left) micro-metastases and (right) macro-metastases identify markers for PD-L1, ARGI, CD163, IBA-1, CD11B, and CD11C (see also FIG. 8). Scale bar, 200 μm. (3I-3K) Quantification analysis of (3I) a single positive marker (IBA-1+) per mm2; (3J) double positive marker (IBA-1+CD163+), and (3K) double positive marker (IBA-1+CD11C+) per mm2 in micro-metastasis versus macro-metastases. Structural and macrophage multiplex IF data panels were collected from 16 mice and analyzed by Mann-Whitney U test; p<0.05 indicates significant; ns=non-significant. Mean+SEM arc shown. (3L-3M) iStar analysis of (3L) murine and (3M) human micro-and macro-metastases showing macrophages and Treg expression. Letters indicate T: Tumor, S: Stroma, N: Necrosis, L: Normal Liver.

FIGS. 4A-4G. Unique macrophage clusters associated with MRD initiation and progression. (4A) Hallmark enrichment pathways analysis for micro-and macro-metastases. Data were analyzed from 12 mice. Micromet bars are to the right, and Macromet bars are to the left. (4B) UMAP plot showing five distinct macrophage clusters. (4C) Heat map showing the differential gene expression for the five macrophage clusters. (4D) Heat map showing hallmark enrichment pathways in micro-metastases compared to macro-metastases within each macrophage cluster, with further categorization by macrophage priming, activation, and resolution pathways. (4E) CytoSPACE quantification analysis showing the percentage of cell proportions of macrophage clusters in micro-and macro-metastases. Data were collected from 24 murine liver metastases and analyzed using two-way ANOVA test, *p<0.0001. Mean+SEM are shown. Micromet bars are on the left of the bar pairs, and Macromet bars are on the right of the bar pairs. (4F) iStar analysis of human micro and macro-metastases showing the expression of the CSF1R high macrophage cluster. Letters indicate T: Tumor, S: Stroma, N: Necrosis, L: Normal Liver. (4G) (upper) Immunohistochemistry stain showing the human CSF1R expression in one representative micro-metastases. (lower) Macrophage multiplex immunofluorescence (IF) 6-plex panel showing the high infiltration of macrophages (IBA-1+) and macrophages expressing CD163+. (upper and lower left) Scale bar: 100 μm, (upper and lower right) Scale bar: 200 μm.

FIGS. 5A-5J. Anti-CSF1R monotherapy eradicates micro-metastases in a MRD CRC model. (5A-5B) Survival experiment showing the efficacy of anti-CSF1R treatment on experimental metastases models harboring (5A) micro-metastases or (5B) macro-metastases: (upper panels) experimental treatment plan; (lower panels) Kaplan-Meier survival curve showing estimated survival after treatment with anti-CSF1R or vehicle. In 5A, 5B, 5C, and 5D, vehicle is the top horizontal bar, and Anti-CSF1R is the lower horizontal bar. (5C-5D) Schematic of experiment assessing response of liver lesions to anti-CSF1R treatment in mice harboring (5C) micro-metastases or (5D) macro-metastases. (5E-5F) (left) MRI analysis of mouse livers post-treatment initiation (liver focal lesions indicated by red dashed circles) and (right) liver gross images post 28 days of anti-CSF1R or vehicle treatment from mice harboring (5E) micro-metastases or (5F) macro-metastases. (5G-5H) H&E stain of mouse liver sections on day 0, 7, 14, and 28 post-treatment initiation of anti-CSF1R or vehicle in models harboring (5G) micro-metastases or (5H) macro-metastases. Scale bar, 200 um. (51-5J) Statistical analysis of (left) number of focal lesions and (right) area of foci on day 0, 7, 14, and 28 post-treatment initiation of anti-CSF1R or vehicle in models harboring (5I) micro-metastases (Control is the higher line for both panels) or (5J) macro-metastases (for the left panel, the control is the top line, and for the right panel the control is the line on top at 14 and 28 days). Data were collected from 58 mice (micro-metastases experiment) and 40 mice (macro-metastases experiment) and analyzed using Mann-Whitney U test, *p<0.05; ** p<0.01. Mean+SEM are shown.

FIGS. 6A-6H. Anti-CSFIR treatment modulates the immune tumor microenvironment of micro-metastases. (6A-6B) CytoSPACE analysis showing the efficacy of anti-CSF1R treatment in modulating the immune compartment of the tumor microenvironment; specifically, (upper left) (6A) proportions of different immune cell types on day 7 of vehicle treatment, (upper right) (6B) day 14 of vehicle treatment,, (lower left) (6A) day 7 of anti-CSF1R treatment, and (lower right) (6B) day 14 of anti-CSF1R treatment. Data were analyzed from 9 vehicle mice liver metastases and 4 anti-CSF1R treated mice liver metastases. (6C) Macrophage multiplex immunofluorescence (IF) 6-plex panel showing macrophages expressing CD163+ in mice treated with anti-CSF1R (left) or vehicle (right) (see also FIG. 10). (6D) Quantification analysis showing total number of macrophages expressing CD163+ per mm2 in mice treated with anti-CSF1R or vehicle. Data were collected from 116 lesions and analyzed by two-way ANOVA test; ****p<0.0001. Mean+SEM are shown. For the pairs of bars, Vehicle is the bar on the left. (6E) T cell multiplex IF 6-plex panel showing CD8+ T cell numbers in mice treated with (left) vehicle or (right) anti-CSF1R (see also FIG. 11). (6F) Quantification analysis showing number of non-exhausted CD8+ T cells per mm2 in mice treated with anti-CSF1R or vehicle. Data were collected from 116 lesions and analyzed by two-way ANOVA test; ****p<0.0001. Mean+SEM are shown. For the pairs of bars, Vehicle is the bar on the left. (6G) CytoSPACE quantification analysis showing macrophage clusters proportions at day 14 of anti-CSF1R or vehicle treatment in mice. Data were collected from 5 vehicle mice liver metastases and 2 anti-CSF1R treated mice liver metastases and analyzed by two-way ANOVA test; ****p<0.0001. Mean+SEM are shown. For the pairs of bars, Vehicle D14 is the bar on the left. (6H) Schematic depicting immune modulation of MRD in CRC.

FIGS. 7A-7I. Generation of genetically engineered murine colonic organoids recapitulating human mCRC; Related to FIG. 3. (7A) Schematic of our genetically engineered mouse model from which our organoids originated for recapitulating mCRC. (7B) Bright field images of mouse colonic organoids generated. Scale bar, 4 mm, 100 μm, 200 μm, respectively. (7C) AAV-9 construct design harboring sgRNAs targeting Apc and Tp53. Schematic depicting gene-editing of the organoids, the subsequent subcutaneous transplantation of the engineered organoids, and the genomic analysis of the tumors derived from the implanted engineered organoids. (7D) H&E stain shows (upper left) murine primary colon tumor from submucosal colonic injections, (upper right) spontaneous murine liver metastases from colonic injections, (lower right) murine liver metastases from intrasplenic injections resembling human liver metastases from metastatic colorectal cancer (lower left). Scale bar, 200 μm. (7E) (left) Representative MRI of mouse livers showing liver metastases (indicated by red arrows); (right) representative liver gross images each showing two liver focal lesions (indicated by red arrow). (7F) H&E stain shows liver focal lesion from intrasplenic injections. (upper and lower left) one representative liver section (scale bar, 2 mm), (upper right) micro-focal lesion with 0.2 mm in diameter (scale bar, 200 μm), and (right lower) macro-focal lesion with 1.2 mm in diameter (scale bar, 200 μm). (7G) Structural immunofluorescence (IF) panel showing single plex of the composition and proliferation of micro-and macro-metastases. (7H) Log graph showing at 0.5 mm in diameter the difference in CD45+ expression was maximized. (7I) Quantification analysis showing single positive marker of total tumor cells (PANCK+) per mm² in micro-and macro-metastases. Data were collected from 16 mice and analyzed by Mann-Whitney U test; p<0.05 indicates significance; ns=non-significant. Mean+SEM are shown.

FIGS. 8A-8L. Prominent macrophage population in early metastatic lesions; Related to FIG. 3. (8A) Schematic showing sample preparation for single RNA sequencing analysis. (8B) Macrophage multiplex IF 6-plex panel of (upper) micro-and (lower) macro-metastases Scale bar, 200 μm. (8C-8F) Quantification analysis of double positive markers (8C) IBA-1+ARG1+ (8E) IBA-1+CD11B+, (8F) IBA-1+PDL-1+ per mm2, and triple positive marker (8D) IBA-1+CD163+ARG1+ per mm2 in micro-metastasis and macro-metastases. Data were collected from 16 mice and analyzed by Mann-Whitney U test; p<0.05 indicates significance; ns=non-significant. Mean+SEM are shown. (8G) T cell multiplex immunofluorescence (IF) 6-plex panel of (upper) micro-and (lower) macro-metastases, identify markers for PD-1, CTLA-4, CD8, CD4, FOXP3, and ICOS. Scale bar, 200 μm. (8H-8L) Quantification analysis identifying single positive marker (8H) CD4+ and (8J) CD8+ per mm2; double positive markers (81) CD4+ FOXP3+ and (8K) CD4+PD1+; (8L) the ratio of CD8+ to CD4+FOXP3+ cells per mm2 in micro-metastasis and macro-metastases. Data were collected from 16 mice and analyzed by Mann-Whitney U test; p<0.05 indicates significance; ns=non-significant. Mean+SEM are shown.

FIGS. 9A-9F. High infiltration of macrophages in human micro-metastases. Related to FIG. 3. (9A) Schematic for the human liver metastases preparation. (9B) H&E stain shows human (upper) micro-metastases and (lower) macro-metastases. Scale bar, 200 μm. (9C) Macrophage multiplex IF 6-plex panel of (upper) micro-metastases and (lower) macro-metastases. Scale bar, 200 μm. (9D) Quantification analysis of double positive markers showing total number of macrophages expressing CD163+ (IBA-1+CD163+) per mm2, in micro-metastasis and macro-metastases. (9E) T cell multiplex IF 6-plex panel of (upper) micro-metastases and (lower) macro-metastases. Scale bar, 200 μm. (9F) Quantification analysis of double positive markers showing total number of Treg cells (CD4+FOXP3+) per mm2, in micro-metastasis and macro-metastases. Macrophage and T cell multiplex IF data panels were collected from 22 patients' metastases and analyzed by Mann-Whitney U test; p<0.05 indicates significance; ns=non-significant. Median with interquartile range is shown.

FIGS. 10A-10F. Anti-CSF1R treatment has no effect on dendritic cells expression in micro-metastases; Related to FIG. 6. (10A-10B) Macrophage multiplex IF 6-plex panel showing expression of macrophages from micro-metastases on day 0, 7, or 14 of (10A) vehicle or (10B) anti-CSF1R treatment. Scale bar, 200 μm. (10C-10F) Quantification analysis comparing (10C) single positive marker (IBA-1+) per mm2 as well as (10D-10F) double positive markers (10D) total number of dendritic cells (IBA-1+CD11C+) per mm2, (10E) total number of macrophages expressing ARG1+ (IBA-1+ARG1+) per mm2, and (10F) total number of bone marrow derived macrophages (IBA-1+CD11B+) per mm2) in response to anti-CSF1R or vehicle treatment. Data were collected from 116 lesions and analyzed by two-way ANOVA test; ns=non-significant. Mean+SEM are shown. For FIGS. 10C, 10D, 10E, and 10F, for the pairs of bars the Vehicle is the bar on the left.

FIGS. 11A-11F. Anti-CSF1R treatment has no effect on Treg cells expression in micro-metastases; Related to FIG. 6. (11A-11B) T cell multiplex immunofluorescence (IF) 6-plex panel showing expression of CD8+ T cells and CD4+FOXP3+ T cells from micro-metastases on day 0, 7, or 14 of (11A) vehicle or (11B) anti-CSF1R treatment. Scale bar, 200 μm. (11C-11F) Quantification analysis showing (11C) single positive markers, total number of conventional CD4+ T cells per mm2, as well as (11D-11F) double positive markers (11D) total number of Treg (CD4+FOXP3+) per mm2, (11E) total number of T conventional cells expressing PD1+ per mm2, and (11F) total number of T conventional cells expressing CTLA4+ per mm2) in micro-metastases from mice treated with anti-CSF1R or vehicle. Data were collected from 116 lesions and analyzed by two-way ANOVA test; ns=non-significant. Mean+SEM are shown. For FIGS. 11C, 11D, 11E, and 11F, for the pairs of bars the Vehicle is the bar on the left.

FIGS. 12A-12F. Anti-CSF1R treatment does not deplete macrophages expressing CD163+ in macro-metastases; Related to FIG. 6. (12A-12B) Macrophage multiplex immunofluorescence (IF) 6-plex panel showing expression of macrophages from macro-metastases on day 0, 7, or 14 of (12A) vehicle or (12B) anti-CSF1R treatment. Scale bar, 200 μm. (12C-12F) Quantification analysis showing (12C) single positive marker (IBA-1+) per mm2 as well as (12D-12F) double positive markers (12D) total number of macrophages expressing CD163+ (IBA-1+CD163+) per mm2, (12E) total number of dendritic cells (IBA-1+ CD11C+) per mm2, and (12F) total number of bone marrow derived macrophages (IBA-1+ CD11B+) per mm2) in macro-metastases from mice treated with anti-CSF1R or vehicle. Data were collected from 99 lesions and analyzed by two-way ANOVA test; ns=non-significant. Mean+SEM are shown.

FIGS. 13A-13D. Anti-CSF1R treatment does not enhance CD8+ T cells in macro-metastases; Related to FIG. 6. (13A-13B) T cell multiplex immunofluorescence (IF) 6-plex panel showing expression of CD8+ T cells and CD4+FOXP3+ T from macro-metastases on day 0, 7, or 14 of (13A) vehicle or (13B) anti-CSF1R treatment. Scale bar, 200 μm. (13C-13D), Quantification analysis showing double positive markers (13C) total number of non-exhausted CD8+ T cells per mm2 and (13D) total number of Treg (CD4+FOXP3+) per mm2 in macro-metastases from mice treated with anti-CSF1R or vehicle. Data were collected from 99 lesions and analyzed by two-way ANOVA test; **p<0.01; ****p<0.0001; ns=non-significant. Mean+SEM are shown. For FIGS. 13C and 13D, for the pairs of bars the Vehicle is the bar on the left.

### DETAILED DESCRIPTION

### I. EXAMPLES OF DEFINITIONS

It is contemplated that any embodiment discussed in this specification can be implemented with respect to any method or composition of the invention, and vice versa. Furthermore, compositions of the invention can be used to achieve methods of the invention.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this application, the term “about” is used to indicate that a value includes the inherent variation of error for the device, the method being employed to determine the value, or the variation that exists among the study subjects.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

“Subject” and “patient” and “individual” may be interchangeable and may refer to either a human or non-human, such as primates, mammals, and vertebrates. In particular embodiments, the subject is a human. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more cancers. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants. A subject may or may not have a need for medical treatment; an individual may voluntarily or involuntarily be part of experimentation whether clinical or in support of basic science studies. In specific embodiments, the individual has been treated for cancer and/or is being treated for cancer.

“Treating” or treatment of a disease or condition refers to executing a protocol, which may include administering one or more therapeutic agents to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, complete eradication of the tumor, a reduction in the size of a tumor, a reduction in the invasiveness of a tumor, reduction in the growth rate of the cancer, or prevention of metastasis. Treatment of cancer may also refer to prolonging survival of a subject with cancer.

The term “minimal residual disease (MRD)” as used herein refers to any residual or remaining cancer cells, after cancer treatment, in the body which may be below detection limit of conventional diagnostic tests such as by radiographic or histochemical detection, although DNA or proteins released from the cancer cells may provide alternative ways for detection. MRD is also referred to as measurable residual disease or molecular residual disease. Examples of assays for testing for MRD include at least multiparametric flow cytometry (MFC), quantitative polymerase chain reaction (qPCR), digital PCR (dPCR), next-generation sequencing (NGS), next-generation flow cytometry (NGF), RT-PCR, mass spectrometry, methylation analysis, hybrid sequencing (Nanostring), or a combination thereof. In some embodiments, MRD is the microscopic disease left behind after therapy. In particular embodiments, assays for testing for MRD may be a targeted approach that tests for known mutations and/or an untargeted approach that broadly tests for unknown targets. A targeted approach may include real-time polymerase chain reaction (RT-PCR), digital PCR (dPCR), and/or beads, emulsion, amplification, and magnetics (BEAMing) technology, whereas a broader approach would include high-throughput sequencing methods based on next-generation sequencing (NGS), whole exome sequencing (WES), whole genome sequencing (WGS), and/or mass-spectrometry-based detection of PCR amplicons.

### II. GENERAL ASPECTS

In certain aspects, the disclosure concerns treatment for cancer, including for MRD or reducing the risk for having MRD, including at least delaying the onset and/or severity of MRD, in some cases. In particular aspects, it is shown herein that one or more inhibitors of CSF-1 and/or one or more inhibitors of CSF-1R inhibit MRD in mammals. In embodiments of the disclosure, MRD of any cancer may be treated with one or more inhibitors of CSF-1 and/or one or more inhibitors of CSF-1R. In specific aspects, anti-CSFI/CSF1R therapy inhibits MRD, including as shown in a novel immunocompetent murine colorectal cancer metastasis model.

Colorectal cancer (CRC) is the second leading cause of cancer deaths recurrence because of the progression of MRD. One characteristic of MRD is the presence of micro-metastases, a potential indicator of persistent and/or recurrent malignancy before becoming clinically evident. Thus, because of its potential role as a precursor to metastatic cancer, clucidation into the immunobiology driving and enabling MRD progression is useful. Recent studies have suggested a role for the immune system in enabling the persistence of MRD, as the expansion of micro-metastatic lesions is subjected to environmental stresses that select for metastatic cells capable of escaping or neutralizing the cytotoxic activity of intrinsic and extrinsic immunosurveillance influences. However, the selective advantages that enable the initiation, stabilization, escape of micro-metastases from antitumor immunity, leading to the eventual growth into macro-metastases in the context of MRD and the eventual development of metastatic cancer, remain poorly understood. For instance, in the context of metastatic colorectal cancer (mCRC), CD3+ T cells that are generally immune-stimulating initially infiltrate in abundance into liver micro-metastases but progressively decline as micro-metastases develop into macro-metastases, thus suggesting that the induction of a suppressive liver microenvironment may favor tumor progression and is an evolving process. However, several immunosuppressive mechanisms induced by the dynamics of liver homeostasis may also enable tolerant immune responses from either T-regulatory cells (T-reg) or hepatic macrophages, which are composed of a heterogenous population of cells that include tissue-resident Kupffer cells and monocyte-derived macrophages. Therefore, it was considered that the activation of a suppressive immune population is a key mechanism that drives the initiation and progression of minimal residual disease of colorectal cancer.

To characterize this, genetically engineered mouse CRC organoids were generated to recapitulate human CRC. An experimental metastasis model was used in syngeneic, immunocompetent, C57BL/6 mice to generate hepatic CRC metastases. Differences in the immune infiltrates between micro-metastases that mimicked MRD and radiographically detectable macro-metastases were detected using multiplex immunofluorescent (IF) analysis from three different 6-marker panels.

In specific embodiments, a mouse colonic organoids model recapitulating mCRCs was generated to study the tumor immune microenvironment in minimal residual disease. Colonic organoids are established from CDX2 CRE; Rosa26 LSL-CAS9-GFP mice and grown as a heterogenous 3D culture until reaching sizes large enough to exhibit epithelial characteristics at the outer edges and a lumen. Adeno-associated virus-9 (AAV-9) particles harboring single guided RNAs (sgRNAs) of APC and TP53 tumor suppressor genes were then used to induce tissue-specific somatic gene editing in mice colonic organoids that expressed a conditional Cas9 allele. Tumor-bearing mice were confirmed by colonoscopy, and the characteristics of mCRC were confirmed in GE organoids through histopathological examination and whole exome sequencing.

The roles of immune cells in liver micro-and macro-metastasis are delineated. To determine whether liver macro-metastases may recruit immune suppressive populations that support growth, the inventors first identified and, using immunofluorescence (IF), delineated the immune cell compartments in liver micro-and macro-metastases in C57BL/6J mice that were injected with the GE organoids. To determine the similarities between the immune compartments of patient tumors and tumors from the syngeneic mice, the inventors used multiplex IF to analyze the expression of structural, T-regulatory, and macrophage panel markers on patients' micro-and macrometastases.

Finally, to identify subsets of immune cells and to confirm multiplex IF results, spatial transcriptomic analysis was performed using Visium technology and scRNA seq analysis. The findings allow for a comprehensive understanding of the contributions of immune cell populations to MRD of CRC.

The therapeutic efficacy of macrophage depletion on MRD progression was examined. The results showed that M2 macrophages are associated with progression of micro-metastases. Thus, in certain embodiments targeting M2 macrophages is a new therapeutic targeting of MRD. Agents that inhibit the activation, infiltration or activity, or promote the depletion, of M2 macrophages (“M2 macrophage-targeting agents”) are known in the art. It is known that the primary activation of M2 macrophages is caused by triggering cytokines, which include transforming growth factor (TGF)-B, IL-4, IL-13, IL-10, and macrophage colony-stimulating factor (M-CSF or CSF1R). M2-like macrophages highly express CD163, CD206, CD200R, CD209, CD301 and chemokines like CCL1, CCL17, CCL18, CCL22, and CCL24. They release numerous anti-inflammatory factors, including TGF-β, IL-4, IL-13, IL-10, and IL-IRA. Additionally, M2-like TAMs express the inflammatory cytokines IL-6, IL-12, IL-23 and TNF-α at lower levels. Inhibitors of CSF1/CSF1R, CLL2, SIRPα, TIE2, Arginase, HER2, GC vitamin D-binding protein, CD40, BTK, CD47, and CCR2 are being developed as M2 macrophage-targeting agents and thus are within the scope of the instant disclosure. Although many pathways exist to deplete macrophages, CSF1R is a primary receptor for macrophage survival and progression from M1 to M2 polarization. In diffuse-type tenosynovial giant cell tumor (dt-GCT) clinical trials, a tumor with high expression of CSF1, CSF1R inhibitor is strongly applied. To characterize the efficacy of targeting macrophages on micrometastases progression, the inventors used anti-CSF1R antibody “CD115” and control agent (rat Ig2a isotype) after 7 days of intrasplenic tumor injection. To investigate the efficacy of anti-CSF1R on macro-metastases regression, the mice were treated after 14 days post tumor injection. Results are confirmed using MRI and histopathology. This allows identification of the contribution of immune cell population in the initiation and progression of MRD mCRC and exploits the therapeutic targeting of those suppressive immune cells.

Thus, embodiments of the disclosure include uses of inhibitor(s) of CSF1R (also known as CSF-1R, colony stimulating factor 1 receptor, macrophage colony-stimulating factor 1 receptor, CD115 antigen, CD115, CSF-1 receptor, FMS proto-oncogene, McDonough feline sarcoma viral (v-fms) oncogene homolog, macrophage colony stimulating factor 1 receptor, and proto-oncogene c-Fms)). Aspects of the disclosure encompass anti-CSF-1 antibody monotherapy or anti-CSF-1R antibody monotherapy that inhibits minimal residual disease. Specific aspects encompass a novel immunocompetent murine colorectal cancer metastasis model.

In specific embodiments, circulating tumor DNA (ctDNA) patients have minimal residual disease (MRD). For the purpose of this disclosure the inventors coupled novel pre-clinical mouse models of MRD to identify and target macrophage populations expressing CSF-1R in immune-competent mice. Inhibitors of CSF-1R were effective in treating MRD in the mice. In specific embodiments, one can utilize an M2 macrophage-targeting agent toward a curative intent.

In some embodiments, an individual with ctDNA as a biomarker in serum are administered therapeutically effective amounts of one or more M2 macrophage-targeting agent. ctDNA enters the blood stream from tumors by a variety of mechanisms including: neoangiogenesis or the formation of new blood vessels, tumor cell death, tumor cell invasion into the blood stream, among others. Methods of the disclosure may include the step of detecting the presence of ctDNA, such as pre-radiographically detectable tumor following attempts at curative treatment. Once treatment is attempted, one can use workflows and approaches to detect ctDNA, such as when a patient returns for follow-up. In some embodiments, an individual is given the therapy in the absence of first detecting ctDNA. If ctDNA is present as a serum biomarker, there may be a high probability of recurrence of the patient cancer.

In specific embodiments, one can measure for the ctDNA over time. In some embodiments, this affords the opportunity to assess the presence of microscopic disease when undetectable, such as radiographically. The use of a serum-based ctDNA biomarker provides a cumulative indicator of the total microscopic burden of disease, in specific aspects. In some embodiments, this is a useful time for initiating additional treatment when the disease burden is the lowest level. It also may provide a start point for further monitoring of MRD as a function of ctDNA. In specific embodiments, one can use curative attempt at surgery as a start point for follow-up. In particular embodiments, most commonly ctDNA-patients indicates that disease is under control whereas ctDNA+ patients indicates that disease is still present or may be growing. The CSF-1 inhibitor(s) and/or CSF-1R inhibitor(s) may be given as part of maintenance therapy or preventatively to an individual that has received and/or is receiving therapy with curative intent. In some embodiments, the M2 macrophage-targeting agents (e.g., one or more inhibitors of CSF-1 or one or more CSF-1R inhibitors) are provided to an individual following a particular duration of time after cessation of the therapy with curative intent, such as within 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks or more, or within 1, 2, 3, 4, 5, 6, 7, 8, 9, 10, or more years following cessation of the therapy with curative intent. The therapy with curative intent may be surgery, radiation, chemotherapy, drug therapy, hormone therapy, immunotherapy, or a combination thereof. The therapy may be local or systemic. In some embodiments, the therapy is for colorectal cancer, such as polypectomy, surgical resection, J-Pouch Surgery, 5-Fluorouracil (5-FU), Capecitabine, Irinotecan, Oxaliplatin, Trifluridine and tipiracil, Bevacizumab, Ramucirumab, Ziv-aflibercept, Fruquintinib, Cetuximab, Panitumumab, Encorafenib, Trastuzumab, Pertuzumab, Tucatinib, Lapatinib, Fam-trastuzumab deruxtecan, Larotrectinib, entrectinib, Selpercatinib, Adagrasib, Sotorasib, or a combination thereof.

Since this is the lowest disease burden time point, one can target any immunosuppressive mechanisms that allow for regrowth of the tumors. In accordance with one embodiment of the present disclosure, provided is a method for treating MRD in a patient in need thereof. In some embodiments, the treatment entails administration of an M2 macrophage-targeting agents (e.g., one or more inhibitors of CSF-1 or one or more CSF-1R inhibitors).

In some embodiments, the patient does not have radiographically detectable cancer cells. In some embodiments, the patient has been treated for the cancer and is considered cancer free radiographically. In some embodiments, the patient has been diagnosed as having MRD based on the presence of circulating tumor DNA (ctDNA) or protein. In some embodiments, the patient has been treated for the cancer and has no detected ctDNA or circulating tumor protein, but is considered as at risk of tumor recurrence. In some embodiments, no cancer recurrence is expected, and the treatment is administered preemptively or preventively, or as a maintenance therapy.

In some embodiments, the MRD is derived from a cancer that is at relatively high risk of cancer recurrence following initial treatments. In some embodiments, the MRD is derived from a tumor that is considered a “cold tumor” or “nonimmunogenic tumor” that may be resistant to one or more immune checkpoint blockade (ICB) therapies.

Identification of nonimmunogenic, or cold tumors can be made with measurements of type, density and location of immune cells within the tumors. For instance, Galon and Bruni (Nature Reviews Drug Discovery volume 18, pages 197-218 (2019)) describes a standardized scoring system, Immunoscore, based on the quantification of two lymphocyte populations (CD3 and CD8), e.g., in resected tissues, for guided stratification of hot and cold tumors. The Immunoscore ranges from Immunoscore 0 (10, for low densities, such as absence of both cell types in both regions) to 14 (high immune cell densities in both locations). By classifying cancers according to their immune infiltration, the scoring system provides an immune-based classification of tumors, including a definition of “hot” (highly infiltrated, Immunoscore I4) and “cold” (non-infiltrated, Immunoscore 10) tumors.

In some embodiments, the cancer has an 10 immunoscore. In some embodiments, the cancer has an Il immunoscore. In some embodiments, the cancer has an 12 immunoscore. In some embodiments, the cancer has an 13 immunoscore.

In some embodiments, the cancer has one or more p53 mutation or B-catenin mutation, or high microsatellite instability (MSI-high). MSI-High (MSI-H) is a classification used in cancer diagnosis. It refers to tumors with significant microsatellite instability.

In some embodiments, the cancer is classified as microsatellite stable (MSS). In some embodiments, the cancer is MSS CRC.

In some embodiments, the patient has been treated with one or more immune checkpoint blockade (ICB) therapies, such as an anti-PD-L1 and/or anti-PD-1 treatment. In some embodiments, the patient is resistant to such a treatment.

To establish an example of a model for MRD, one can use a splenic injection approach to establish tumors in the livers of wild type mice by first injecting a novel organoid derived cell line that incorporated a somatic mutation approach to eliminate two immunosuppressor genes Apc and p53 that allows the cells to become tumorigenic. Organoids are generated from the colon tissue of genetically engineered to express a colon promoter specific Cdx2 Cre that occurs primarily in the distal colon. They co-express a Rosa26 ubiquitous promoter driven Lox-Stop-Lox, that once excised from by Cre expresses Cas9 and GFP where the tissue specific excision occurred. Organoids are generated and an adenovirus may be used to infect and eliminate APC and p53 with guide RNAs in a tissue specific fashion due to the previous steps. Lentiviral vectors may be used to express KRAS and BRAF using puro selection.

Tumorgenicity was confirmed by injecting subcutaneously into C57/BL6 NSG. Tumors were then used to establish organoids that were monodispersed and injected by a variety of routes that maintained tumorgenicity. Using these organoid cell lines the splenic injection model was used to establish a single start point and timing for treatments at micromet 7 days stage of growth (lesions that are ≤0.5 mm in size) or at macromet 21 days (lesions that are ≥1.2 mm in size) in C57BL6 wt mice. Treatment with an over-the-counter rat anti-mouse-CSF-1R monoclonal from BioXCell at 7 days when lesions were undetectable by MRI led to a complete cure. Treatment with the BioXCell an over rat anti-mouse-CSF-1R monoclonal from at 21 days when the liver lesions were detectable by MRI failed to result in a cure.

The inventors have extensive pre-clinical data indicating that when disease is microscopic that subsets of macrophages that are IBA1+CD163+ by multi-plex immunofluorescence using a novel panel of antibodies that recognize both human and mouse epitopes for cross comparison (M2 immunosuppressive macrophages by some nomenclatures). This macrophage subset falls into a cluster group as C6 defined by spatial transcriptomics as Apoe, CSF-1R, CCR5 expressing macrophages. In specific embodiments, CSF-1R expressing macrophages are targeted using a suitable inhibitor, including, e.g., the AmMax Bio, AMB001 anti-CSF-1R human antibody. One can also use the M279 rat anti-mouse-CSF-1R monoclonal antibody as an example to target these subsets of M2 immunosuppressive macrophages. The inventors have seen the conversion of the immunosuppressive macrophages to IBA1+CD11C+ or a pro-inflammatory macrophage subset (M1 pro-inflammatory macrophages) by multi-plex immunofluorescence using a novel anti-mouse/human antibody panel. This macrophage subset falls into a cluster group as C11 defined by spatial transcriptomics as Ace, Itgal, PlacB expressing macrophages. As an overall consideration, the use of timed M279 anti-CSF-1R treatment in mice at microscopic/pre-radiographically detectable tumor stage leads to a complete regression and cure that can be recapitulated in ctDNA+MRD patients using AmMax Bio, AMB001 anti-CSF-1R humanized antibody. As a mechanistic basis, in some embodiments this treatment involves the conversion of immunosuppressive macrophage subsets to proinflammatory macrophage subsets that eliminate MRD.

### III. SPECIFIC ASPECTS

Aspects of the disclosure include ctDNA+ patient workflow and approaches to identify patients with minimal residual disease to employ an M2 macrophage-targeting agent, such as an inhibitor of CSF-1 and/or CSF-1R. In some aspects, an anti-CSF-1R antibody is employed to treat an individual.

In particular embodiments, ctDNA+ MRD patients are treated using AmMax Bio, AMB001 anti-CSF-1R human antibody that provides a complete response as a single agent. In some embodiments, in the event that resistance to therapy occurs, an anti-CSF-1R antibody such as AMB001 can be used in combination with other anti-cancer agents if tumors become resistant to therapy. These combination use cases might include (in some embodiments): 1) Co-targeting oncogenic driver mutations such as p53, APC, Kras, Braf. 2) Targets of apoptosis evasion, such as one or more of the BCL family of proteins including BCL-2, Bcl-xL, Mcl-1, Bcl-w, and Bcl-B, p53 in combination with AMB001. 3) Targeting methylation and miss match DNA repair along with AMB001. 4) Combination of AMB001 with other immunotherapy for example: a) Immune checkpoints: PD1, PD-L1, CTLA-4, b) Antibody drug conjugates and bispecific T-cell engaging therapy, and c) C-type lectin-like molecule-1 (CLL1 or CLEC12A). 5. Chimeric antigen receptor therapy combination with targeting cellular therapy targets such as, CAR-T cells (TGFbetaR, Fucosylated) or CAR-NK cells (Trop2). 6. Combination AMB001 with agents that effect T-reg and/or dendritic cells.

The unique combination of ctDNA-guided MRD detection workflows, datasets, samples and clinical experience along with motivated patients provide platform for using inhibitors of CSF-1 and/or CSF-1R, including AmMax Bio, an AMB001 anti-CSF-1R humanized antibody. Highlighting a mechanistic basis for this anti-CSF-1R treatment, studies show that this treatment involves the plasticity state conversions in the immuno-microenvironment evolves from being dominated by immunosuppressive macrophage subsets to proinflammatory macrophage subsets that eliminate MRD. ctDNA+ patients receive this treatment with curative intent.

### IV. INHIBITORS OF CSF-1 OR CSF-1R

In particular embodiments, one or more inhibitors of CSF-1 and/or one or more inhibitors of CSF-1R are utilized to treat cancer of any kind, including MRD of any kind. The cancer or MRD may or may not be metastatic. The CSF-1 inhibitor(s) and/or CSF-1R inhibitor(s) may be a small molecule, a protein (e.g., a ligand of the receptor), a nucleic acid, a cell comprising the inhibitor, or a combination or mixture thereof.

In specific aspects, the inhibitor may be a kinase inhibitor, an antibody, a CSF1 ligand, a DNA inhibitor, an RNA inhibitor, an immune effector cell with a CSF-1R-specific chimeric antigen receptor, or a combination or mixture thereof.

### A. CSF-1 Inhibitors

In specific aspects, the inhibitor inhibits the CSF-1 protein directly or indirectly. A CSF-1 sequence is located at GenBank® Accession No. NP_000748.4, which is as follows:

(SEQ ID NO: 18)
MTAPGAAGRCPPTTWLGSLLLLVCLLASRSITEEVSEYCSHMIGSGHL
QSLQRLIDSQMETSCQITFEFVDQEQLKDPVCYLKKAFLLVQDIMEDT
MRFRDNTPNAIAIVQLQELSLRLKSCFTKDYEEHDKACVRTFYETPLQ
LLEKVKNVFNETKNLLDKDWNIFSKNCNNSFAECSSQDVVTKPDCNCL
YPKAIPSSDPASVSPHQPLAPSMAPVAGLTWEDSEGTEGSSLLPGEQP
LHTVDPGSAKQRPPRSTCQSFEPPETPVVKDSTIGGSPQPRPSVGAFN
PGMEDILDSAMGTNWVPEEASGEASEIPVPQGTELSPSRPGGGSMQTE
PARPSNFLSASSPLPASAKGQQPADVTGTALPRVGPVRPTGQDWNHTP
QKTDHPSALLRDPPEPGSPRISSLRPQGLSNPSTLSAQPQLSRSHSSG
SVLPLGELEGRRSTRDRRSPAEPEGGPASEGAARPLPRFNSVPLTDTG
HERQSEGSFSPQLQESVFHLLVPSVILVLLAVGGLLFYRWRRRSHQEP
QRADSPLEQPEGSPLTQDDRQVELPV

In some embodiments, the CSF-1 inhibitor is MCS110 (Novartis Pharmaceuticals) and/or PD0360324 (M.D. Anderson Cancer Center and Pfizer).

In addition, or alternative to, other CSF-1 inhibitors, the inhibitor may be a nucleic acid that inhibits expression (fully or partially). In specific embodiments, the inhibitor inhibits (in part or entirely) the nucleic acid sequence of GenBank® Accession No. NM_000757, which is as follows:

| (SEQ ID NO: 19) | |
|---|---|
| 1 | gaggctcggc ccggggaaag tgaaagtttg cctgggtcct ctcggcgcca gagccgctct |
| 61 | ccgcatccca ggacagcggt gcggccctcg gccggggcgc ccactccgca gcagccagcg |
| 121 | agcgagcgag cgagcgaggg cggccgacgc gcccggccgg gacccagctg cccgtatgac |
| 181 | cgcgccgggc gccgccgggc gctgccctcc cacgacatgg ctgggctccc tgctgttgtt |
| 241 | ggtctgtctc ctggcgagca ggagtatcac cgaggaggtg tcggagtact gtagccacat |
| 301 | gattgggagt ggacacctgc agtctctgca gcggctgatt gacagtcaga tggagacctc |
| 361 | gtgccaaatt acatttgagt ttgtagacca ggaacagttg aaagatccag tgtgctacct |
| 421 | taagaaggca tttctcctgg tacaagacat aatggaggac accatgcgct tcagagataa |
| 481 | cacccccaat gccatcgcca ttgtgcagct gcaggaactc tctttgaggc tgaagagctg |
| 541 | cttcaccaag gattatgaag agcatgacaa ggcctgcgtc cgaactttct atgagacacc |
| 601 | tctccagttg ctggagaagg tcaagaatgt ctttaatgaa acaaagaatc tccttgacaa |
| 661 | ggactggaat attttcagca agaactgcaa caacagcttt gctgaatgct ccagccaaga |
| 721 | tgtggtgacc aagcctgatt gcaactgcct gtaccccaaa gccatcccta gcagtgaccc |
| 781 | ggcctctgtc tcccctcatc agcccctcgc cccctccatg gcccctgtgg ctggcttgac |
| 841 | ctgggaggac tctgagggaa ctgagggcag ctccctcttg cctggtgagc agcccctgca |
| 901 | cacagtggat ccaggcagtg ccaagcagcg gccacccagg agcacctgcc agagctttga |
| 961 | gccgccagag accccagttg tcaaggacag caccatcggt ggctcaccac agcctcgccc |
| 1021 | ctctgtcggg gccttcaacc ccgggatgga ggatattctt gactctgcaa tgggcactaa |
| 1081 | ttgggtccca gaagaagcct ctggagaggc cagtgagatt cccgtacccc aagggacaga |
| 1141 | gctttccccc tccaggccag gagggggcag catgcagaca gagcccgcca gacccagcaa |
| 1201 | cttcctctca gcatcttctc cactccctgc atcagcaaag ggccaacagc cggcagatgt |
| 1261 | aactggtacc gccttgccca gggtgggccc cgtgaggccc actggccagg actggaatca |
| 1321 | caccccccag aagacagacc atccatctgc cctgctcaga gaccccccgg agccaggctc |
| 1381 | tcccaggatc tcatcactgc gcccccaggg cctcagcaac ccctccaccc tctctgctca |
| 1441 | gccacagctt tccagaagcc actcctcggg cagcgtgctg ccccttgggg agctggaggg |
| 1501 | caggaggagc accagggatc ggaggagccc cgcagagcca gaaggaggac cagcaagtga |
| 1561 | aggggcagcc aggcccctgc cccgttttaa ctccgttcct ttgactgaca caggccatga |
| 1621 | gaggcagtcc gagggatcct tcagcccgca gctccaggag tctgtcttcc acctgctggt |
| 1681 | gcccagtgtc atcctggtct tgctggccgt cggaggcctc ttgttctaca ggtggaggcg |
| 1741 | gcggagccat caagagcctc agagagcgga ttctcccttg gagcaaccag agggcagccc |
| 1801 | cctgactcag gatgacagac aggtggaact gccagtgtag agggaattct aagctggacg |
| 1861 | cacagaacag tctctccgtg ggaggagaca ttatggggcg tccaccacca cccctccctg |
| 1921 | gccatcctcc tggaatgtgg tctgccctcc accagagctc ctgcctgcca ggactggacc |
| 1981 | agagcagcca ggctggggcc cctctgtctc aacccgcaga cccttgactg aatgagagag |
| 2041 | gccagaggat gctccccatg ctgccactat ttattgtgag ccctggaggc tcccatgtgc |
| 2101 | ttgaggaagg ctggtgagcc cggctcagga ccctcttccc tcaggggctg caccctcctc |
| 2161 | tcactccctt ccatgccgga acccaggcca gggacccacc ggcctgtggt ttgtgggaaa |
| 2221 | gcagggtgga cgctgaggag tgaaagaacc ctgcacccag agggcctgcc tggtgccaag |
| 2281 | gtatcccagc ctggacaggc atggacctgt ctccagagag aggagcctga agttcgtggg |
| 2341 | gcgggacagc gtcggcctga tttcccgtaa aggtgtgcag cctgagagac gggaagagga |
| 2401 | ggcctctgga cctgctggtc tgcactgaca gcctgaaggg tctacaccct cggctcacct |
| 2461 | aagtgccctg tgctggttgc caggcgcaga ggggaggcca gccctgccct caggacctgc |
| 2521 | ctgacctgcc agtgatgcca agagggggat caagcactgg cctctgcccc tcctccttcc |
| 2581 | agcacctgcc agagcttctc caggaggcca agcagaggct cccctcatga aggaagccat |
| 2641 | tgcactgtga acactgtacc tgcctgctga acagcctgcc cccgtccatc catgagccag |
| 2701 | catccgtccg tcctccactc tccagcctct ccccagcctc ctgcactgag ctggcctcac |
| 2761 | cagtcgactg agggagcccc tcagccctga ccttctcctg acctggcctt tgactccccg |
| 2821 | gagtggagtg gggtgggaga acctcctggg ccgccagcca gagccggtct ttaggctgtg |
| 2881 | ttgttcgccc aggtttctgc atcttgcact ttgacattcc caagagggaa gggactagtg |
| 2941 | ggagagagca agggagggga gggcacagac agagaggcta cagggcgagc tctgactgaa |
| 3001 | gatgggcctt tgaaatatag gtatgcacct gaggttgggg gagggtctgc actcccaaac |
| 3061 | cccagcgcag tgtcctttcc ctgctgccga caggaacctg gggctgaaca ggttatccct |
| 3121 | gtcaggagcc ctggactggg ctgcatctca gccccacctg catggtatcc agctcccatc |
| 3181 | cacttctcac ccttctttcc tcctgacctt ggtcagcagt gatgacctcc aactctcacc |
| 3241 | caccccctct accatcacct ctaaccaggc aagccagggt gggagagcaa tcaggagagc |
| 3301 | caggcctcag cttccaatgc ctggagggcc tccactttgt ggccagcctg tggtggtggc |
| 3361 | tctgaggcct aggcaacgag cgacagggct gccagttgcc cctgggttcc tttgtgctgc |
| 3421 | tgtgtgcctc ctctcctgcc gccctttgtc ctccgctaag agaccctgcc ctacctggcc |
| 3481 | gctgggcccc gtgactttcc cttcctgccc aggaaagtga gggtcggctg gccccacctt |
| 3541 | ccctgtcctg atgccgacag cttagggaag ggcagtgaac ttgcatatgg ggcttagcct |
| 3601 | tctagtcaca gcctctatat ttgatgctag aaaacacata tttttaaatg gaagaaaaat |
| 3661 | aaaaaggcat tcccccttca tccccctacc ttaaacatat aatattttaa aggtcaaaaa |
| 3721 | agcaatccaa cccactgcag aagctctttt tgagcacttg gtggcatcag agcaggagga |
| 3781 | gccccagagc cacctctggt gtccccccag gctacctgct caggaacccc ttctgttctc |
| 3841 | tgagaagtca agagaggaca ttggctcacg cactgtgaga ttttgttttt atacttggaa |
| 3901 | gtggtgaatt attttatata aagtcattta aatatctatt taaaagatag gaagctgctt |
| 3961 | atatatttaa taataaaaga agtgcacaag ctgc |

### B. CSF-1R Inhibitors

In specific aspects. the inhibitor inhibits the CSF-1R protein, directly or indirectly. A CSF-1R sequence is located at GenBank® Accession No. NP_005202.2, which is as follows:

(SEQ ID NO: 1)
MGPGVLLLLLVATAWHGQGIPVIEPSVPELVVKPGATVTLRCVGNGSV
EWDGPPSPHWTLYSDGSSSILSTNNATFQNTGTYRCTEPGDPLGGSAA
IHLYVKDPARPWNVLAQEVVVFEDQDALLPCLLTDPVLEAGVSLVRVR
GRPLMRHTNYSFSPWHGFTIHRAKFIQSQDYQCSALMGGRKVMSISIR
LKVQKVIPGPPALTLVPAELVRIRGEAAQIVCSASSVDVNFDVFLQHN
NTKLAIPQQSDFHNNRYQKVLTLNLDQVDFQHAGNYSCVASNVQGKHS
TSMFFRVVESAYLNLSSEQNLIQEVTVGEGLNLKVMVEAYPGLQGFNW
TYLGPFSDHQPEPKLANATTKDTYRHTFTLSLPRLKPSEAGRYSFLAR
NPGGWRALTFELTLRYPPEVSVIWTFINGSGTLLCAASGYPQPNVTWL
QCSGHTDRCDEAQVLQVWDDPYPEVLSQEPFHKVTVQSLLTVETLEHN
QTYECRAHNSVGSGSWAFIPISAGAHTHPPDEFLFTPVVVACMSIMAL
LLLLLLLLLYKYKQKPKYQVRWKIIESYEGNSYTFIDPTQLPYNEKWE
FPRNNLQFGKTLGAGAFGKVVEATAFGLGKEDAVLKVAVKMLKSTAHA
DEKEALMSELKIMSHLGQHENIVNLLGACTHGGPVLVITEYCCYGDLL
NFLRRKAEAMLGPSLSPGQDPEGGVDYKNIHLEKKYVRRDSGFSSQGV
DTYVEMRPVSTSSNDSFSEQDLDKEDGRPLELRDLLHFSSQVAQGMAF
LASKNCIHRDVAARNVLLTNGHVAKIGDFGLARDIMNDSNYIVKGNAR
LPVKWMAPESIFDCVYTVQSDVWSYGILLWEIFSLGLNPYPGILVNSK
FYKLVKDGYQMAQPAFAPKNIYSIMQACWALEPTHRPTFQQICSFLQE
QAQEDRRERDYTNLPSSSRSGGSGSSSSELEEESSSEHLTCCEQGDIA
QPLLQPNNYQFC

In aspects wherein the inhibitor is a small molecule kinase inhibitor, the inhibitor may be one or more of the following:

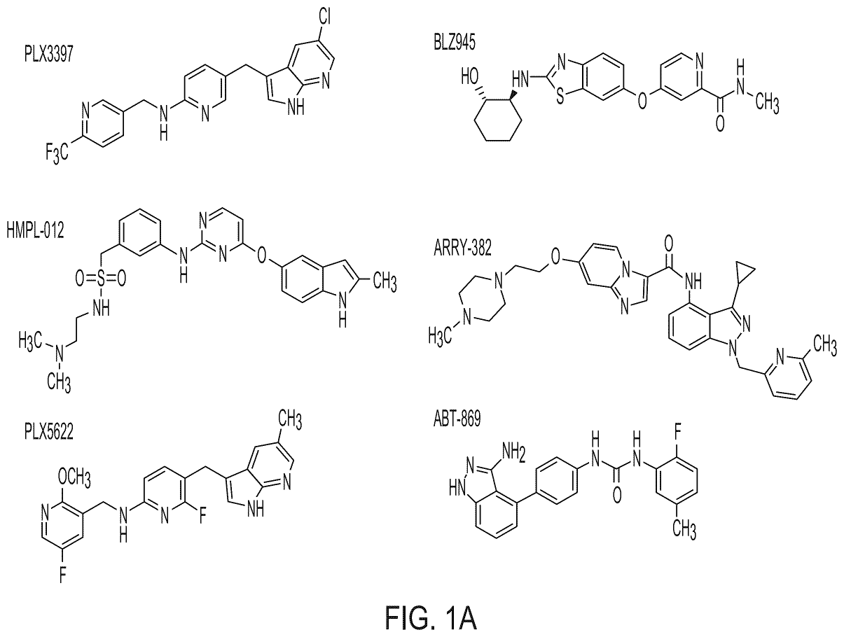

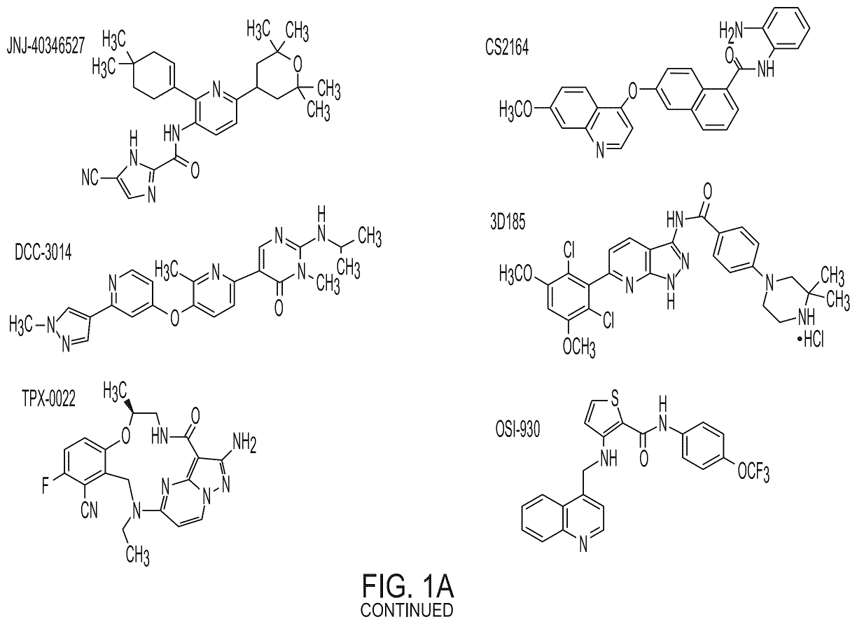

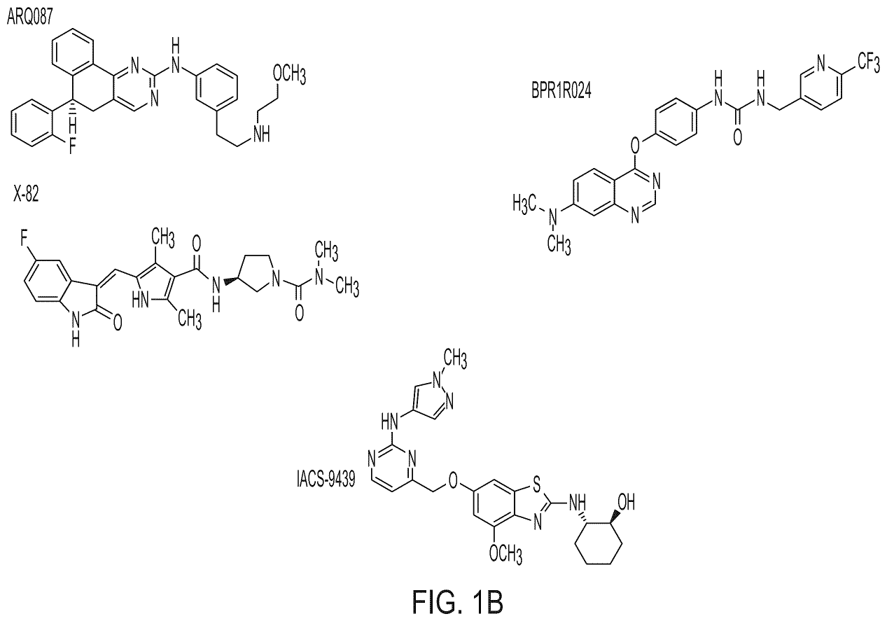

PLX3397 (Diachi/Plexicon); HMPL-012 (Nucleus Global, Shanghai, China, and Hutchison MediPharma); PLX5622, PLX73086, PLX7486 (Plexxikon); JNJ-40346527 (M.D. Anderson Cancer Center; OHSU Knight Cancer Institute; Johnson & Johnson Pharma; Janssen Research; and Provention Bio, Inc.); BLZ945 (both from Novartis); ARRY-382 (Pfizer and Array Biopharma); ABT-869 (Abbott and AbbVie); DCC-3014 (Deciphera); CS2164 (Chipscreen Biosciences, Ltd.) 3D185 (3D Medicines (Beijing) Co., Ltd.; NMS-03592088 (Nerviano Medical Sciences); OSI-930 (Astellas Pharma Inc.); Q702 (Qurient Co., Ltd.); TPX-0022 (Turning Point Therapeutics, Inc.); ARQ087 (Inst Klinische Krebsforschung, Krankenhaus Nordwest, and Basilea Pharmaceutica); X-82 (Tyrogenex, AnewPharma, Washington U School Medicine, and U Wisconsin, Madison); OPN-7486 (OpnaBio); IACS-9439 (M.D. Anderson Cancer Center and IACS); BPR1R024 (National Health Research Institutes, Taiwan); and/or ABSK021 (Abbisko and Merk KGaA) (see FIGS. 1A and 1B).

In addition, or alternative to, other types of CSF-1R inhibitors, the inhibitor may be one or more of the following:

AMB001; FPA008 (Five Prime Therapeutics, Inc., Bristol-Meyers Squibb, and Ono Pharmaceutical Co. Ltd.); LY3022855 (Eli Lilly and Company); RG7155 (SynOx Therapeutics Limited, M.D. Anderson Cancer Center, and Hoffmann-La Roche); and/or SNDX6532 (University of Utah Incyte Corporation and Syndax Pharma|AstraZeneca-Hopkins).

In specific embodiments, the CSF-1R inhibitor is, AMB001, an anti-CSF-1R human antibody and in particular embodiments it is used to target MRD in ctDNA+ CRC posttreatment. Cabiralizumab, LY3022855, and/or Emactuzumab may be utilized, in some aspects.

In some embodiments, the CSF-1R inhibitor is part of a chimeric antigen receptor (CAR) in which the extracellular domain is a CSF-1R antibody. The CAR may be in any type of immune effector cells, such as T cells, NK cells, NKT cells, dendritic cells, macrophages, etc. The CAR may have any number of costimulatory domains, including any type.

In addition, or alternative to, other types of CSF-1R inhibitors, the inhibitor may be a nucleic acid that inhibits expression (fully or partially). In specific embodiments, the inhibitor inhibits (in part or entirely) the nucleic acid sequence of GenBank® Accession No. NM_005211, which is as follows:

| (SEQ ID NO: 2) | |
|---|---|
| 1 | agacagagtg tccaaaagcg tgagagcacg aagtgaggag aaggtggaga agagagaaga |
| 61 | ggaagaggaa gaggaagaga ggaagcggag ggaactgcgg ccaggctaaa aggggaagaa |
| 121 | gaggatcagc ccaaggagga ggaagaggaa aacaagacaa acagccagtg cagaggagag |
| 181 | gaacgtgtgt ccagtgtccc gatccctgcg gagctagtag ctgagagctc tgtgccctgg |
| 241 | gcaccttgca gccctgcacc tgcctgccac ttccccaccg aggccatggg cccaggagtt |
| 301 | ctgctgctcc tgctggtggc cacagcttgg catggtcagg gaatcccagt gatagagccc |
| 361 | agtgtccctg agctggtcgt gaagccagga gcaacggtga ccttgcgatg tgtgggcaat |
| 421 | ggcagcgtgg aatgggatgg ccccccatca cctcactgga ccctgtactc tgatggctcc |
| 481 | agcagcatcc tcagcaccaa caacgctacc ttccaaaaca cggggaccta tcgctgcact |
| 541 | gagcctggag accccctggg aggcagcgcc gccatccacc tctatgtcaa agaccctgcc |
| 601 | cggccctgga acgtgctagc acaggaggtg gtcgtgttcg aggaccagga cgcactactg |
| 661 | ccctgtctgc tcacagaccc ggtgctggaa gcaggcgtct cgctggtgcg tgtgcgtggc |
| 721 | cggcccctca tgcgccacac caactactcc ttctcgccct ggcatggctt caccatccac |
| 781 | agggccaagt tcattcagag ccaggactat caatgcagtg ccctgatggg tggcaggaag |
| 841 | gtgatgtcca tcagcatccg gctgaaagtg cagaaagtca tcccagggcc cccagccttg |
| 901 | acactggtgc ctgcagagct ggtgcggatt cgaggggagg ctgcccagat cgtgtgctca |
| 961 | gccagcagcg ttgatgttaa ctttgatgtc ttcctccaac acaacaacac caagctcgca |
| 1021 | atccctcaac aatctgactt tcataataac cgttaccaaa aagtcctgac cctcaacctc |
| 1081 | gatcaagtag atttccaaca tgccggcaac tactcctgcg tggccagcaa cgtgcagggc |
| 1141 | aagcactcca cctccatgtt cttccgggtg gtagagagtg cctacttgaa cttgagctct |
| 1201 | gagcagaacc tcatccagga ggtgaccgtg ggggaggggc tcaacctcaa gaggcctacc |
| 1261 | gaggcctacc caggcctgca aggttttaac tggacctacc tgggaccctt ttctgaccac |
| 1321 | cagcctgagc ccaagcttgc taatgctacc accaaggaca catacaggca caccttcacc |
| 1381 | ctctctctgc cccgcctgaa gccctctgag gctggccgct actccttcct ggccagaaac |
| 1441 | ccaggaggct ggagagctct gacgtttgag ctcacccttc gatacccccc agaggtaagc |
| 1501 | gtcatatgga cattcatcaa cggctctggc acccttttgt gtgctgcctc tgggtacccc |
| 1561 | cagcccaacg tgacatggct gcagtgcagt ggccacactg ataggtgtga tgaggcccaa |
| 1621 | gtgctgcagg tctgggatga cccataccct gaggtcctga gccaggagcc cttccacaag |
| 1681 | gtgacggtgc agagcctgct gactgttgag accttagagc acaaccaaac ctacgagtgc |
| 1741 | agggcccaca acagcgtggg gagtggctcc tgggccttca tacccatctc tgcaggagcc |
| 1801 | cacacgcatc ccccggatga gttcctcttc acaccagtgg tggtcgcctg catgtccatc |
| 1861 | atggccttgc tgctgctgct gctcctgctg ctattgtaca agtataagca gaagcccaag |
| 1921 | taccaggtcc gctggaagat catcgagagc tatgagggca acagttatac tttcatcgac |
| 1981 | cccacgcagc tgccttacaa cgagaagtgg gagttccccc ggaacaacct gcagtttggt |
| 2041 | aagaccctcg gagctggagc ctttgggaag gtggtggagg ccacggcctt tggtctgggc |
| 2101 | aaggaggatg ctgtcctgaa ggtggctgtg aagatgctga agtccacggc ccatgctgat |
| 2161 | gagaaggagg ccctcatgtc cgagctgaag atcatgagcc acctgggcca gcacgagaac |
| 2221 | atcgtcaacc ttctgggagc ctgtacccat ggaggccctg tactggtcat cacggagtac |
| 2281 | tgttgctatg gcgacctgct caactttctg cgaaggaagg ctgaggccat agcctgagcc |
| 2341 | agcctgagcc ccggccagga ccccgaggga ggcgtcgact ataagaacat ccacctcgag |
| 2401 | aagaaatatg tccgcaggga cagtggcttc tccagccagg gtgtggacac ctatgtggag |
| 2461 | atgaggcctg tctccacttc ttcaaatgac tccttctctg agcaagacct ggacaaggag |
| 2521 | gatggacggc ccctggagct ccgggacctg cttcacttct ccagccaagt agcccagggc |
| 2581 | atggccttcc tcgcttccaa gaattgcatc caccgggacg tggcagcgcg taacgtgctg |
| 2641 | ttgaccaatg gtcatgtggc caagattggg gacttcgggc tggctaggga catcatgaat |
| 2701 | gactccaact acattgtcaa gggcaatgcc cgcctgcctg tgaagtggat ggccccagag |
| 2761 | agcatctttg actgtgtcta cacggttcag agcgacgtct ggtcctatgg catcctcctc |
| 2821 | tgggagatct tctcacttgg gctgaatccc taccctggca tcctggtgaa cagcaagttc |
| 2881 | tataaactgg tgaaggatgg ataccaaatg gcccagcctg catttgcccc aaagaatata |
| 2941 | tacagcatca tgcaggcctg ctgggccttg gagcccaccc acagacccac cttccagcag |
| 3001 | atctgctcct tccttcagga gcaggcccaa gaggacagga gagagcggga ctataccaat |
| 3061 | ctgccgagca gcagcagaag cggtggcagc ggcagcagca gcagtgagct ggaggaggag |
| 3121 | agctctagtg agcacctgac ctgctgcgag caaggggata tcgcccagcc cttgctgcag |
| 3181 | cccaacaact atcagttctg ctgaggagtt gacgacaggg agtaccactc tcccctccca |
| 3241 | caaacttcaa ctcctccatg gatggggcga cacggggaga acatacaaac tctgccttcg |
| 3301 | gtcatttcac tcaacagctc ggcccagctc tgaaacttgg gaaggtgagg gattcagggg |
| 3361 | aggtcagagg atcccacttc ctgagcatgg gccatcactg ccagtcaggg gctgggggct |
| 3421 | gagccctcac ccccccctcc cctactgttc tcatggtgtt ggcctcgtgt ttgctatgcc |
| 3481 | aactagtaga accttctttc ctaatcccct tatcttcatg gaaatggact gactttatgc |
| 3541 | ctatgaagtc cccaggagct acactgatac tgagaaaacc aggctctttg gggctagaca |
| 3601 | gactggcaga gagtgagatc tccctctctg agaggagcag cagatgctca cagaccacac |
| 3661 | tcagctcagg ccccttggag caggatggct cctctaagaa tctcacagga cctcttagtc |
| 3721 | tctgccctat acgccgcctt cactccacag cctcacccct cccaccccca tactggtact |
| 3781 | gctgtaatga gccaagtggc agctaaaagt tgggggtgtt ctgcccagtc ccgtcattct |
| 3841 | gggctagaag gcaggggacc ttggcatgtg gctggccaca ccaagcagga agcacaaact |
| 3901 | cccccaagct gactcatcct aactaacagt cacgccgtgg gatgtctctg tccacattaa |
| 3961 | actaacagca ttaatgca |

In some embodiments with respect to CSF-1 and/or CSF-1R, the inhibitor is a DNA inhibitor, an RNA inhibitor, or both. DNA inhibitors include quinolones that act upon DNA gyrase as a topoisomerase inhibitor, or it could be nitrofurantoin and/or metronidazole. RNA inhibitors may act upon DNA-dependent RNA polymerase, such as rifampin. Antifolates act as inhibitors of both RNA and DNA and may be used. In specific embodiments, the inhibitor inhibits DNA or RNA that encodes CSF-1R, such as by RNA interference.

### V. ANTIBODIES

In specific embodiments, an antibody that targets CSF-1 or CSF-1R is utilized to treat cancer, including MRD, and including for ctDNA+ individuals in at least some cases. Examples include AMB001 (AmMax Bio, Inc); FPA008 (Five Prime Therapeutics, Inc., Bristol-Meyers Squibb, and Ono Pharmaceutical Co. Ltd.); LY3022855 (Eli Lilly and Company); RG7155 (SynOx Therapeutics Limited, M.D. Anderson Cancer Center, and Hoffmann-La Roche); SNDX6532 (University of Utah Incyte Corporation and Syndax Pharma|AstraZeneca-Hopkins); AFS98 (Thermo Fisher Scientific), and/or M279 (Amgen). The antibody may be human, humanized, chimeric, or murine.

In some embodiments, AMB001 comprises a heavy chain comprising the amino acid sequence of SEQ ID NO:3 and a light chain comprising the amino acid sequence of SEQ ID NO: 4.

(SEQ ID NO: 3)
QVQLVQSGAEVKKPGASVKVSCKASGYTFTSYGISWVRQAPGQGLEWM
GWISAYNGNTNYAQKLQGRVTMTTDTSTSTAYMELRSLRSDDTAVYYC
ARESWFGEVFFDYWGQGTLVTVSSASTKGPSVFPLAPCSRSTSESTAA
LGCLVKDYFPEPVTVSWNSGALTSGVHTFPAVLQSSGLYSLSSVVTVP
SSNFGTQTYTCNVDHKPSNTKVDKTVERKCCVECPPCPAPPVAGPSVF
LFPPKPKDTLMISRTPEVTCVVVDVSHEDPEVQFNWYVDGVEVHNAKT
KPREEQFNSTERVVSVLTVVHQDWLNGKEYKCKVSNKGLPAPIEKTIS
KTKGQPREPQVYTLPPSREEMTKNQVSLTCLVKGFYPSDIAVEWESNG
QPENNYKTTPPMLDSDGSFFLYSKLTVDKSRWQQGNVFSCSVMHEALH
NHYTQKSLSLSPGK
(SEQ ID NO: 4)
DIVMTQSPDSLAVSLGERATINCKSSQSVLDSSDNKNYLAWYQQKPGQ
PPKLLIYWASNRESGVPDRFSGSGSGTDFTLTISSLQAEDVAVYYCQQ
YYSDPFTFGPGTKVDIKRTVAAPSVFIFPPSDEQLKSGTASVVCLLNN
FYPREAKVQWKVDNALQSGNSQESVTEQDSKDSTYSLSSTLTLSKADY
EKHKVYACEVTHQGLSSPVTKSFNRGEC

In particular embodiments, the antibody comprises the CDR1, CDR2, and CDR3 of SEQ ID NO:3 and/or SEQ ID NO:4.

In specific embodiments, the CDR1 of the VH chain may be SYGIS (SEQ ID NO: 5), GYTFTSYG (SEQ ID NO:6), or GYTFTSY (SEQ ID NO:7). In specific embodiments, the CDR2 of the VH chain may be WISAYNGNTNYAQKLQG (SEQ ID NO:8), ISAYNGNT (SEQ ID NO:9), or SAYNGN (SEQ ID NO:10). In specific embodiments, the CDR3 of the VH chain may be ESWFGEVFFDY (SEQ ID NO:11) or ARESWFGEVFFDY (SEQ ID NO: 12). In specific embodiments, the CDR1 of the VL chain may be KSSQSVLDSSDNKNYLA (SEQ ID NO:13) or QSVLDSSDNKNY (SEQ ID NO:14). In specific embodiments, the CDR2 of the VL chain may be WASNRES (SEQ ID NO:15) or WAS (SEQ ID NO:16). In specific embodiments, the CDR3 of the VL chain may be QQYYSDPFT (SEQ ID NO:17).

In certain embodiments, with respect to SEQ ID NO:3, any of the CDRs of the VH chain may be 1, 2, 3, or more amino acids shorter or longer on the N-terminal side of any of the sequences above and/or 1, 2, 3, or more amino acids shorter or longer on the C-terminal side of any of the sequences above. In certain embodiments, with respect to SEQ ID NO: 4, any of the CDRs of the VL chain may be 1, 2, 3, or more amino acids shorter or longer on the N-terminal side of any of the sequences above and/or 1, 2, 3, or more amino acids shorter or longer on the C-terminal side of any of the sequences above.

In specific embodiments, the inhibitor is in an aqueous pharmaceutical composition comprising at least 105 mg/mL of an antibody, a salt of arginine selected from the group consisting of arginine glutamate, arginine aspartate, and arginine HCl, histidine, and polysorbate (PS) selected from the group consisting of PS 80 and PS 20, at a pH of 5.0-6.0, wherein the antibody comprises a heavy chain comprising the amino acid sequence of SEQ ID NO: 37 and a light chain comprising the amino acid sequence of SEQ ID NO: 4.

In some embodiments, the antibody is a human or humanized antibody. In some embodiments, antibody is an anti-CSF1 or anti-CSF1R antibody. An example anti-CSF1R antibody is AMB001, which has a heavy chain variable region of SEQ ID NO:3 and a light chain variable region of SEQ ID NO: 4 which were prepared and tested in PCT application WO 2009/026303. The epitopes are mainly located at the N-terminus Ig-like loop 1 and Ig-like loop 2 of human CSF1R, and requires the presence of both the loop 1 and loop 2 regions.

Additional examples of anti-CSF1 and anti-CSF1R antibodies are provided in Tables 1A-1B. Their sequences are provided in Tables 2A-2B. In some embodiments, the antibody is emactuzumab, cabiralizumab, axatilimab, IMC-CS4, lacnotuzumab or PD-0360324.

TABLE 1A
Example Anti-CSF1R Antibodies

| Name | Other Names | Type |
|---|---|---|
| Emactuzumab | RG7155, or RO5509554 | IgG1 humanized |
| Cabiralizumab | FPA008 | IgG4 humanized |
| Axatilimab | SNDX-6352 | IgG4 humanized |
| IMC-CS4 | LY3022855 | IgG1 human |
| AM001 | | IgG2 human |

TABLE 1B
Example Anti-CSF1 Antibodies

| Name | Other Names | Type |
|---|---|---|
| Lacnotuzumab | MCS110 | IgG1 human |
| PD-0360324 | | IgG2 human |

TABLE 2A
Sequences of Example Anti-CSF1R Antibodies

| Antibody | Protein Sequences |
|---|---|
| Emactuzumab | Heavy chain (SEQ ID NO: 20) |
| (RG7155, or | QVQLVQSGAEVKKPGASVKVSCKASGYTFTSYDISWVRQAPGQGLEWMGVIWTDGGTNYA |
| RO5509554) | QKLQGRVTMTTDTSTSTAYMELRSLRSDDTAVYYCARDQRLYFDVWGQGTTVTVSSASTK |
| | GPSVFPLAPSSKSTSGGTAALGCLVKDYFPEPVTVSWNSGALTSGVHTFPAVLQSSGLYS |
| | LSSVVTVPSSSLGTQTYICNVNHKPSNTKVDKKVEPKSCDKTHTCPPCPAPELLGGPSVF |
| | LFPPKPKDTLMISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLPPSRDELTKN |
| | QVSLTCLVKGFYPSDIAVEWESNGQPENNYKTTPPVLDSDGSFFLYSKLTVDKSRWQQGN |
| | VFSCSVMHEALHNHYTQKSLSLSPGK |
| | Light chain (SEQ ID NO: 21) |
| | DIQMTQSPSSLSASVGDRVTITCRASEDVNTYVSWYQQKPGKAPKLLIYAASNRYTGVPS |
| | RFSGSGSGTDFTLTISSLQPEDFATYYCQQSFSYPTFGQGTKLEIKRTVAAPSVFIFPPS |
| | DEQLKSGTASVVCLLNNFYPREAKVQWKVDNALQSGNSQESVTEQDSKDSTYSLSSTLTL |
| | SKADYEKHKVYACEVTHQGLSSPVTKSFNRGEC |
| Cabiralizumab | Heavy chain (SEQ ID NO: 22) |
| (FPA008) | QVQLVQSGAEVKKPGSSVKVSCKASGYTFTDNYMIWVRQAPGQGLEWMGDINPYNGGTTF |
| | NQKFKGRVTITADKSTSTAYMELSSLRSEDTAVYYCARESPYFSNLYVMDYWGQGTLVTV |
| | SSASTKGPSVFPLAPCSRSTSESTAALGCLVKDYFPEPVTVSWNSGALTSGVHTFPAVLQ |
| | SSGLYSLSSVVTVPSSSLGTKTYTCNVDHKPSNTKVDKRVESKYGPPCPPCPAPEFLGGP |
| | SVFLFPPKPKDTLMISRTPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSRLTVDKSRWQ |
| | EGNVFSCSVMHEALHNHYTQKSLSLSLGK |
| | Light chain (SEQ ID NO: 23) |
| | EIVLTQSPATLSLSPGERATLSCKASQSVDYDGDNYMNWYQQKPGQAPRLLIYAASNLES |
| | GIPARFSGSGSGTDFTLTISSLEPEDFAVYYCHLSNEDLSTFGGGTKVEIKRTVAAPSVF |
| | IFPPSDEQLKSGTASVVCLLNNFYPREAKVQWKVDNALQSGNSQESVTEQDSKDSTYSLS |
| | STLTLSKADYEKHKVYACEVTHQGLSSPVTKSFNRGEC |
| IMC-CS4 | Heavy chain (SEQ ID NO: 24) |
| (LY3022855) | QDQLVESGGGVVQPGRSLRLSCAASGFTFSSYGMHWVRQAPGEGLEWVAVIWYDGSNKYY |
| | ADSVKGRFTISRDNSKNTLYLQMNSLRAEDTAVYYCARGDYEVDYGMDVWGQGTTVTVAS |
| | ASTKGPSVFPLAPSSKSTSGGTAALGCLVKDYFPEPVTVSWNSGALTSGVHTFPAVLQSS |
| | GLYSLSSVVTVPSSSLGTQTYICNVNHKPSNTKVDKRVEPKSCDKTHTCPPCPAPELLGG |
| | PSVFLFPPKPKDTLMISRTPEVTCVVVDVSHEDPEVKFNWYVDGVEVHNAKTKPREEQYN |
| | STYRVVSVLTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSKLTVDKSRW |
| | QQGNVFSCSVMHEALHNHYTQKSLSLSPGK |
| | Light chain (SEQ ID NO: 25) |
| | AIQLTQSPSSLSASVGDRVTITCRASQGISNALAWYQQKPGKAPKLLIYDASSLESGVPS |
| | RFSGSGSGTDFTLTISSLQPEDFATYYCQQFNSYPWTFGQGTKVEIKRTVAAPSVFIFPP |
| | SDEQLKSGTASVVCLLNNFYPREAKVQWKVDNALQSGNSQESVTEQDSKDSTYSLSSTLT |
| | LSKADYEKHKVYACEVTHQGLSSPVTKSFNRGEC |
| Axatilimab | Heavy chain (SEQ ID NO: 26) |
| (SNDX-6352) | EVTLKESGPALVKPTQTLTLTCTFSGFSLTTYGMGVGWIRQPPGKALEWLANIWWDDDKY |
| | YNPSLKNRLTISKDTSKNQVVLTMTNMDPVDTATYYCARIGPIKYPTAPYRYFDFWGQGT |
| | MVTVSSASTKGPSVFPLAPCSRSTSESTAALGCLVKDYFPEPVTVSWNSGALTSGVHTFP |
| | AVLQSSGLYSLSSVVTVPSSSLGTKTYTCNVDHKPSNTKVDKRVESKYGPPCPPCPAPEF |
| | LGGPSVFLFPPKPKDTLMISRTPEVTCVVVDVSQEDPEVQFNWYVDGVEVHNAKTKPREE |
| | QFNSTYRVVSVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFFLYSRLTVDK |
| | SRWQEGNVFSCSVMHEALHNHYTQKSLSLSLGK |
| | Light chain (SEQ ID NO: 27) |
| | DIQMTQSPSSLSASVGDRVTITCLASEDIYDNLAWYQQKPGKAPKLLIYYASSLQDGVPS |
| | RFSGSGSGTDYTLTISSLQPEDFATYYCLQDSEYPWTFGGGTKVEIKRTVAAPSVFIFPP |
| | SDEQLKSGTASVVCLLNNFYPREAKVQWKVDNALQSGNSQESVTEQDSKDSTYSLSSTLT |
| | LSKADYEKHKVYACEVTHQGLSSPVTKSFNRGEC |

TABLE 2B
Sequences of Example Anti-CSF1 Antibodies

| Antibody | Protein Sequences |
|---|---|
| Lacnotuzumab | Heavy chain (SEQ ID NO: 28) |
| (MCS110) | QVQLQESGPGLVKPSQTLSLTCTVSDYSITSDYAWNWIRQFPGKGLEWMGYISYSGSTSY |
| | NPSLKSRITISRDTSKNQFSLQLNSVTAADTAVYYCASFDYAHAMDYWGQGTTVTVSSAS |
| | TKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKRVEPKSCDKTHTCPPCPAPELLGGPS |
| | VFLFPPKPKDTLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYTLPPSREEMT |
| | KNQVSLTCLVKGFYPSDIAVEWESNGQPENNYKTTPPVLDSDGSFFLYSKLTVDKSRWQQ |
| | GNVFSCSVMHEALHNHYTQKSLSLSPGK |
| | Light chain (SEQ ID NO: 29) |
| | DIVLTQSPAFLSVTPGEKVTFTCQASQSIGTSIHWYQQKTDQAPKLLIKYASESISGIPS |
| | RFSGSGSGTDFTLTISSVEAEDAADYYCQQINSWPTTFGGGTKLEIKRTVAAPSVFIFPP |
| | SDEQLKSGTASVVCLLNNFYPREAKVQWKVDNALQSGNSQESVTEQDSKDSTYSLSSTLT |
| | LSKADYEKHKVYACEVTHQGLSSPVTKSFNRGEC |
| MCS110 var | Heavy chain (SEQ ID NO: 30) |
| | DVQLQESGPGLVKPSQSLSLTCTVTDYSITSDYAWNWIRQFPGNKLEWMGYISYSGSTSY |
| | NPSLKSRISITRDTSKNQFFLQLNSVTTEDTATYYCASFDYAHAMDYWGQGTSVTVSSAK |
| | TTAPSVYPLAPVCGDTTGSSVTLGCLVKGYFPEPVTLTWNSGSLSSGVHTFPAVLQSDLY |
| | TLSSSVTVTSSTWPSQSITCNVAHPASSTKVDKKIEPRGPTIKPCPPCKCPAPNLLGGPS |
| | VFIFPPKIKDVLMISLSPIVTCVVVDVSEDDPDVQISWFVNNVEVHTAQTQTHREDYNST |
| | LRVVSALPIQHQDWMSGKEFKCKVNNKDLPAPIERTISKPKGSVRAPQVYVLPPPEEEMT |
| | KKQVTLTCMVTDFMPEDIYVEWTNNGKTELNYKNTEPVLDSDGSYFMYSKLRVEKKNWVE |
| | RNSYSCSVVHEGLHNHHTTKSFSRTPG |
| | Light chain (SEQ ID NO: 31) |
| | DILLTQSPAILSVSPGERVSFSCRASQSIGTSIHWYQQRTNGSPRLLIKYASESISGIPS |
| | RFSGSGSGTDFTLSINSVESEDIADYYCQQINSWPTTFGGGTKLEIKRADAAPTVSIFPP |
| | SSEQLTSGGASVVCFLNNFYPKDINVKWKIDGSERQNGVLNSWTDQDSKDSTYSMSSTLT |
| | LTKDEYERHNSYTCEATHKTSTSPIVKSFNRNEC |
| PD-0360324 | Heavy chain (SEQ ID NO: 32) |
| | EVQLVESGGGLVQPGGSLRLSCAASGFTFSSFSMTWVRQAPGKGLEWVSYISSRSSTISY |
| | ADSVKGRFTISRDNAKNSLYLQMNSLRDEDTAVYYCARDPLLAGATFFDYWGQGTLVTVS |
| | SASTKGPSVFPLAPCSRSTSESTAALGCLVKDYFPEPVTVSWNSGALTSGVHTFPAVLQS |
| | SGLYSLSSVVTVPSSNFGTQTYTCNVDHKPSNTKVDKTVERKCCVECPPCPAPPVAGPSV |
| | FLFPPKPKDTLMISRTPEVTCVVVDVSHEDPEVQFNWYVDGVEVHNAKTKPREEQFNSTF |
| | RVVSVLTVVHQDWLNGKEYKCKVSNKGLPAPIEKTISKTKGQPREPQVYTLPPSREEMTK |
| | NQVSLTCLVKGFYPSDIAVEWESNGQPENNYKTTPPMLDSDGSFFLYSKLTVDKSRWQQG |
| | NVFSCSVMHEALHNHYTQKSLSLSPGK |
| | Light chain (SEQ ID NO: 33) |
| | EFVLTQSPGTLSLSPGERATLSCRASQSVSSSYLAWYQQKPGQAPRLLIYGASSRATGIP |
| | DRFSGSGSGTDFTLTISRLEPEDFAVYYCQQYGSSPLTFGGGTKVEIKRTVAAPSVFIFP |
| | PSDEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC |

Emactuzumab (also known as RG7155 and RO5509554) is a clinical stage humanized IgG1 CSF1R targeted antibody designed to target and deplete macrophages in the tumor tissue. It has shown a favorable safety profile in patients and encouraging efficacy for TGCT. Emactuzumab is under investigation in clinical trial NCT01494688—“A Study of RO5509554 as Monotherapy and in Combination with Paclitaxel in Participants With Advanced Solid Tumors.”

Cabiralizumab (also known as FPA008) is under investigation in clinical trial NCT03502330—“APX005M With Nivolumab and Cabiralizumab in Advanced Melanoma, Non-small Cell Lung Cancer or Renal Cell Carcinoma.” Cabiralizumab is a humanized IgG4 anti-CSF1R monoclonal antibody with a single amino acid substitution in the hinge region to prevent hemi-dimer exchange.

IMC-CS4 (also known as LY3022855) is a human IgG1 antibody (mAb) targeting CSF1R. IMC-CS4 is under investigation in clinical trial NCT01346358—“A Study of IMC-CS4 in Subjects With Advanced Solid Tumors.”

Axatilimab (also known as SNDX-6352) is a humanized, full-length IgG4 antibody with high affinity to CSF-1R. Axatilimab affects the migration, proliferation, differentiation, and survival of monocytes and macrophages by binding to CSF-1R and blocking its activation by its two known ligands, CSF-1 and IL-34. Axatilimab is currently being evaluated in a Phase ½ clinical trial in patients with cGVHD.

Lacnotuzumab (also known as MCS110) is a high-affinity human engineered IgG1 anti-CSF1 antibody that blocks the ability of CSF1R to drive proliferation in responsive cells. Lacnotuzumab is under investigation in clinical trial NCT01643850-“MCS110 in Patients With Pigmented Villonodular Synovitis (PVNS).”

PD-0360324 is a fully human immunoglobulin G2 monoclonal antibody against CSF1 investigated for treating cutaneous lupus erythematosus (CLE). It is also being tested for its combination with Cyclophosphamide in treating patients with recurrent high-grade epithelial ovarian, primary peritoneal, or fallopian tube cancer.

In particular embodiments, an antibody encompasses CDR1, CDR2, and/or CDR3 of any one or more of SEQ ID NOs: 20-33.

Aspects of the disclosure relate to CSF-1 or CSF-1R antibodies comprising a heavy or light chain, or fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: X-ray crystallography, nuclear magnetic resonance spectroscopy, site-directed mutagenesis mapping, protein display arrays, sec, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Scla-Culang et al., Front Immunol. 2013; 4:302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ϵ) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24:8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### A. Functional Antibody Fragments and Antigen-Binding Fragments

Certain aspects relate to antibody fragments, such as antibody fragments that bind to and modulate activity. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### B. Fragment Crystallizable Region, Fc

A fragment crystallizable region (Fc region) contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### VI. OBTAINING ENCODED POLYPEPTIDE EMBODIMENTS

In some aspects, there are nucleic acid molecule encoding polypeptides (e.g., antibodies, antibody fragments, CARs, and bispecific molecules). These may be generated by methods known in the art, e.g., isolated from B cells of mice that have been immunized and isolated, phage display, expressed in any suitable recombinant expression system and allowed to assemble to form antibody molecules.

### A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

### 1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the antibodies, or antigen-binding fragments thereof, DNAs encoding partial or full-length light and heavy chains are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote-and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### 3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by acoustofluidic gene delivery (see for example, WO2018148715 and Belling et al., PNAS 117 (20), 10976-10982, each of which are incorporated by reference for all purposes); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. No. 5,610,042; 5,322,783, 5,563,055, 5,550,318, 5,538,877 and 5,538,880, and each incorporated herein by reference); by biophysical methods such as mechanical cell squeezing (Sharei et al., PNAS 2013-110 (6) 2082-2087; PNAS 2018-115 E10907 and WO2017041050, both of which are incorporated by reference for all purposes), by agitation with silicon carbide fibers (Kaeppler et al., 1990; U.S. Pat. No. 5,302,523 and 5,464,765, each incorporated herein by reference); by Agrobacterium mediated transformation (U.S. Pat. No. 5,591,616 and 5,563,055, each incorporated herein by reference); or by PEG mediated transformation of protoplasts (Omirullch et al., 1993; U.S. Pat. No. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 4. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### VII. ADDITIONAL THERAPIES

### A. Immunostimulators

In some embodiments, the method further comprises administration of an additional therapy or agent. In some embodiments, the additional therapy is an immunostimulator. The term “immunostimulator” as used herein refers to a compound that can stimulate an immune response in a subject, and may include an adjuvant. In some embodiments, an immunostimulator is an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

In some embodiments, the additional therapy comprises an agonist for pattern recognition receptors (PRR), including, but not limited to Toll-Like Receptors (TLRs), specifically TLRs 2, 3, 4, 5, 7, 8, 9 and/or combinations thereof. In some embodiments, additional therapies comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. In some embodiments, the additional therapies also may comprise immunostimulatory RNA molecules, such as but not limited to dsRNA, poly I: C or poly I: poly C12U (available as Ampligen®, both poly I: C and poly I: polyC12U being known as TLR3 stimulants), and/or those disclosed in F. Heil et al., “Species-Specific Recognition of Single-Stranded RNA via Toll-like Receptor 7 and 8” Science 303 (5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann ct al., “Modified oligoribonucleotide analogs with enhanced immunostimulatory activity” U.S. Pat. Appl. Publ. U.S. Pat. No. 20,062,41076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an additional therapy may be a TLR-4 agonist, such as bacterial lipopolysaccharide (LPS), VSV-G, and/or HMGB-1. In some embodiments, additional therapies may comprise TLR-5 agonists, such as flagellin, or portions or derivatives thereof, including but not limited to those disclosed in U.S. Pat. Nos. 6,130,082, 6,585,980, and 7,192,725.

In some embodiments, additional therapies may be proinflammatory stimuli released from necrotic cells (e.g., urate crystals). In some embodiments, additional therapies may be activated components of the complement cascade (e.g., CD21, CD35, etc.). In some embodiments, additional therapies may be activated components of immune complexes. Additional therapies also include complement receptor agonists, such as a molecule that binds to CD21 or CD35. In some embodiments, the complement receptor agonist induces endogenous complement opsonization of the synthetic nanocarrier. In some embodiments, immunostimulators are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer.

### B. Immunotherapies

In some embodiments, the additional therapy comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

### 1. Inhibition of Co-Stimulatory Molecules

In some embodiments, the immunotherapy comprises an inhibitor of a co-stimulatory molecule. In some embodiments, the inhibitor comprises an inhibitor of B7-1(CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (i.e. a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 3. CAR Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, although in some cases the cell is any immune effector cell, including NK cells, NKT cells, macrophages, dendritic cells, B cells, and so forth. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

### 4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 5. Adoptive T-Cell Therapy

Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### 6. Checkpoint Inhibitors and Combination Treatment

In some embodiments, the additional immunotherapy comprises immune checkpoint inhibitors. Certain embodiments are further described below.

PD-1 can act in the tumor microenvironment where T cells encounter an infection or tumor. Activated T cells upregulate PD-1 and continue to express it in the peripheral tissues. Cytokines such as IFN-gamma induce the expression of PDLI on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners arc PDL1 and/or PDL2. In another embodiment, a PDLI inhibitor is a molecule that inhibits the binding of PDLI to its binding partners. In a specific aspect, PDLI binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85,90, 95, 97, or 99% (or any derivable range therein) variable region amino acid sequence identity with the above-mentioned antibodies.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (sec, e.g., WO01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### C. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

### D. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### E. Neoantigens

In some embodiments, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### F. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 weck intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### G. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### H. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### VIII. CHIMERIC ANTIGEN RECEPTORS (CARS) AND METHODS OF GENERATING CARS

In certain embodiments, one or more types of immune effector cells expressing a CAR comprising a component (such as an scFv) that targets CSF-1 or CSF-1R is utilized. The immune effector cells may be T cells, natural killer (NK) cells, NK T cells, dendritic cells, B cells, macrophages, etc. The term “chimeric antigen receptor” or “CAR” refers to engineered receptors, which graft an arbitrary specificity onto an immune effector cell. These receptors are used to graft the specificity of a monoclonal antibody onto an immune cell; with transfer of their coding sequence facilitated by retroviral or lentiviral vectors. The receptors are called chimeric because they are composed of parts from different sources.

The most common form of these molecules are fusions of single-chain variable fragments (scFv) derived from monoclonal antibodies, fused to CD3-zeta transmembrane and endodomain, CD28 or 41BB intracellular domains, or combinations thereof. Such molecules result in the transmission of a signal in response to recognition by the scFv of its target. When immune cells express this molecule (as an example achieved by oncoretroviral vector transduction), they recognize and kill target cells that express the target.

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows the scFv to orient in different directions to enable antigen binding. The transmembrane domain is a typical hydrophobic alpha helix usually derived from the original molecule of the signaling endodomain which protrudes into the cell and transmits the desired signal.

The CARs may comprise at least one extracellular and at least one intracellular domain. An extracellular domain can comprise a target-specific binding element otherwise referred to as an antigen-or ligand-binding moiety that specifically binds to any particular antigen of interest.

The intracellular domain or the cytoplasmic domain may comprise, one or more costimulatory signaling region(s), and/or a zeta chain portion. The costimulatory signaling region refers to a portion of the CAR comprising the intracellular domain of a costimulatory molecule. Costimulatory molecules may be cell surface molecules other than antigen receptors or their ligands that are required for an efficient response of immune cells to antigen.

### A. Signal Peptide

Polypeptides of the present disclosure may comprise a signal peptide. A “signal peptide” refers to a peptide sequence that directs the transport and localization of the protein within a cell, e.g., to a certain cell organelle (such as the endoplasmic reticulum) and/or the cell surface. A signal peptide may direct the nascent protein into the endoplasmic reticulum. This is essential if a receptor is to be glycosylated and anchored in the cell membrane. Generally, the signal peptide natively attached to the amino-terminal most component is used (e.g. in an scFv with orientation light chain-linker-heavy chain, the native signal of the light-chain is used).

The signal peptide may be cleaved after passage of the endoplasmic reticulum (ER), i.e., is a cleavable signal peptide. A restriction site may be at the carboxy end of the signal peptide to facilitate cleavage.

### B. Antigen Binding Domain

Polypeptides of the present disclosure may comprise one or more antigen binding domains. An “antigen binding domain” describes a region of a polypeptide capable of binding to an antigen under appropriate conditions. An antigen binding domain may be a single-chain variable fragment (scFv) based on one or more antibodies (e.g., CD20 antibodies). An antigen binding domain may comprise a variable heavy (VH) region and a variable light (VL) region, with the VH and VL regions being on the same polypeptide. The antigen binding domain may comprise a linker between the VH and VL regions. A linker may enable the antigen binding domain to form a desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10-5M, 10-6M, 10-7M, 10-8M, 10-9M, 10-10M, 10-11M, 10-12M, or 10-13M. The KD of the antigen binding region, such as the variable regions (heavy chain and/or light chain variable region), or of the CDRs may be at least 10-5M, 10-6M, 10-7M, 10-8M, 10-9M, 10-10M, 10-11M, 10-12M, or 10-13M (or any derivable range therein).

Binding affinity, KA, or KD can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

The polypeptide comprising the humanized binding region may have equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity and/or expression level in host cells, compared to a polypeptide comprising a non-humanized binding region, such as a binding region from a mouse.

The framework regions, such as FR1, FR2, FR3, and/or FR4 of a huma FRamework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

The framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a huma FRamework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

### C. Extracellular Peptide Spacer

Between the extracellular domain and the transmembrane domain of the CAR, and/or between the cytoplasmic domain and the transmembrane domain of the CAR, there may be incorporated a spacer domain. As used herein, the term “spacer domain” generally means any oligo-or polypeptide that functions to link the transmembrane domain to, either the extracellular domain or, the cytoplasmic domain in the polypeptide chain. An extracellular spacer may link the antigen-binding domain to the transmembrane domain. A peptide spacer may be flexible enough to allow the antigen-binding domain to orient in different directions to facilitate antigen binding.

The spacer may comprise the hinge region from IgG. The spacer may comprise or further comprise the CH2CH3 region of immunoglobulin and portions of CD3. The CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. The spacer may be from IgG4. An extracellular spacer may comprise a hinge region.

As used herein, the term “hinge” refers to a flexible polypeptide connector region (also referred to herein as “hinge region” or “spacer”) providing structural flexibility and spacing to flanking polypeptide regions and can consist of natural or synthetic polypeptides. A “hinge” or “spacer” derived from an immunoglobulin (e.g., IgG1) is generally defined as stretching from Glu216 to Pro230 of human IgG1, for example (Burton (1985) Molec. Immunol., 22:161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425. The hinge region can include a complete hinge region derived from an antibody of a different class or subclass from that of the CH1 domain. The term “hinge” can also include regions derived from CD8 and other receptors that provide a similar function in providing flexibility and spacing to flanking regions. Other alternatives include the CH2CH3 region of immunoglobulin and portions of CD3.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). The extracellular spacer may consist of or comprise a hinge region from an immunoglobulin (e.g. IgG). Immunoglobulin hinge region amino acid sequences are known in the art; see, e.g., Tan et al. (1990) Proc. Natl. Acad. Sci. USA 87:162; and Huck et al. (1986) Nucl. Acids Res.

The length of an extracellular spacer may have effects on the CAR's signaling activity and/or the CAR-T cells' expansion properties in response to antigen-stimulated CAR signaling. A shorter spacer such as less than 50, 45, 40, 30, 35, 30, 25, 20, 15, 14, 13, 12, 11, or 10 amino acids is used. A longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

As non-limiting examples, an immunoglobulin hinge region can include one of the following amino acid sequences:

The extracellular spacer can comprise an amino acid sequence of a human IgG1, IgG2, IgG3, or IgG4 hinge region. The extracellular spacer may also include one or more amino acid substitutions and/or insertions and/or deletions compared to a wild-type (naturally-occurring) hinge region.

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. The extracellular spacer may consist essentially of a hinge, CH2, and/or CH3 region, meaning that the hinge, CH2, and/or CH3 region is the only identifiable region present and all other domains or regions are excluded, but further amino acids not part of an identifiable region may be present.

### D. Transmembrane Domain

With respect to the transmembrane domain, the CAR can be designed to comprise a transmembrane domain that is fused to the extracellular domain of the CAR. A transmembrane domain may be a hydrophobic alpha helix that spans the membrane. Different transmembrane domains may result in different receptor stability.

The transmembrane domain that naturally is associated with one of the domains in the CAR may be used. In some instances, the transmembrane domain can be selected or modified by amino acid substitution to avoid binding of such domains to the transmembrane domains of the same or different surface membrane proteins to minimize interactions with other members of the receptor complex.

The transmembrane domain can be derived either from a natural or from a synthetic source. Where the source is natural, the domain may be derived from any membrane-bound or transmembrane protein. Illustrative, but non-limiting, examples of transmembrane regions of particular use in the CAR constructs contemplated here can be derived from (i.e. comprise at least the transmembrane region(s) of) the alpha, beta or zeta chain of the T-cell receptor, CD28, CD3 epsilon, CD45, CD4, CD5, CD8, CD9, CD 16, CD22, CD33, CD37, CD64, CD80, CD86,CD134, CD137, CD154. Alternatively, the transmembrane domain can be synthetic, in which case it can comprise predominantly hydrophobic residues such as leucine and valine. A triplet of phenylalanine, tryptophan and valine may be be found at each end of a synthetic transmembrane domain. Optionally, a short oligo-or polypeptide linker, e.g., between 2 and about 10 amino acids in length may form the linkage between the transmembrane domain and the cytoplasmic signaling domain of the CAR. A glycine-serine doublet may provide a particularly suitable linker.

The transmembrane domain is interposed between the extracellular spacer and the cytoplasmic region. The transmembrane domain may be interposed between the extracellular spacer and one or more costimulatory regions. A linker may be between the transmembrane domain and the one or more costimulatory regions.

Any transmembrane domain that provides for insertion of a polypeptide into the cell membrane of a eukaryotic (e.g., mammalian) cell may be suitable for use. The transmembrane domain may be derived from CD28, CD8, CD4, CD3-zeta, CD134, or CD7.

Exemplary transmembrane domains useful in any of the aspects of the disclosure include those in the table below:

### E. Cytoplasmic Domain

The cytoplasmic domain or otherwise the intracellular signaling domain of the CAR is responsible for activation of at least one of the normal effector functions of the immune cell in which the CAR has been placed. After antigen and/or ligand recognition, receptors cluster and a signal is transmitted to the cell through the cytoplasmic region. The cytoplasmic region may comprise an intracellular signaling domain. An intracellular signaling domain may comprise a primary signaling domain and one or more costimulatory domains. The costimulatory domains described herein may be part of the cytoplasmic region.

The term “effector function” refers to a specialized function of a cell. An effector function of a T cell, for example, may be cytolytic activity, or helper activity including the secretion of cytokines. Thus the term “intracellular signaling domain” refers to the portion of a protein that transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the intracellular signaling domain sufficient to transduce the effector function signal.

Cytoplasmic regions and/or costimulatory regions suitable for use in the CARs of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. The cytoplasmic region may comprise at least one (e.g., one, two, three, four, five, six, etc.) ITAM motif as described herein. The cytoplasmic region may comprise DAP10/CD28 type signaling chains. The cytoplasmic region may comprise CD3-zeta, DAP10, CD28, 2B4, DNAM-1, 4-1BB, OX40, CD30, CD40, ICOS, lymphocyte function-associated antigen-1 (LFA-1), CD2, CD7, LIGHT, and NKG2C type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2 (L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2 (L/I))(X3)n(YX1X2(L/I)), where n is an integer from 6 to 8, and each of the 6-8 X3 can be any amino acid.

A suitable cytoplasmic region may be an ITAM motif-containing a portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCERIG (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Exemplary cytoplasmic regions are known in the art. The cytoplasmic regions shown below also provide examples of regions that may be incorporated in a CAR of the disclosure:

The cytoplasmic region may be derived from DAP12 (also known as TYROBP; TYRO protein tyrosine kinase binding protein; KARAP; PLOSL; DN AX-activation protein 12; KAR-associated protein; TYRO protein tyrosine kinase-binding protein; killer activating receptor associated protein; killer-activating receptor-associated protein; etc.). A suitable cytoplasmic region can comprise an ITAM motif-containing a portion of the full length DAP12 amino acid sequence.

The cytoplasmic region may be derived from FCERIG (also known as FCRG; Fc epsilon receptor I gamma chain; Fc receptor gamma-chain; fc-epsilon R1-gamma; fcRgamma; fceRI gamma; high affinity immunoglobulin epsilon receptor subunit gamma; immunoglobulin E receptor, high affinity, gamma chain; etc.). A suitable cytoplasmic region may comprise an ITAM motif-containing a portion of the full length FCERIG amino acid sequence.

The cytoplasmic region may be derived from T cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T cell receptor T3 delta chain; T cell surface glycoprotein CD3 delta chain; etc.). A suitable cytoplasmic region can comprise an ITAM motif-containing a portion of the full length CD3 delta amino acid sequence.

The cytoplasmic region may be derived from T cell surface glycoprotein CD3 epsilon chain (also known as CD3e, T cell surface antigen T3/Leu-4 epsilon chain, T cell surface glycoprotein CD3 epsilon chain, AI504783, CD3, CD3epsilon, T3c, etc.). A suitable cytoplasmic region can comprise an ITAM motif-containing a portion of the full length CD3 epsilon amino acid sequence.

The cytoplasmic region may be derived from T cell surface glycoprotein CD3 gamma chain (also known as CD3G, T cell receptor T3 gamma chain, CD3-GAMMA, T3G, gamma polypeptide (TiT3 complex), etc.). A suitable cytoplasmic region can comprise an ITAM motif-containing a portion of the full length CD3 gamma amino acid sequence.

The cytoplasmic region may be derived from T cell surface glycoprotein CD3 zeta chain (also known as CD3Z, T cell receptor T3 zeta chain, CD247, CD3-ZETA, CD3H, CD3Q, T3Z, TCRZ, etc.). A suitable cytoplasmic region can comprise an ITAM motif-containing a portion of the full length CD3 zeta amino acid sequence.

The cytoplasmic region may be derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). A suitable cytoplasmic region can comprise an ITAM motif-containing a portion of the full length CD79A amino acid sequence.

Suitable cytoplasmic regions can comprise a CD28 type signaling chain. Further cytoplasmic regions suitable for use in the CARs of the disclosure include a ZAP70 polypeptide.

### F. Co-Stimulatory Region

The term “co-stimulatory ligand,” as the term is used herein, includes a molecule on an antigen presenting cell (e.g., an APC, dendritic cell, B cell, and the like) that specifically binds a cognate co-stimulatory molecule or domain on an immune effector cell, thereby providing a signal which, in addition to the primary signal to mediate the immune effector cell response, including, but not limited to, proliferation, activation, differentiation, and the like. A co-stimulatory ligand also encompasses, inter alia, an antibody that specifically binds with a co-stimulatory molecule present on an immune effector cell. A “co-stimulatory molecule” refers to the cognate binding partner on an immune effector cell that specifically binds with a co-stimulatory ligand, thereby mediating a co-stimulatory response by the immune effector, such as, but not limited to, proliferation and/or activation. A “co-stimulatory signal”, as used herein, refers to a signal that in combination with a primary signal, leads to immune cell activation, proliferation, and/or upregulation or downregulation of key molecules.

By the term “stimulation,” it is meant a primary response induced by binding of a stimulatory molecule with its cognate ligand, thereby mediating a signal transduction event, such as, but not limited to, signal transduction. Stimulation can mediate altered expression of certain molecules. A “stimulatory molecule,” as the term is used herein, means a molecule on an immune effector cell that specifically binds with a cognate stimulatory ligand present on an antigen presenting cell. A “stimulatory ligand,” as used herein, means a ligand that when present on an antigen presenting cell (e.g., an APC, a dendritic cell, a B-cell, and the like) can specifically bind with a cognate binding partner (referred to herein as a “stimulatory molecule”) on an immune effector cell, thereby mediating a primary response by the immune effector cell, including, but not limited to, activation, initiation of an immune response, proliferation, and the like.

Non-limiting examples of suitable costimulatory regions, such as those included in the cytoplasmic region, include, but are not limited to, polypeptides from 4-1BB (CD137), CD28, ICOS, OX-40, BTLA, CD27, CD30, CD40, GITR, 2B4, DNAM-1, lymphocyte function-associated antigen-1 (LFA-1), CD2, CD7, LIGHT, NKG2C, and HVEM.

A co-stimulatory region may have a length of at least, at most, or exactly 10, 15, 20, 25, 30, 35, 40, 50, 60, 70, 80, 90, 100, 150, 200, or 300 amino acids or any range derivable therein.

The costimulatory region may be derived from DAP10 (also known as HCST, DAP10, KAP10, PIK3AP, hematopoietic cell signal transducer; etc.). The costimulatory region may be derived from an intracellular portion of the transmembrane protein 4-1BB (also known as Tumor necrosis factor receptor superfamily member 9, TNFRSF9; CD137; CDw137; ILA; etc.). The costimulatory region may be derived from an intracellular portion of the transmembrane protein CD28 (also known as Tp44). The costimulatory region may be derived from an intracellular portion of the transmembrane protein ICOS (also known as inducible T-cell costimulatory, AILIM, CD278, and CVID1). The costimulatory region may be derived from an intracellular portion of the transmembrane protein OX-40 (also known as tumor necrosis factor receptor superfamily member 4, TNFRSF4, RP5-902P8.3, ACT35, CD134, OX40, TXGPIL). The costimulatory region may be derived from an intracellular portion of the transmembrane protein BTLA (also known as B-and T-Lymphocyte-Associated Protein, BTLA1 and CD272). The costimulatory region may be derived from an intracellular portion of the transmembrane protein CD27 (also known as S152, T14, Tumor Necrosis Factor Receptor Superfamily Member 7, TNFRSF7, and Tp55). The costimulatory region may be derived from an intracellular portion of the transmembrane protein CD30 (also known as tumor necrosis factor receptor superfamily member 8, TNFRSF8, DIS166E, and Ki-1). The costimulatory region may be derived from an intracellular portion of the transmembrane protein GITR (also known as tumor necrosis factor receptor superfamily member 18, TNFRSF18, RP5-902P8.2, AITR, CD357, ENERGEN, and GITR-D). The costimulatory region may be derived from an intracellular portion of the transmembrane protein HVEM (also known as tumor necrosis factor receptor superfamily member 14, TNFRSF14, RP3-395M20.6, ATAR, CD270, HVEA, LIGHTR, and TR2). The costimulatory region may be derived from 2B4 (also known as CD244, NAIL, NKR2B4, Nmrk, SLAMF4, CD244 molecule, etc.). The costimulatory region may be derived from DNAM-1 (also known as CD226, DNAMI, PTAI, TLiSAI, CD226 molecule, etc.). The costimulatory region may be derived from CD40 (also known as Bp50, CDW40, TNFRSF5, p50, CD40 (protein), CD40 molecule, etc.). The costimulatory region may be derived from LFA-1 (also known as lymphocyte function-associated antigen 1, integrin alpha L, ITGAL, CD11A, LFAIA, integrin subunit alpha L, etc.). The costimulatory region may be derived from CD2 (also known as Lymphocyte-Function Antigen-2, LFA-2, SRBC, T11, CD2 molecule, etc.). The costimulatory region may be derived from CD7 (also known as GP40, LEU-9, TP41, Tp40, CD7 molecule, etc.). The costimulatory region may be derived from LIGHT (also known as TNFSF14, CD258, HVEML, LIGHT, LTg, TR2, TNLGID, tumor necrosis factor superfamily member 14, etc.). The costimulatory region may be derived from NKG2C (also known as KLRC2, CD159c, NKG2-C, killer cell lectin like receptor C2, etc.).

Specific exemplary co-stimulatory domains are represented by the amino acid sequences below:

### G. Detection Peptides

The CARs described herein may further comprise a detection peptide or molecule. Suitable detection peptides include hemagglutinin; FLAG; c-myc, and the like. Other suitable detection peptides are known in the art.

### H. Peptide Linkers

The polypeptides of the disclosure may include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N-or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. The linker may comprise a repeat, such as a contiguous repeat of at least, at most, or exactly 2, 3, 4, 5, 6, 7, 8, 9, or 10 times, or any range derivable therein.

### IX. ADMINISTRATION OF THERAPEUTIC COMPOSITIONS

The therapy provided herein may comprise administration of at least one cancer therapy. The therapy may be administered in any suitable manner known in the art. Embodiments of the disclosure relate to compositions and methods comprising therapeutic compositions.

In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

The cancers amenable for treatment can include or exclude tumors of all types, locations, sizes, and characteristics. The MRD amenable for treatment can include or exclude tumors of all types, locations, sizes, and characteristics. The cancer may or may not comprise a solid tumor. The methods may include reducing tumor volume or treating cancers that are recurrent and/or metastatic. The cancer can comprise or exclude pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, lymphoid cancer, bronchial adenomas/carcinoids, tracheal cancer, lung cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutancous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myclodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pincal astrocytoma, pineal germinoma, pincoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

In some embodiments, the cancer (including MRD) being treated is a cancer that is difficult to treat, such as liver, pancreas, ovarian, brain (glioblastomas), mesothelioma, Triple-negative breast cancer, small cell lung cancer, and melanomas.

### X. ARTICLES OF MANUFACTURE OR KITS

An article of manufacture or a kit is provided comprising one or more inhibitors of CSF-1, one or more inhibitors of CSF-1R, one or more reagents and/or devices to measure for MRD, including for ctDNA, or a combination thereof. The article of manufacture or kit can further comprise a package insert comprising instructions for using the kit components to assay for MRD and/or to treat or delay progression of cancer (including MRD) in an individual. Any of the components encompassed in this disclosure may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds a formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

Any kit component may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits also will typically include a means for containing the kit component(s) in close confinement for commercial sale. Such containers may include injection or blow molded plastic containers into which the desired vials are retained. The kit can also include instructions for use, such as in printed or electronic format, such as digital format.

Individual components may also be provided in a kit in concentrated amounts; in some embodiments, a component may be provided individually in the same concentration as it would be in a solution with other components. Concentrations of components may be provided as 1x, 2x, 5x, 10x, or 20x or more.

### XI. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Anti-CSF1R Antibody Monotherapy Inhibits Minimal Residual Disease in a novel immunocompetent murine colorectal cancer metastasis model

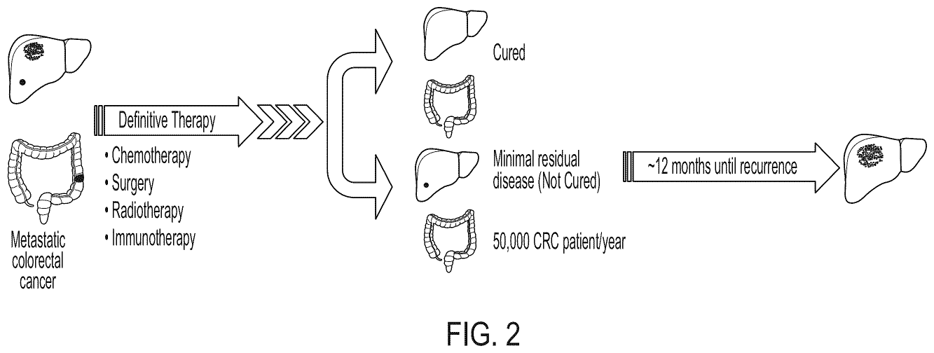

The high mortality rate of colorectal cancer (CRC) is primarily due to metastatic disease (FIG. 2). Microscopic minimal residual disease (MRD) detected in patients by circulating tumor DNA (ctDNA) may remain radiographically undetectable and persist following therapeutic intervention, resulting in recurrence. Preclinical models are urgently needed to elucidate the mechanisms whereby MRD escapes anti-tumor immune surveillance. The inventors have developed and characterized a preclinical liver MRD model that recapitulates immune response, with the goal to discover novel strategies to improve outcomes for patients with MRD, including in some cases with detectable ctDNA (ctDNA+ MRD).

The disclosure encompasses generation of genetically engineered mouse colonic organoids CDX2 CRE; Rosa26 LSL-CAS9-GFP with APC and TP53 mutation and performed intrasplenic injection in syngeneic C57BL/6 mice to recapitulate microsatellite stable CRC metastasis to the liver. Mice were monitored for radiographically detectable disease using MRI. Tumors were resected at various timepoints, with comparison between metastases at various sizes of development (dichotomized as micro-and macro-metastases). Multiplex immunofluorescence staining, single cell RNA seq, and spatial transcriptomic analysis were performed to delineate the immune compartments of micro-and macro-metastasis in untreated mice. After identifying colony stimulating factor-1 receptor (CSF1R) as a target, a vehicle controlled anti-CSF1R monotherapy study was performed for 4 weeks. Treatment began at either 7 days before metastasis is radiographically detectable to mimic treatment of MRD, or 21 days post tumor engraftment.

Immune exclusion was confirmed through intratumoral CD45+ leukocyte densities, with transition to an immune excluded phenotype occurring above 0.5 mm in size (defined as macro-metastases). In a preclinical model, immunosuppressive populations of macrophages (IBA1+CD163+) dominated in the micro-metastases. This observation was confirmed in 13 patients with paired micro-and macro-metastases to the liver (p=0.0065). Strikingly, anti-CSF1R treatment in the murine model eradicated IBA1+CD163+ macrophages (p<0.0001) and induced complete remission of liver micro-metastasis (0/7 mice with residual disease), in contrast to limited efficacy for macrometastases (5/5 mice with progressed radiographically disease). Additionally, a long-term study confirmed 100% survival (5/5 mice) and no evidence of disease via MRI at 6 months upon anti-CSF1R treatment compared to vehicle (0/5 mice).

A reproducible preclinical model of MRD with similar immunopathological features of MRD in patients. The data further identified infiltration of immunosuppressive macrophage populations as an early determinant of MRD progression and indicated CSF1R as a potential new clinical therapeutic target for ctDNA+-defined MRD patients.

### Example 2

### Immune Exclusion Associated with Early Liver CRC Metastatic Lesions

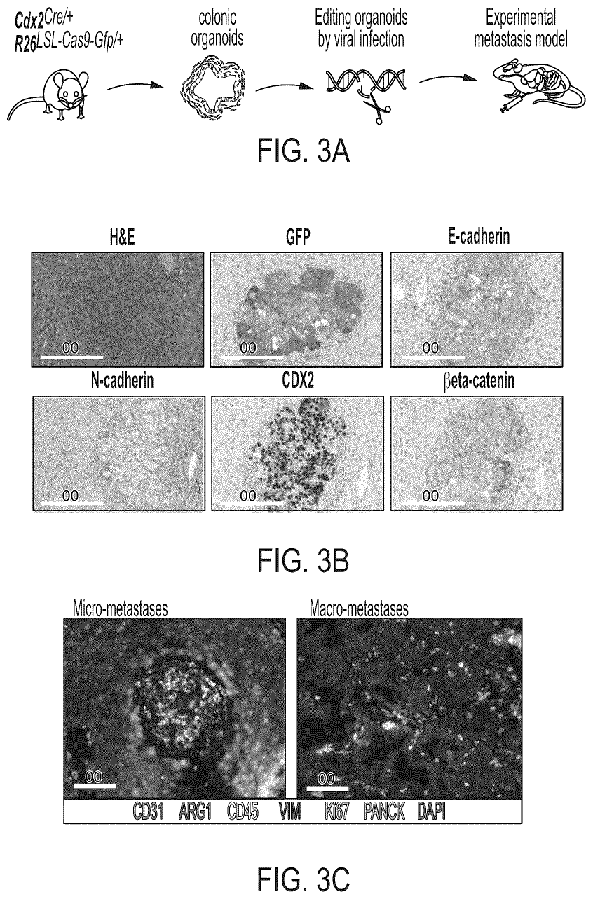

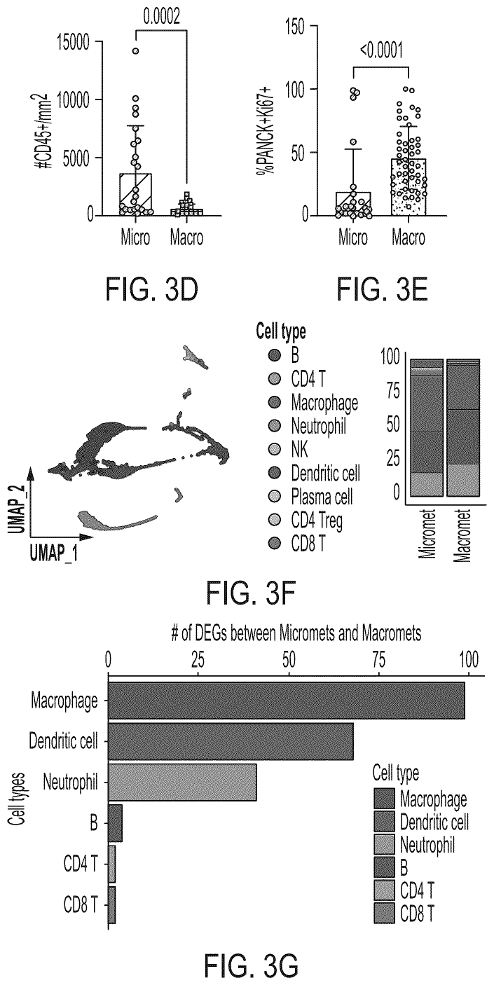

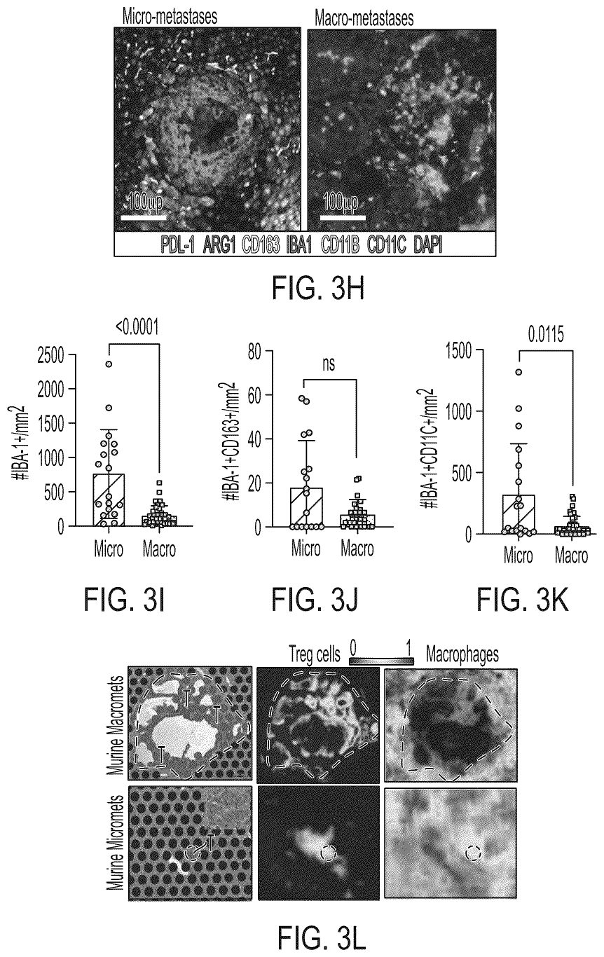

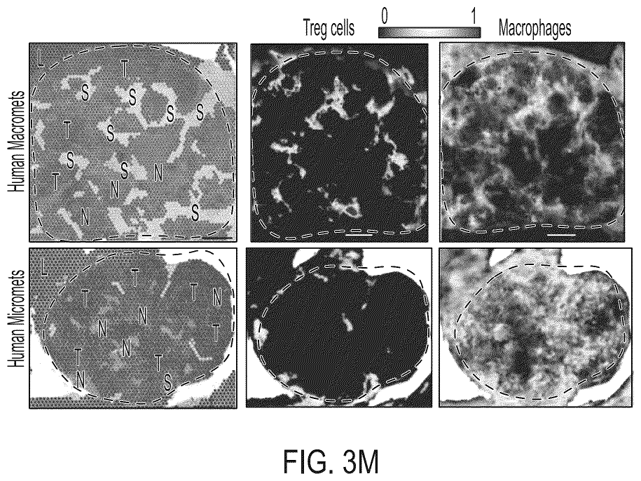

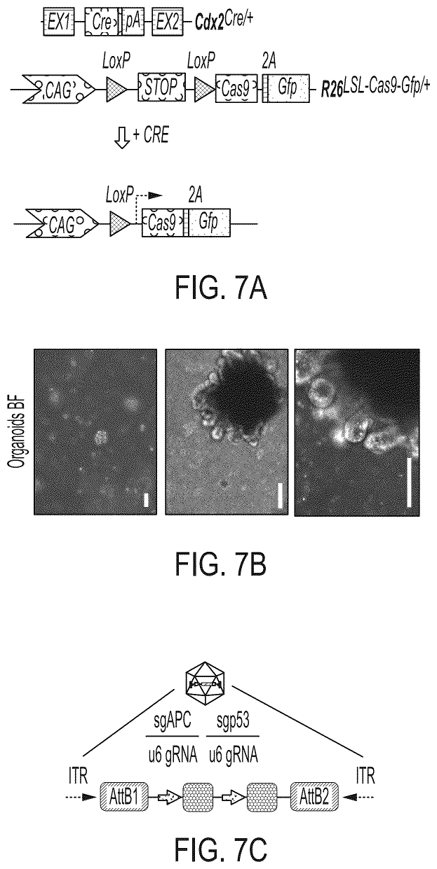

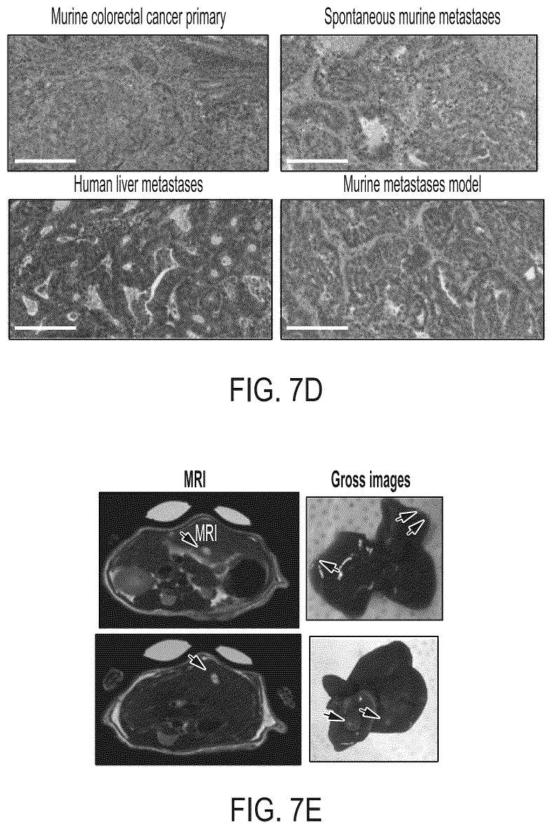

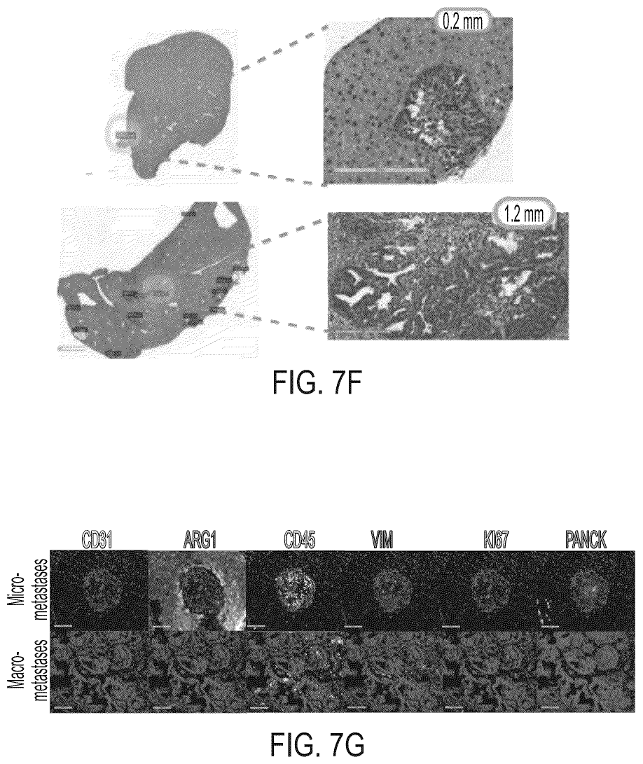

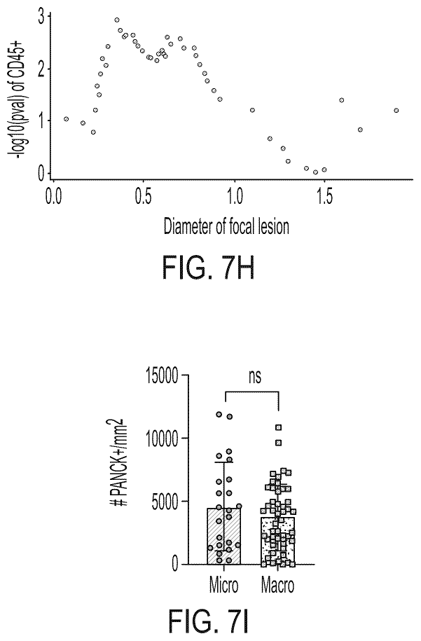

To generate genetically engineered murine colonic organoids recapitulating human mCRC molecular subtypes, Cdx2-CRE mice expressing Cre recombinase under the colon-specific Cdx2 promoter was crossed with R261.SL-CAS9-GFP mice, creating CDX2 CRE; Rosa26 LSL-CAS9-GFP mice, where Cas9 and the GFP expression were restricted to CDX2-positive cells (FIGS. 3A, 7A, and 7B). CRISPR-Cas9 editing was then used to introduce APC and TP53 mutations with adeno-associated virus-9 (AAV-9) particles harboring single guided RNAs (sgRNAs) of APC and TP53 to induce somatic gene editing (FIG. 7C). Organoids were injected subcutaneously into immunodeficient C57BL6/SCID mice, and tumors were transplanted into C57BL/6J mice over three rounds.

After submucosal colonic injection, primary colorectal cancer emerged after 6-8 weeks with liver metastases detected after 2 months (FIG. 7D). Using a modified intrasplenic injection technique, an experimental metastatic model was further established for studying early liver metastatic lesions with similarities by H&E with human liver metastasis (FIG. 7D). Immunohistochemistry (IHC) staining confirmed the epithelial origin of the experimental liver metastases (GFP+, E-CAD+, N-CAD-, CDX2+, and β-catenin⁺) and high CDX2 expression (FIG. 3B).

To assess early microscopic disease, intrasplenic injections were performed of 200K tumor cells and used magnetic resonance imaging (MRI) 12 days post-injection to track metastases development. Necropsies at day 13 revealed macroscopic liver metastases confirmed by H&E staining, with microscopic metastases from <0.1 mm to >2 mm detected by liver sections staining (FIGS. 7E and 7F).

The microenvironment of early metastatic lesions was next characterized using a multiplex immunofluorescence (mIF) panel on 71 metastases (FIGS. 3C and 7G). Because prior data has documented the exclusion of immune cells with enlarging metastases, CD45+ expression was evaluated as a function of tumor size and it was found that a diameter of 0.5 mm maximized the difference in CD45+ intratumoral density (p=0.0002) (FIG. 3D). Therefore, a size cutoff of 0.5 mm was established to distinguish between micro-metastases (diameter ≤0.5 mm) and macro-metastases (diameter>0.5 mm) (FIG. 7H). Findings from the mIF analysis revealed that proliferating tumor cells (PANCK+ KI67+) were more frequently detected in macro-metastases without any observable difference in density of tumor cells (PANCK+) in analyzed micro-and macro-metastases (FIGS. 3E and 7I). These findings confirm CD45+ exclusion as liver metastases grow, with micro-metastases remaining less proliferative.

### Example 3

### Immune Landscape of an Experimental CRC Liver Metastatic Model

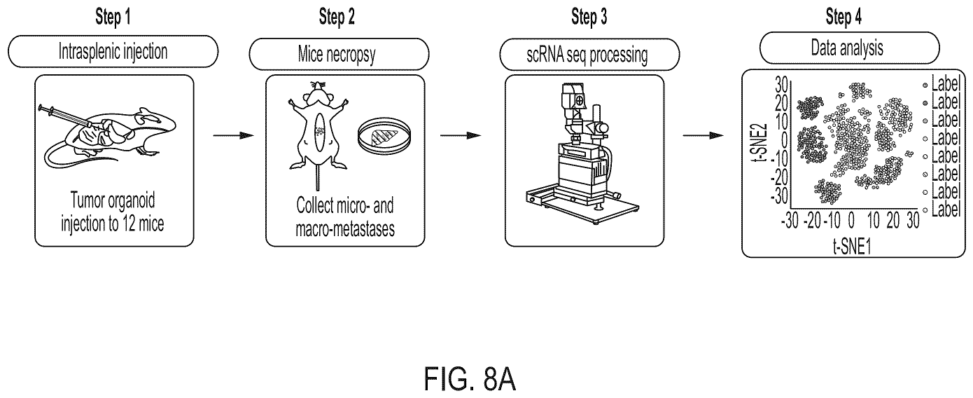

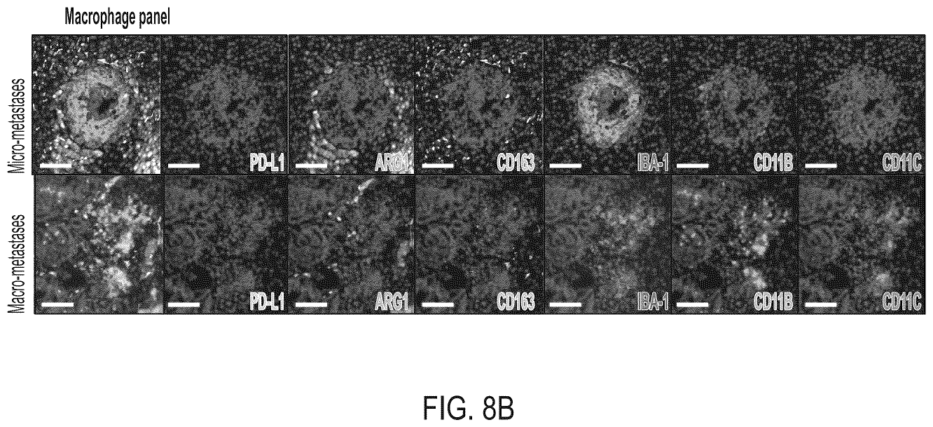

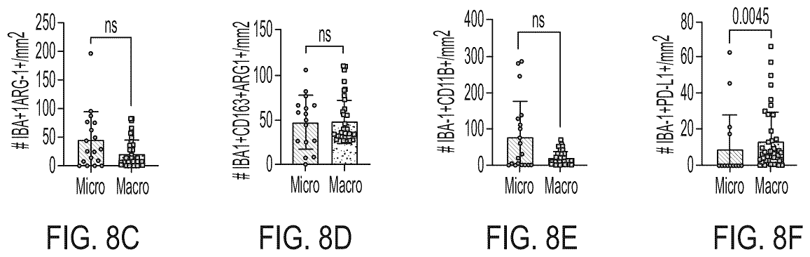

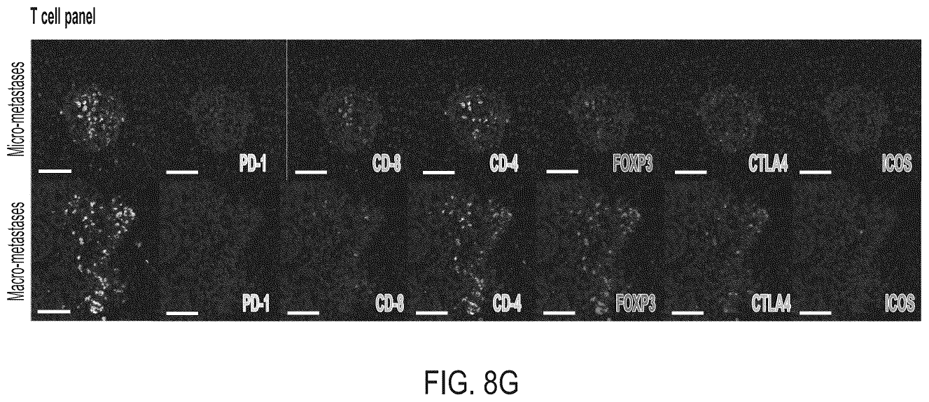

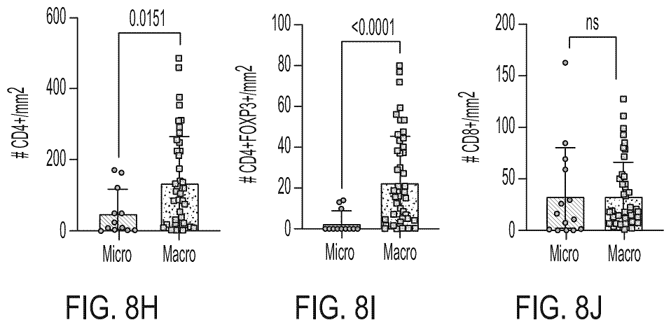

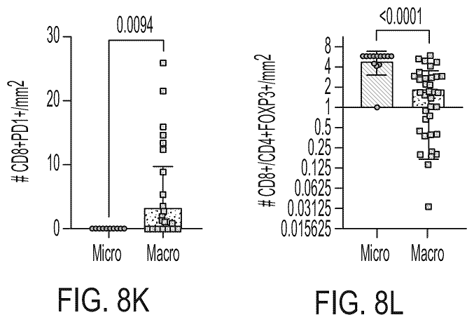

To delineate immune cell states and composition of micro-and macro-metastases, we performed single-cell RNA sequencing (scRNA-seq) on pooled metastases from 12 mice (FIG. 8A). Unsupervised clustering analysis revealed nine major immune cell types: B cells, CD4+ conventional T cells, CD4+ regulatory T cells, CD8+ T cells, macrophages, neutrophils, dendritic cells (DCs), plasma cells, and natural killer (NK) cells (FIG. 3F). Of note, macrophages and neutrophils were a greater proportion of the cellular population in macro-metastases, while fewer T-regs were observed in micro-metastases (FIG. 3F). Interestingly, compared to other immune cell types, the scRNA-seq data revealed a wide difference in differentially expressed genes (DEG) associated with macrophages between micro-and macro-metastases (FIG. 3G), suggesting an evolving role of macrophages in tumor progression.

To better quantitatively identify the immune tumor microenvironment, multiplex IF was performed that revealed higher IBA-1+ macrophages density in micro-metastases with pro-tumorigenic macrophages CD163+ and ARG1+ are infiltrating early the micro-metastases. There were similar levels of bone marrow-derived CD11B+ macrophages in both micro-and macro-metastases, whereas density of IBA-1+CD11C+ DCs and PDL-1+ macrophages were enriched in macro-metastases (FIGS. 3H-3K and FIGS. 8B-8F). These findings suggest that pro-tumorigenic CD163+ and ARG1+ macrophages are activated early to support an immune suppressive microenvironment, facilitating early liver metastases initiation. Profiling of T-cell populations demonstrated higher density of CD4+ conventional T cells and CD4+FOXP3+ Tregs in macro-metastases. Conversely, the density of total CD8+T cells were similar in both micro-and macro-metastases, whereas PD1+CD8+ T cells were significantly increased in macro-metastases, with a reduced CD8+/Treg ratio (FIGS. 8G-8I), indicating T cell-mediated immunosuppression increases as micro-metastases progress into macro-metastases.

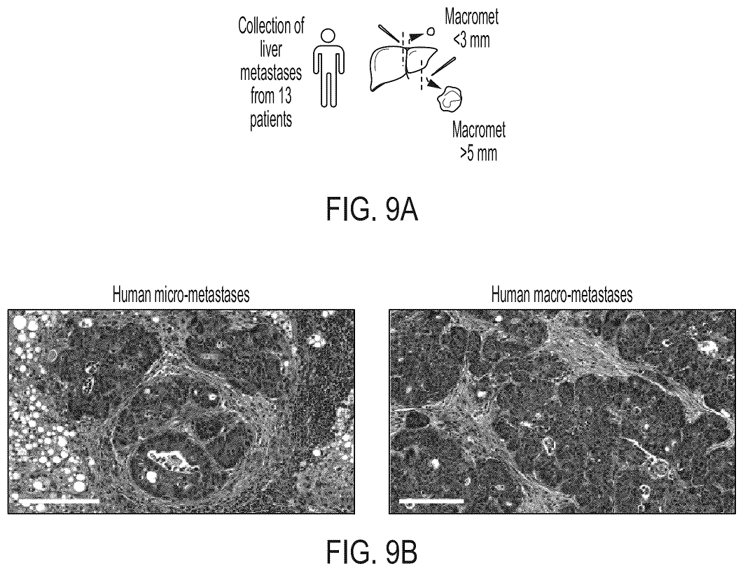

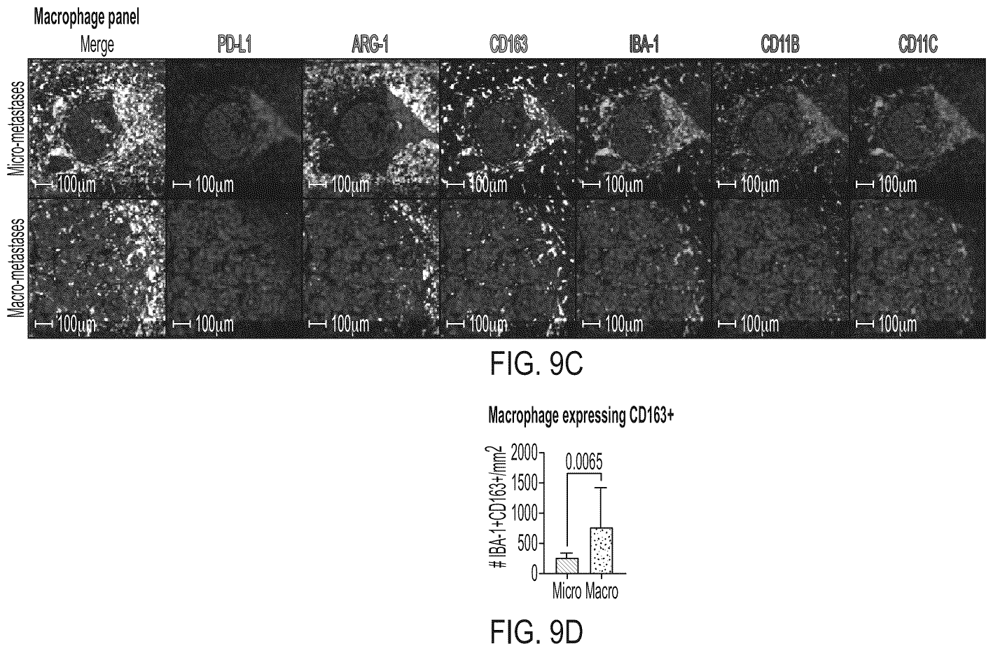

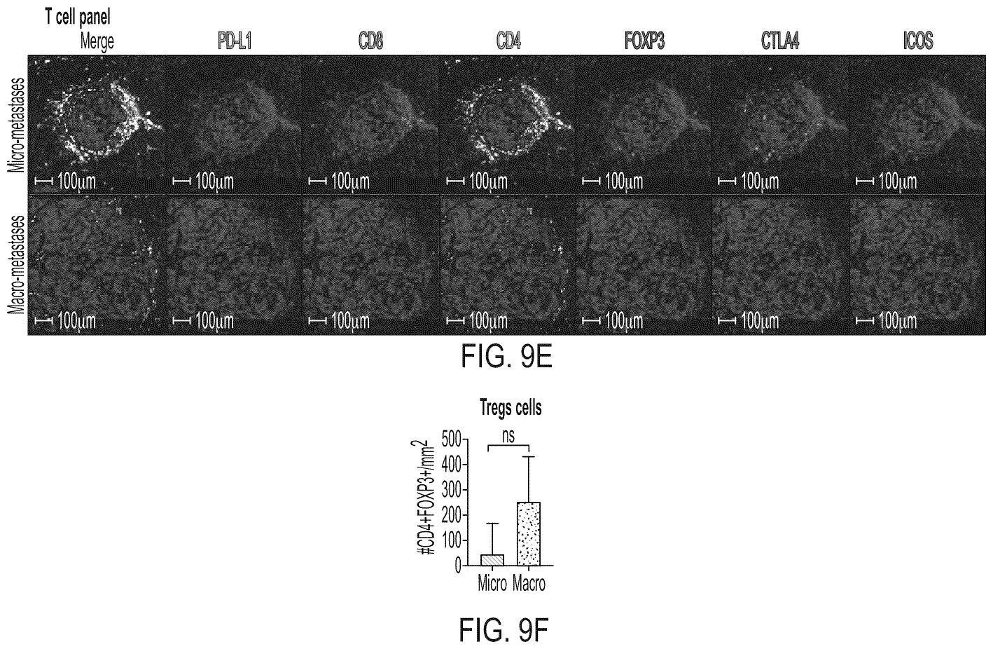

To validate the findings on clinical samples, multiplex IF panels were conducted on 22 patient liver metastases that were classified based on the clinical definition of MRD, wherein lesions smaller than 3 mm in diameter and not radiographically detectable were categorized as micro-metastases, and lesions larger than 3 mm in diameter were categorized as macro-metastases (FIGS. 9A and 9B). Human CRC liver metastases showed high infiltration of anti-inflammatory CD163+ macrophages early in micro-metastases with significant increase in macro-metastases (p=0.0065), as well as low infiltration of Tregs in micro-metastases while the density of Tregs increases as tumor grows. Patients' quantification results validate the murine findings for the importance of macrophages in micro-metastases (FIGS. 9C-9E).

It was next sought to confirm the enrichment of macrophages in murine and human CRC liver metastases samples using spatial transcriptomic (ST, 10X Genomics). To achieve this, a iStar18 machine was employed, which is a machine learning tool that integrates ST data and histology images, enabling resolution enhancement and cell type annotations through curative gene signatures to spatially map macrophages and Tregs in murine and human samples. The results showed Treg were enriched in macro-metastases compared to micro-metastases, whereas macrophages were enriched in micro-metastases compared to macro-metastases (FIGS. 3L and 3M). Overall, the data underscore the significance of macrophages, particularly anti-inflammatory CD163+ macrophages, in early microscopic metastatic lesions.

### Example 4A

### CSF1R High Macrophage Cluster Predominance in Liver Micro-Metastases

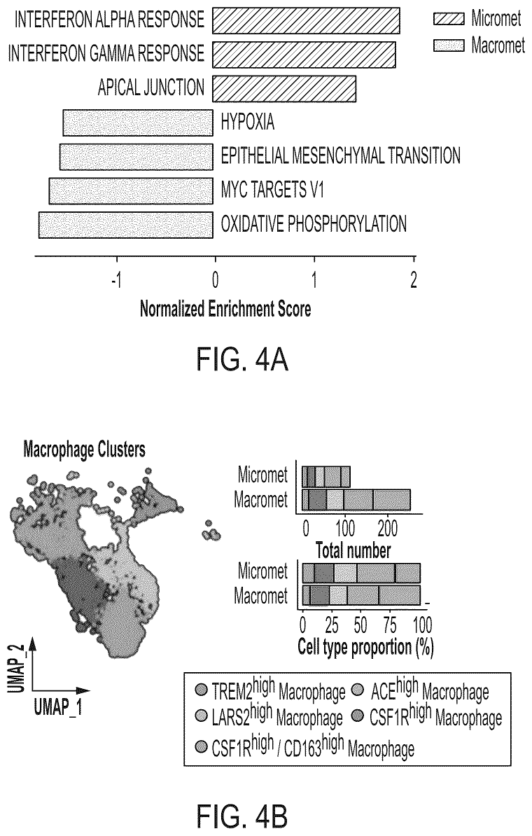

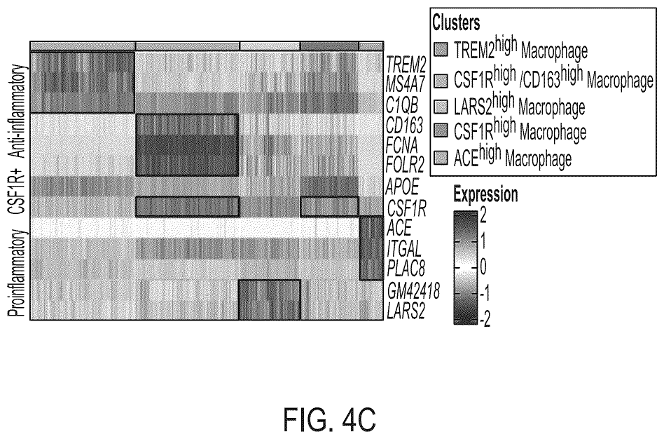

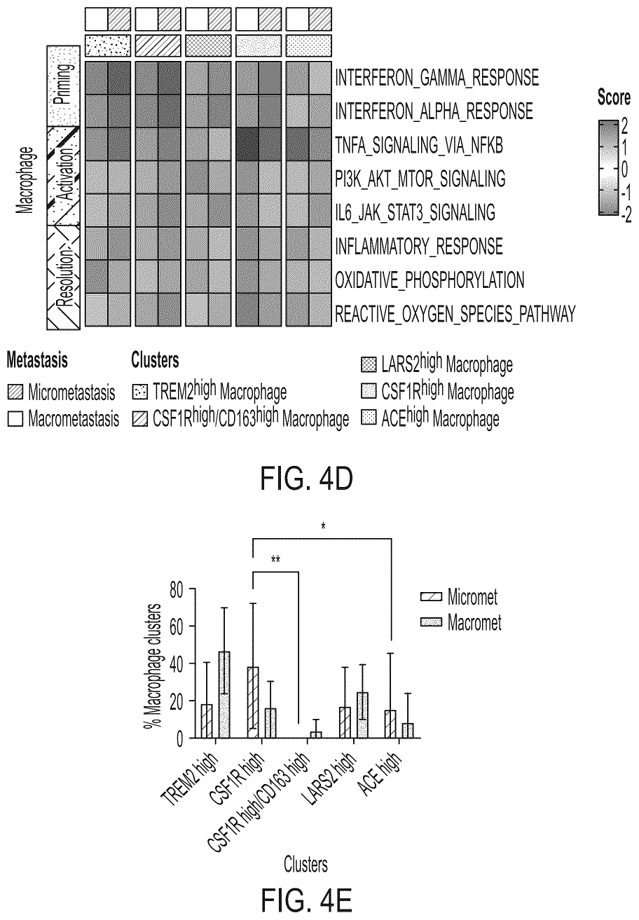

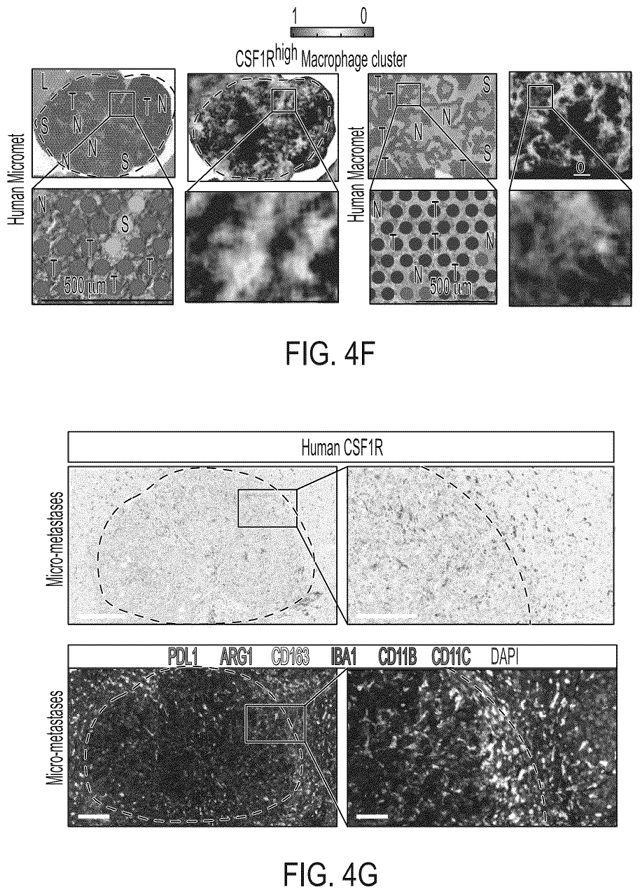

To explore transcriptomic differences in macrophage population between micro- and macro-metastases, hallmark pathways were examined, finding significant upregulation of interferon alpha/gamma responses and apical junction pathways in micro-metastases, while hypoxia, epithelial mesenchymal transition, MYC targets, and oxidative phosphorylation pathways were notably upregulated in macro-metastases (FIG. 4A). scRNA-seq revealed five distinct macrophage clusters in both micro-and macro-metastases, with the TREM2 and the CSF1R/CD163 high clusters exhibiting anti-inflammatory gene signatures, whereas the LARS2 and ACE high cluster displaying pro-inflammatory gene signatures (FIGS. 4B and 4C). Hallmark pathway analysis showed upregulated macrophage priming pathways in micro-metastases all clusters, with the CSF1R high cluster showing downregulated macrophage activation and resolution pathways in micro-metastases, indicating a transitional phenotype for this cluster between pro-and anti-inflammatory states (FIG. 4D). To assess potential differences in gene enrichment among macrophage clusters in micro-metastases, CytoSPACE analysis was conducted, aligning scRNA-seq annotations for the macrophage clusters with the spatial data, in murine micro-and macro-metastases. Results revealed the enrichment of the CSF1R high cluster in micro-metastases compared to other macrophage clusters and relative to macro-metastases (FIG. 4E). Follow-up iStar analysis confirmed the enrichment of the CSF1R high cluster in micro-compared to macro-metastases from patient samples (FIG. 4F), thus indicating the significance of the CSF1R high macrophage cluster in both murine and human micro-metastases. These data were further validated using IHC and multiplex IF analysis on human liver metastases, which confirmed high infiltration of macrophages expressing IBA-1+ and CSF1R+ expression in micro-metastases (FIG. 4G).

Together, the results highlight the scarcity of Tregs and enrichment of macrophages in micro-metastases, particularly the CSF1R high cluster with a plastic phenotype, indicating CSF1R is a useful therapeutic target in micro-metastases.

### Example 4B

### Eradication of Liver Micro-Metastases with Anti-CSF1R Monotherapy

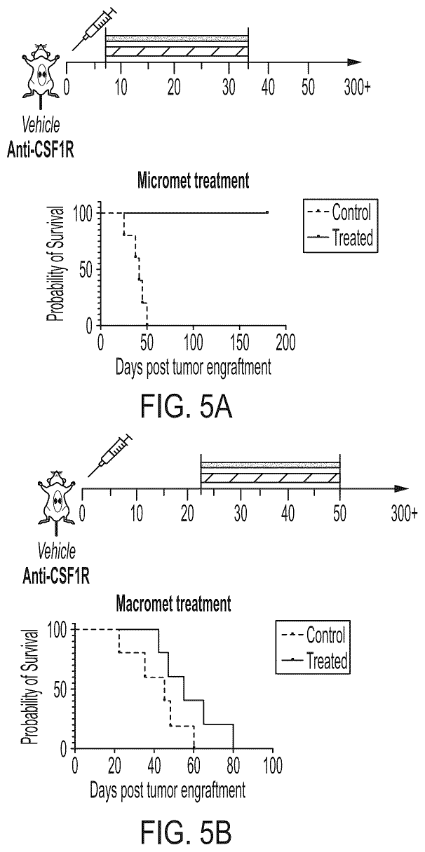

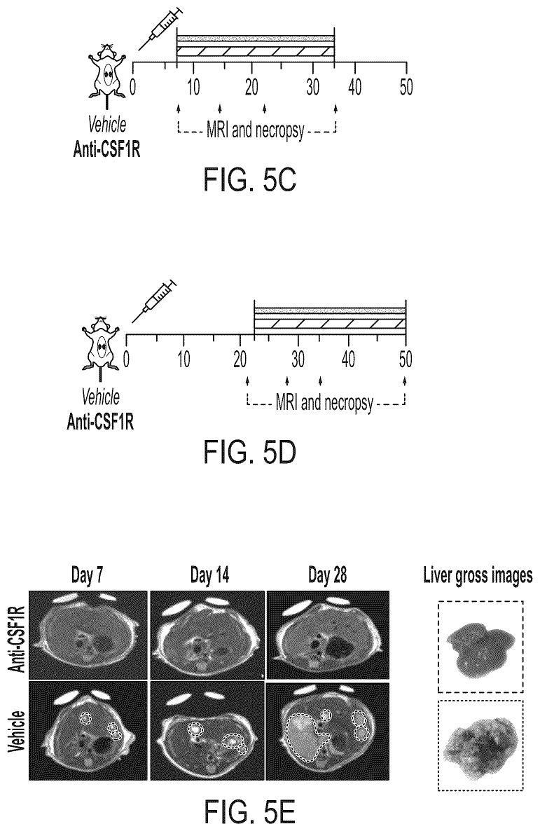

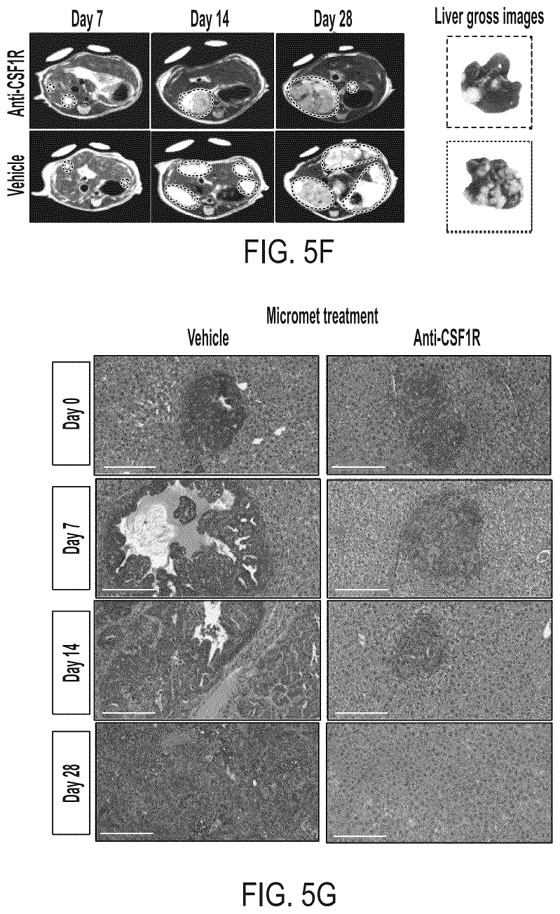

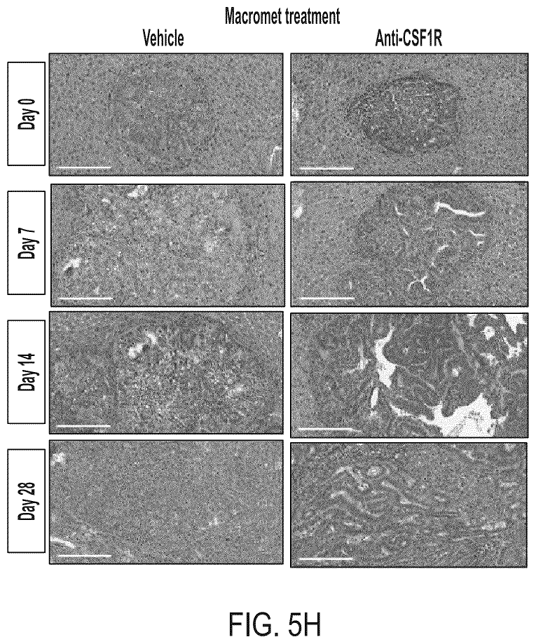

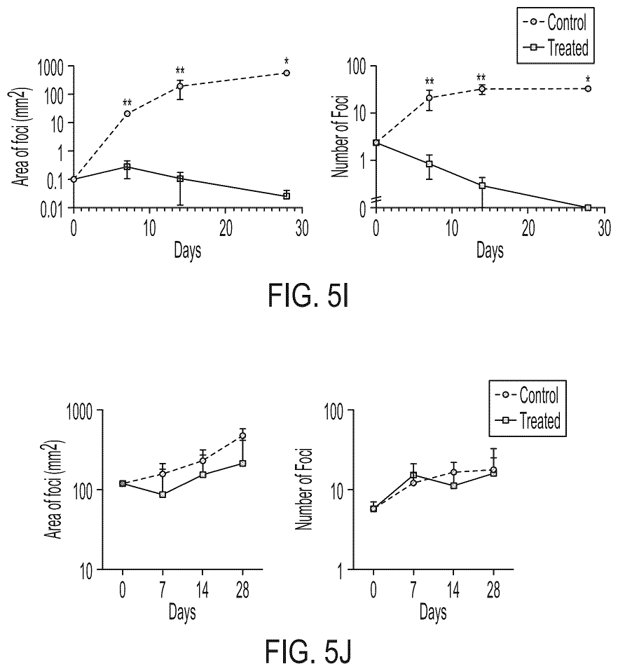

The inventors evaluated the survival impact of anti-CSF1R treatment in models with microscopic and macroscopic disease by administering anti-CSF1R antibody or rat Ig2a isotype (control) at 7 (micro-metastases) or 21 days (macro-metastases) post-intrasplenic injection. Kaplan-Meier survival analysis revealed a significant survival benefit in the micro-metastases group treated with anti-CSF1R, but not in those with macro-metastases (FIGS. 5A and 5B).

To assess whether targeting CSF1R-high macrophages halted the progression of micro-or macro-metastases, the inventors repeated the aforementioned treatment regimen and monitored tumor growth weekly using MRI imaging, and mice was sacrificed after MRI imaging for histological examination (FIGS. 5C and 5D). MRI monitoring and gross necropsy revealed that anti-CSF1R halted tumor progression in mice with micro-metastases, but not in those with macro-metastases, where tumor progression occurred in both treated and control groups (FIGS. 5E and 5F). Histological analysis confirmed only two metastases out of 7 mice after 14 days of anti-CSF1R treatment and complete eradication of micro-metastases upon 4 weeks of anti-CSF1R treatment, whereas macro-metastases continued to progress regardless of treatment (FIGS. 5G and 5H). Quantitative analysis further verified the efficacy of anti-CSF1R in eliminating micro-metastases, but not macro-metastases (FIGS. 5I and 5J).

### Example 5

### Anti-CSF1R Therapy Altered the Immune Landscape in Liver Micro-Metastases

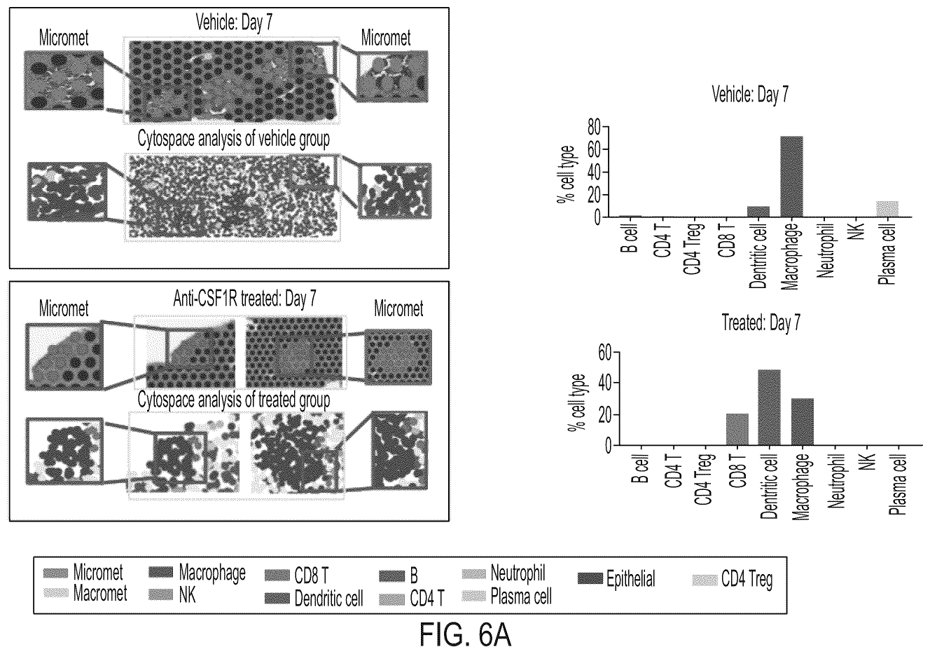

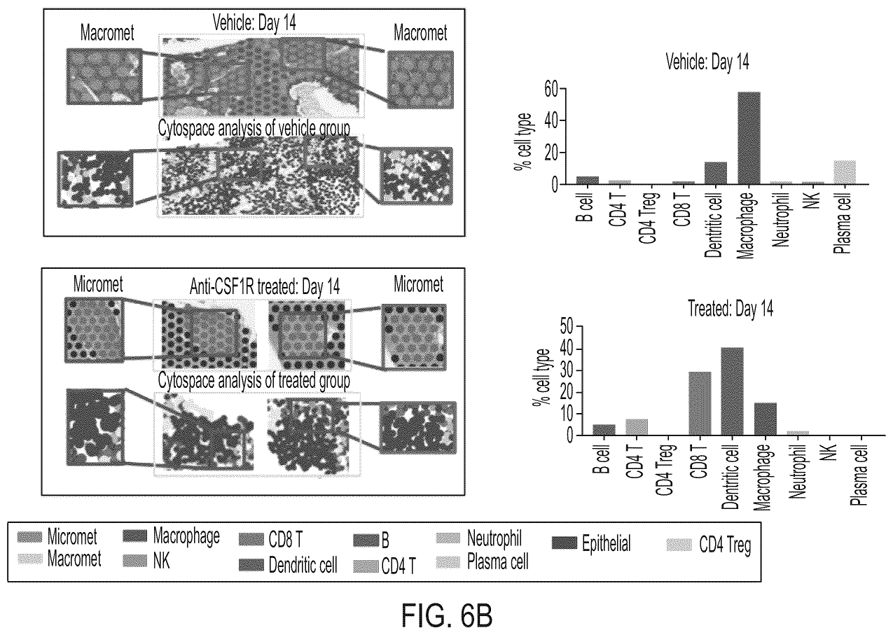

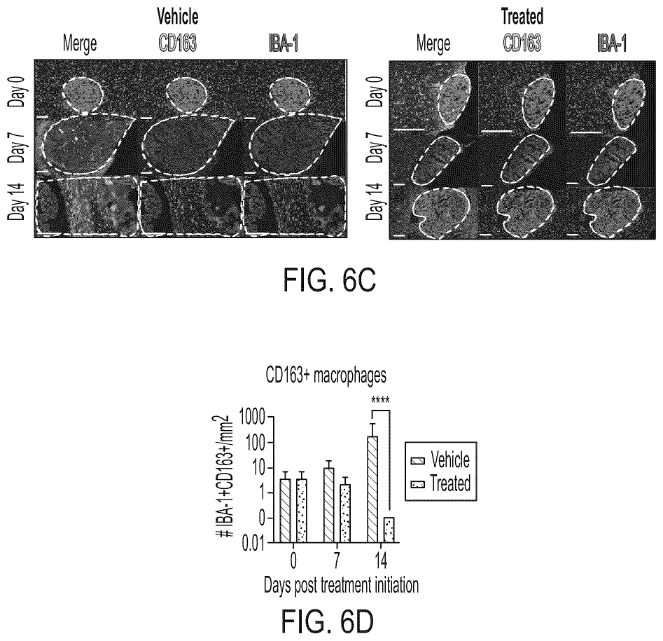

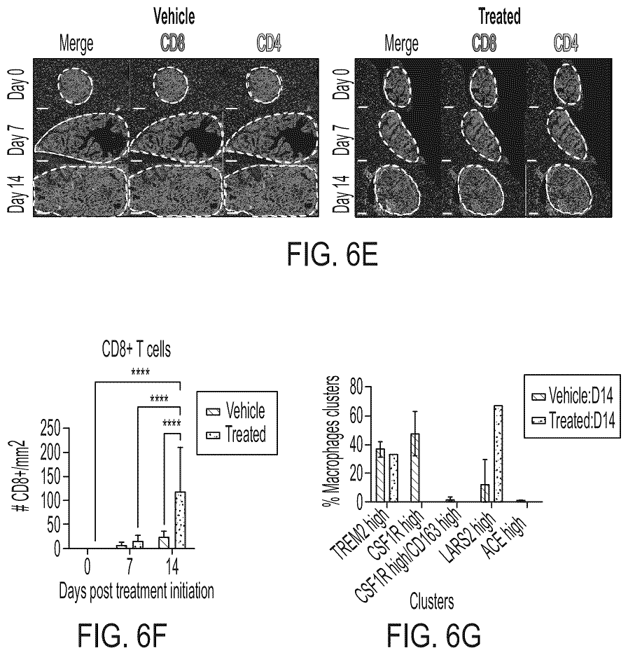

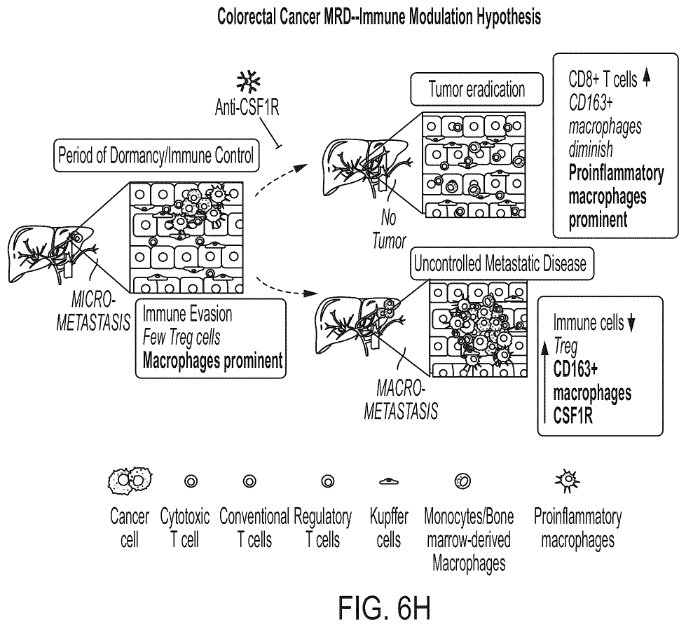

To investigate the differential immune effect of anti-CSF1R treatment on liver micro-metastases, spatial transcriptomics (CytoSPACE) analysis was performed of immune cell types on liver metastases collected on Day 7 and 14. From control group at 7 days to anti-CSF1R treatment for 14 days, macrophages were depleted from 71% to 15%, while CD8+ T cells increased from 0.1% to 30% (FIGS. 6A and 6B).

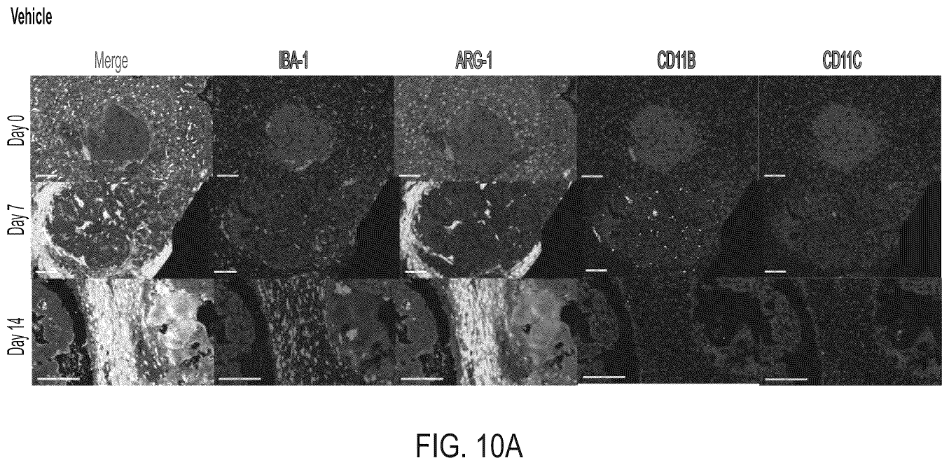

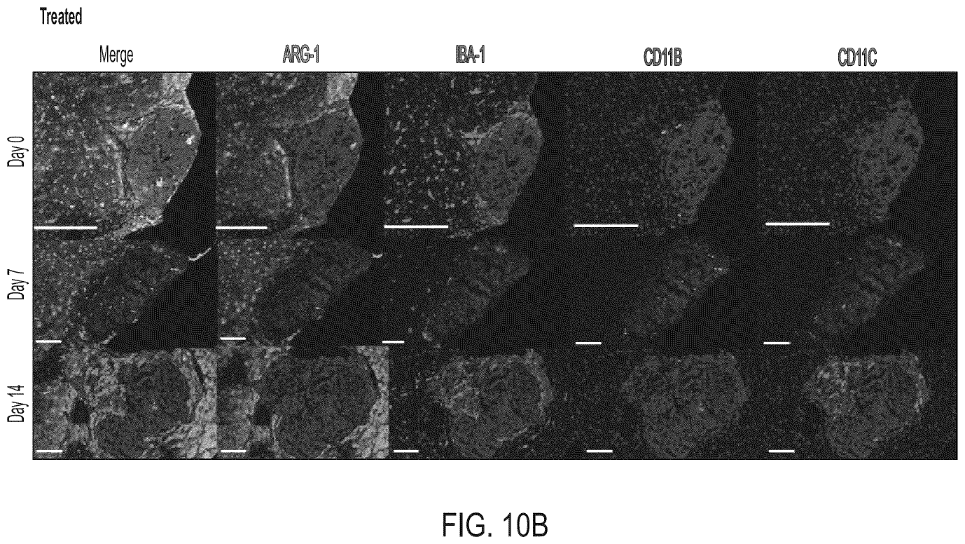

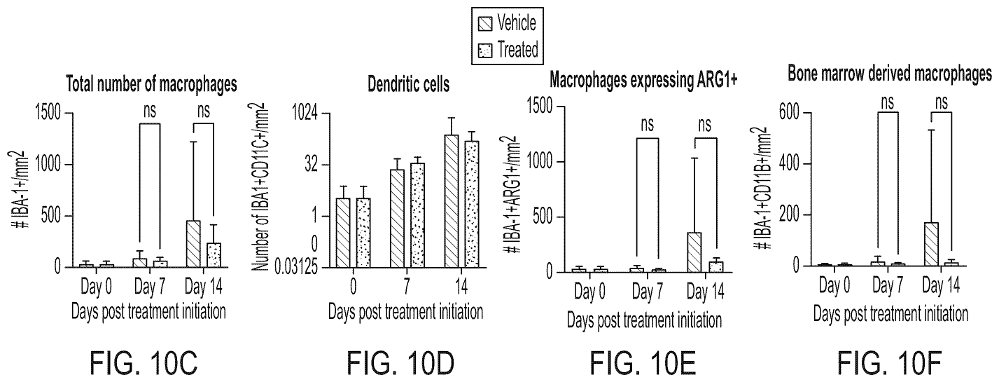

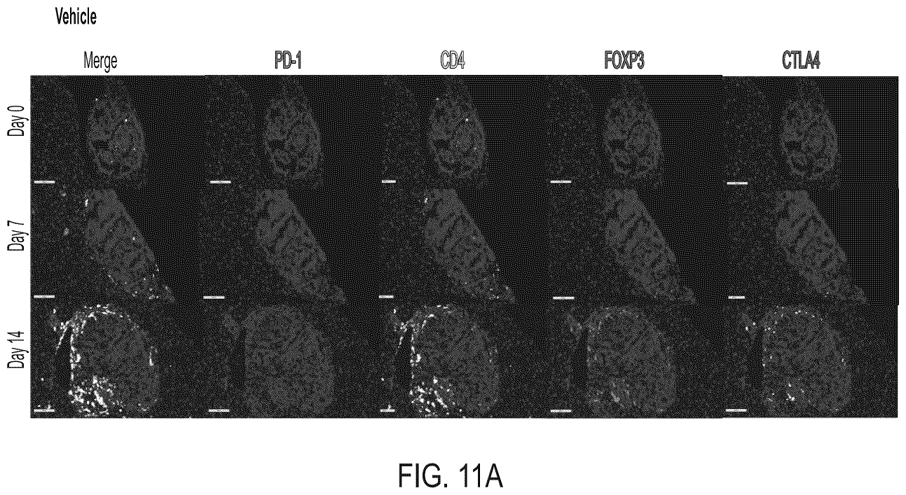

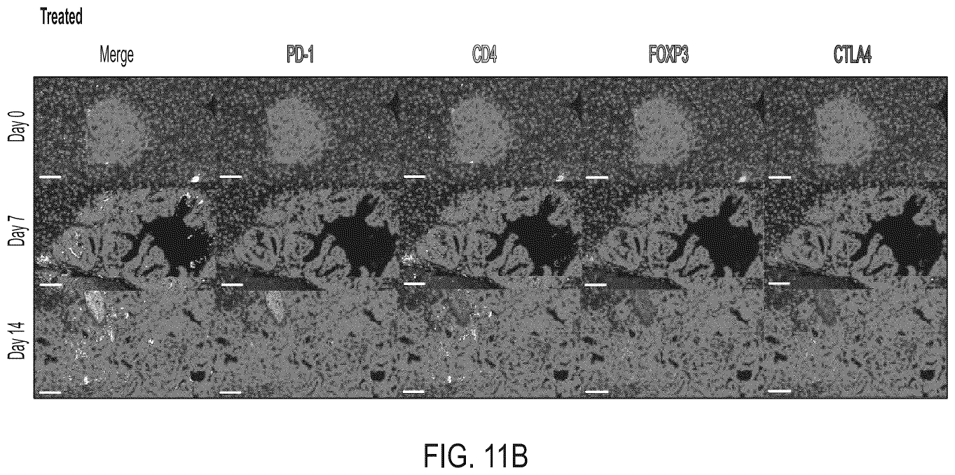

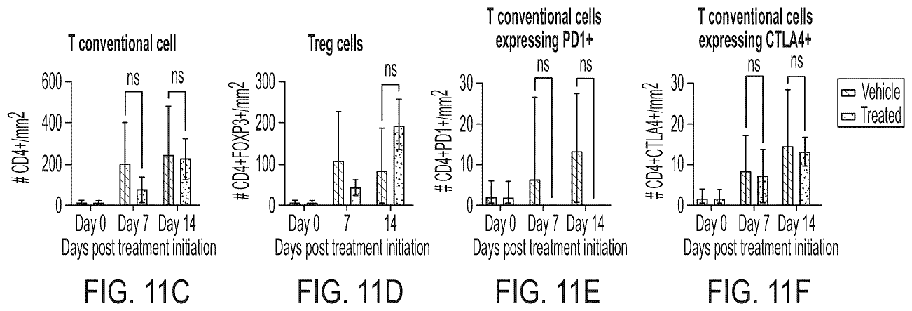

Macrophage and T cell multiplex IF panels validated these findings, showing complete depletion of IBA-1+CD163+ macrophages by day 14 (p<0.0001) (FIGS. 6C and 6D), with reduction in density of macrophages (IBA-1+), bone marrow derived macrophages (IBA-1+CD11B+), and macrophages expressing ARGI+ (IBA1+ARG1+) as well as no observable difference in dendritic cells (IBA1+CD11C+) (FIGS. 10A-10F), and increased CD8+T cells (p=0.0195) (FIGS. 6E and 6F), with reduction in T conventional cells expressing PD1+ (CD4+PD1+) as well as no significant difference in the total number of CD4+ conventional T cells, Treg, nor CTLA4+ CD4+ conventional T cells (FIGS. 11A-11F). These data suggest that anti-CSF1R treatment primarily acts on depleting macrophages in micro-metastases, particularly those expressing CD163+, as well as enhancing CD8+ T cell levels.

Further, CytoSPACE analysis revealed significant enrichment in the LARS2 high macrophage cluster (67%) and, to a lesser degree, in the TREM2 high cluster (33%) following 14 days of micro-metastases anti-CSF1R treatment (FIG. 6G), indicating a shift from anti-inflammatory macrophage clusters (TREM2 and CSF1R/CD163) and CSF1R high cluster to the pro-inflammatory LARS2 high cluster following anti-CSF1R treatment.

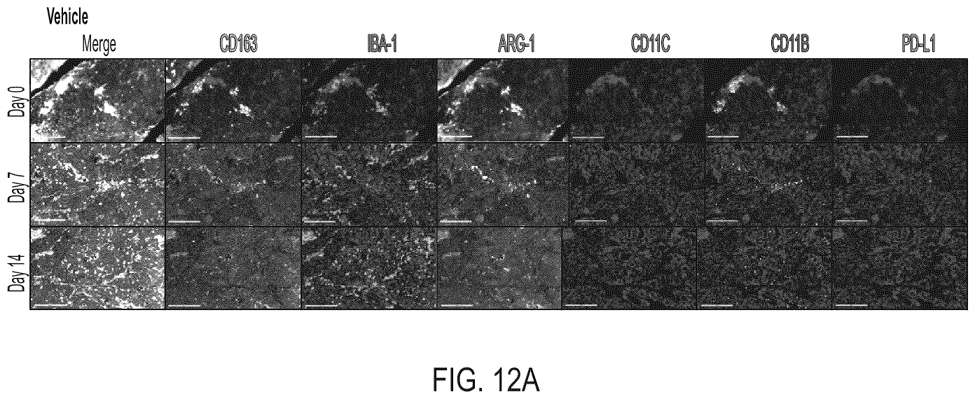

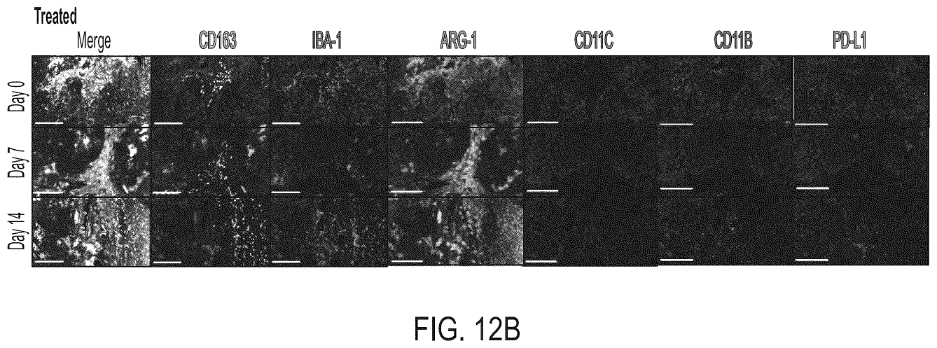

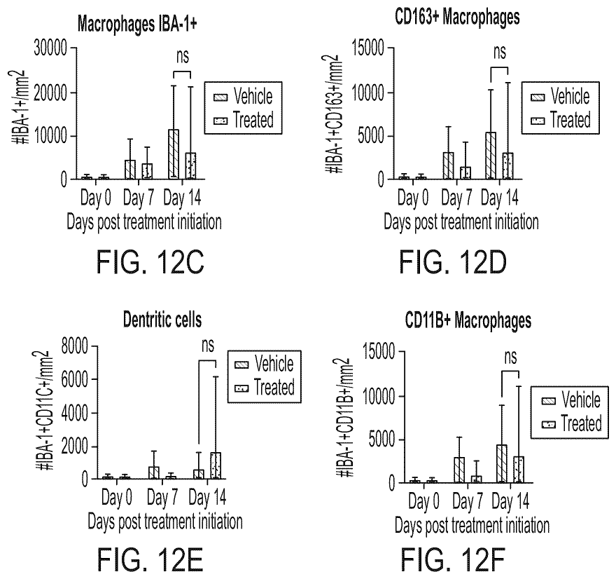

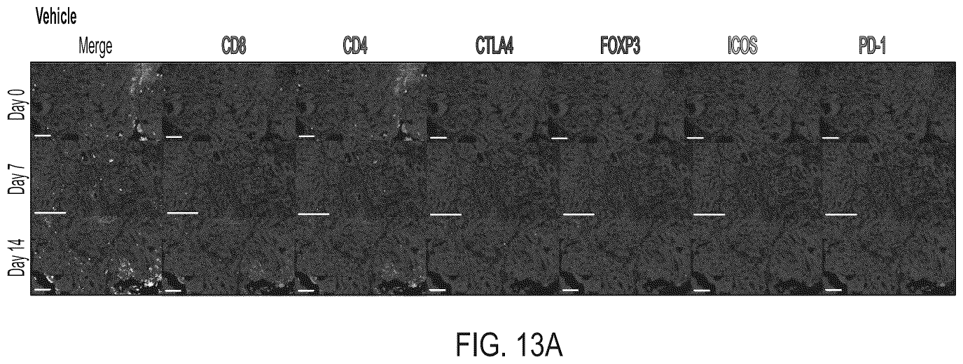

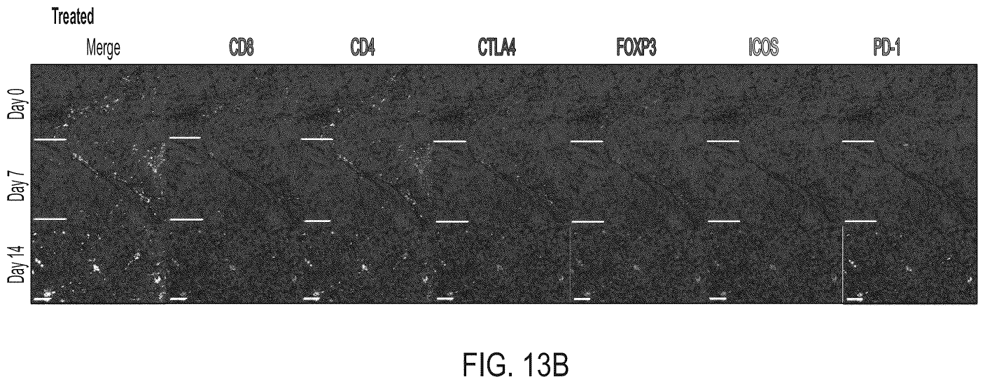

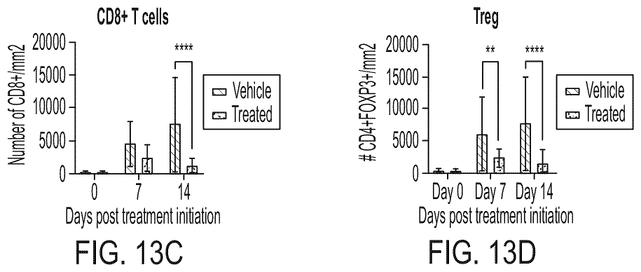

Conversely, in macro-metastases anti-CSF1R treatment, macrophage and T cell multiplex IF panels revealed failure of anti-CSF1R to deplete total IBA-1+, CD163+, and CD11B+ macrophages (FIG. 12) as well as to increase CD8+T cells or to deplete Tregs (FIG. 13), highlighting anti-CSF1R treatment inefficacy in macro-metastases context.

Overall, the results demonstrate that anti-CSF1R treatment eliminates micro-metastases by depleting tissue resident macrophages CD163+and CSF1R high macrophage cluster, promoting proinflammatory LARS2 high macrophage cluster, and enhancing CD8+T cell infiltration. However, this treatment is ineffective in macro-metastases, where both macrophages and CD8+T cells remain unchanged or are reduced (FIG. 6H).

### Example 6

### Measuring MRD in a Genetically Engineered Murine Model for Liver MRD

Radiologically, MRI can barely detect microscopic lesions above 0.5 mm in diameter. In particular aspects, MRD size was characterized in the liver preclinical model encompassed herein. In specific aspects, a pre-clinical immune conversion cut-point size correlating to a liver MRD size is defined based on statistical analysis of CD45+ expression in macrometastases and micromestastases. In certain aspects, the size cut point to distinguish micro-metastasis (<0.5 mm) compared to macro-metastasis is on the verge of >0.5 mm.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

1. A method of treating minimal residual disease in an individual, comprising administering to the individual a therapeutically effective amount of an agent that is one or more inhibitors of colony stimulating factor 1 (CSF-1) and/or one or more inhibitors of colony stimulating factor 1 receptor (CSF-1R).

2. The method of claim 1, wherein the individual has circulating tumor DNA (ctDNA).

3. The method of claim 2, wherein the individual has undetected or undetectable cancer cells.

4. The method of claim 3, wherein the individual has radiographically undetectable cancer cells.

5. The method of claim 1, wherein the individual has no detected ctDNA but is at risk of developing MRD or is suspected of having MRD.

6. The method of claim 1, wherein the minimal residual disease is characterized with high microsatellite instability (MSI-H), low microsatellite instability (MSI-L) or microsatellite stability (MSS).

7. (canceled)

8. The method of claim 1, wherein the individual has received therapy for a cancer.

9. (canceled)

10. (canceled)

11. The method of claim 8, wherein the the cancer is colorectal, skin, breast, lung, liver, stomach, brain, thyroid, blood, gall bladder, ovarian, cervical, testicular, prostate, kidney, pancreatic, small bowel, gastric, mesenteric, GI lymph node, colon, or bladder cancer, or any cancer that engages the portal vein and/or drains into the liver.

12. The method of claim 11, wherein the cancer is colorectal cancer that has metastasized to the liver.

13. (canceled)

14. (canceled)

15. (canceled)

16. (canceled)

17. (canceled)

18. The method of claim 1, wherein the agent is an antibody to CSF-1 or CSF-1R.

19. The method of claim 18, wherein the antibody is AMB001.

20. The method of claim 18, wherein the antibody is selected from the group consisting of emactuzumab, cabiralizumab, axatilimab, IMC-CS4, lacnotuzumab or PD-0360324.

21. The method of claim 1, wherein the agent is a small molecule.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. A method of treating an individual after one or more cancer therapies, comprising administering after the cancer therapy to the individual a therapeutically effective amount of one or more inhibitors of CSF-1 and/or one or more inhibitors of CSF-1R if the individual has ctDNA.

34-39. (canceled)

40. A method of preventing or delaying the onset of cancer recurrence in an individual, comprising administering to the individual after one or more cancer therapies a therapeutically effective amount of one or more inhibitors of CSF-1 and/or one or more inhibitors of CSF-1R if the individual has ctDNA.

41-46. (canceled)
